(19) United States
(12) Patent Application Publication
(10) Pub. No.: US 2026/0277103 A1
(43) Pub. Date: Sep. 17, 2026

(54) RADIATION-SENSITIVE COMPOSITION, RESIST PATTERN FORMATION METHOD, POLYMER, AND COMPOUND

(71) Applicant: JSR CORPORATION, Tokyo (JP)

(72) Inventors: Fumiya ESAKI, Tokyo (JP); Motohiro SHIRATANI, Tokyo (JP)

(73) Assignee: JSR CORPORATION, Tokyo (JP)

(21) Appl. No.: 19/164,836

(22) PCT Filed: Feb. 26, 2024
(86) PCT No.: PCT/JP2024/006854

§ 371 Date: Sep. 12, 2025

(30) Foreign Application Priority Data
Mar. 16, 2023 (JP) 2023-042261

(51) Int. Cl. G03F 7/039; C08F 212/14; C08F 220/18; C08F 220/22; C08F 220/30; C08F 220/38; C08F 220/40; G03F 7/004

(52) U.S. Cl. CPC G03F 7/039; C08F 212/24; C08F 220/1806; C08F 220/1809; C08F 220/1811; C08F 220/22; C08F 220/301; C08F 220/38; C08F 220/40; G03F 7/0045

(57) ABSTRACT

A polymer containing a structural unit represented by formula (1) is included in a radiation-sensitive composition. In formula (1), R¹ represents a hydrogen atom, a fluorine atom, a methyl group, or a trifluoromethyl group. X¹ represents a single bond, *¹—COO—, *¹—CONH—, or a divalent aromatic cyclic group. W¹ represents a (r+2)-valent organic group. X² represents *³—COO— or —O—. G¹ represents an acid-releasable group. r is 1 or 2. R² and R³ independently represents a fluorine atom or a fluoroalkyl group. M^{a+} represents an a-valent cation.

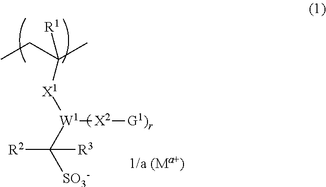

### TECHNICAL FIELD

### Cross-Reference to Related Applications

The present application claims a priority from Japanese Patent Application No. 2023-42261 filed on Mar. 16, 2023, the entirety of the disclosure of which is incorporated herein by reference.

The present disclosure relates to a radiation-sensitive composition, to a resist pattern formation method, to a polymer, and to a compound.

### BACKGROUND ART

In a lithography technique employed in production of various electronic devices including semiconductor devices and liquid crystal devices, a process target formed of a radiation-sensitive composition is irradiated with a far-UV ray (e.g., ArF excimer laser light), an extreme UV (EUV) ray, an electron beam, or the like, to thereby generate acid in a light-exposed part. Through chemical reaction involving the generated acid, difference in dissolution rate with respect to a developer is provided between the light-exposed part and the light-unexposed part. Thus, a resist pattern is formed on a substrate.

Meanwhile, structures of such electronic devices have been further miniaturized steeply. Under such circumstances, further fine resist patterns are required in lithography steps. In addition, in order to satisfy the requirement of further fine resist patterns, various studies have been done for improving resolution of a radiation-sensitive composition employed in lithographic micro-processing, shape characteristics of a resultant resist pattern, and the like (see, for example, Patent Document 1). Patent Document 1 discloses incorporation, into a radiation-sensitive composition, of a resin having a repeating unit which generates an acid via decomposition through irradiation with an active light ray or a radiation.

### PRIOR ART DOCUMENTS

### Patent Documents

Patent Document 1: Japanese Patent Application Laid-Open (kokai) No. 2011-154216

### SUMMARY OF THE INVENTION

### Problems to be Solved by the Invention

Meanwhile, in a rapid progress in further process shrinkage of resist patterns in recent years, attempts have been made to form a pattern having, for example, a line width of 40 nm or less. Thus, the radiation-sensitive composition must provide a suitable resist pattern by a small dose (i.e., with high sensitivity), when the composition is also employed in formation of such fine resist patterns. In addition, the resist pattern must have excellent lithographic characteristics such as line width roughness (LWR) performance.

When the radiation-sensitive composition is irradiated with light, the acid generated in the light-exposed part diffuses to the light-unexposed part in some cases. In such a case, resolution of the resist film decreases, thereby failing to provide a fine pattern. On the other hand, along with further down-scaling of resist patterns, only a slight variation in process conditions (e.g., conditions for exposure and development) readily causes morphological problems, occurrence of failures, and the like which are involved in resist patterns. Thus, the radiation-sensitive composition must have such wide allowance that can accept slight variation in process conditions. That is, there is demand for wide ranges of process conditions which ensure formation of a pattern having no breakage and collapse in the resist pattern formation step (hereinafter, such a range may also be referred to as a “process margin”).

The present disclosure has been made in view of the aforementioned problems. Thus, an object of the present disclosure is to provide a radiation-sensitive composition which can provide both high sensitivity and suitable LWR performance and a sufficiently wide process margin during formation of a pattern. Another object is to provide a resist pattern forming method.

### Means for Solving the Problems

According to one aspect of the present disclosure, there is provided a radiation-sensitive composition containing a polymer including a structural unit represented by the following formula (1):

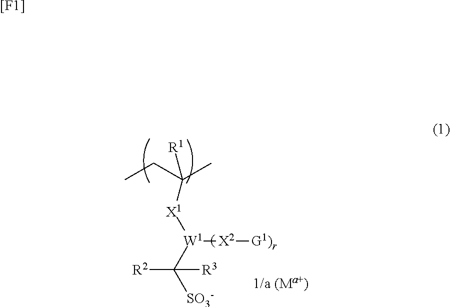

(wherein R¹ represents a hydrogen atom, a fluorine atom, a methyl group, or a trifluoromethyl group; X¹ represents a single bond, *¹—COO—, *¹—CONH—, or a divalent aromatic cyclic group; “*¹” represents a chemical bond with a carbon atom to which R¹ is bound; W¹ represents a (r+2)-valent organic group; X² represents *³—COO— or —O—; “*³” represents a chemical bond with W¹; G¹ represents an acid-releasable group; r is 1 or 2; when r is 2, two G¹s in the formula are identical to or different from each other; each of R² and R³ independently represents a fluorine atom or a fluoroalkyl group; M^{a+} represents an a-valent cation; and a is 1 or 2).

According to another aspect of the present disclosure, there is provided a resist pattern formation method, comprising a step of forming a resist film on a substrate by use of the aforementioned radiation-sensitive composition, a step of exposing the resist film to light, and a step of developing the light-exposed resist film.

According to another aspect of the present disclosure, there is provided a polymer including a structural unit represented by the aforementioned formula (1). Also, still another aspect of the present disclosure, there is provided a compound represented by the following formula (2)

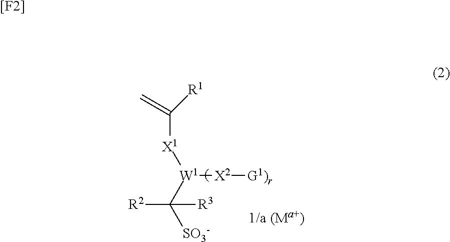

(wherein R¹ represents a hydrogen atom, a fluorine atom, a methyl group, or a trifluoromethyl group; X¹ represents a single bond, *¹—COO—, *¹—CONH—, or a divalent aromatic cyclic group; “*¹” represents a chemical bond with a carbon atom to which R¹ is bound; W¹ represents a (r+2)-valent organic group; X² represents *³—COO— or —O—; “*³” represents a chemical bond with W¹; G¹ represents an acid-releasable group; r is 1 or 2; when r is 2, two G¹s in the formula are identical to or different from each other; each of R² and R³ independently represents a fluorine atom or a fluoroalkyl group; M^{a+} represents an a-valent cation; and a is 1 or 2).

### Advantageous Effects of the Invention

By virtue of high sensitivity, the radiation-sensitive composition of the present disclosure can provide a suitable resist pattern by a small dose. In addition, since the radiation-sensitive composition of the present disclosure has excellent LWR performance, a wide process margin can be secured during pattern formation.

### MODES FOR CARRYING OUT THE INVENTION

### <<Radiation-Sensitive Composition>>

The radiation-sensitive composition of the present disclosure (hereinafter may also be referred to as “the present composition”) contains [A] a polymer. Also, the present composition may further contain, as optional components, one or more members from among [Z] an acid diffusion control agent, [B] a radiation-sensitive acid-generating agent (except for the polymer [A]), [D] a solvent, and [F] a high fluorine-content polymer. Hereinafter, the components will be described in detail.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is formed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure. The term “aromatic cyclic group” refers to an n-valent group formed by removing n (n is an integer of 1 or greater) hydrogen atom(s) from a ring moiety of a substituted or unsubstituted aromatic ring. The term “organic group” refers to an atomic group formed by removing any hydrogen atom from a carbon-containing compound (i.e., an organic compound) The term “aromatic ring” encompasses an aromatic hydrocarbon ring and an aromatic heterocycle.

The expression “substituted or unsubstituted p-valent hydrocarbon group (p is an integer of 1)” encompasses a p-valent hydrocarbon group (i.e., an unsubstituted p-valent hydrocarbon group) and a group formed by removing p hydrogen atoms from a hydrocarbon structure moiety of a hydrocarbon group having a substituent. Examples of the substituted or unsubstituted p-valent hydrocarbon group include an alkyl group and a fluoroalkyl group (corresponding to the case of p=1), and an alkanediyl group and a fluoroalkanediyl group (corresponding to the case of p=2). Among them, a fluoroalkyl group corresponds to a “substituted monovalent hydrocarbon group,” and a fluoroalkanediyl group corresponds to a “substituted divalent hydrocarbon group.” Such a feature similarly applies to other groups with the expression of “substituted or unsubstituted.” The term “(meth)acryl” is a term encompassing “acryl” and “methacryl.”

### <Polymer [A]>

The polymer [A] is a polymer including a structural unit represented by the following formula (1) (hereinafter may also be referred to as a “first structural unit”)

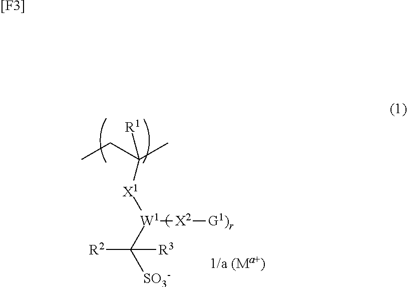

(In formula (1), R¹ represents a hydrogen atom, a fluorine atom, a methyl group, or a trifluoromethyl group; X¹ represents a single bond, *¹—COO—, *¹—CONH—, or a divalent aromatic cyclic group; “*¹” represents a chemical bond with a carbon atom to which R¹ is bound; W¹ represents a (r+2)-valent organic group; X² represents *³—COO— or —O—; “*,” represents a chemical bond with W¹; G¹ represents an acid-releasable group; r is 1 or 2; when r is 2, two G¹s in the formula are identical to or different from each other; each of R¹ and R³ independently represents a fluorine atom or a fluoroalkyl group; M^{a+} represents an a-valent cation; and a is 1 or 2.)

### (First Structural Unit)

In the aforementioned formula (1), R¹ is preferably a hydrogen atom or a methyl group, from the viewpoint of co-polymerizability of a monomer providing the first structural unit.

Examples of the divalent aromatic cyclic group represented by X¹ include a group formed by removing 2 hydrogen atoms from a ring moiety of a substituted or unsubstituted aromatic ring. The aromatic ring may be a monocycle or a condensed ring. Examples of the aromatic ring include a benzene ring, a naphthalene ring, an anthracene ring, and a phenanthrene ring. From the viewpoint of ease of synthesizing a monomer providing the first structural unit, the aromatic ring is preferably, among others, a benzene ring or a naphthalene ring, with a benzene ring being more preferred. When the divalent aromatic cyclic group represented by X¹ has a substituent, Examples of the substituent include a C1 to C5 alkyl group, a C1 to C5 alkoxy group, and a halogen atom (e.g., a fluorine atom, a chlorine atom, a bromine atom, or an iodine atom). No particular limitation is imposed on the number of substituents, and the number is, for example, 0 to 4, preferably 0 to 2.

G¹ is a group substituting a hydrogen atom of the carboxy group or the hydroxy group and is dissociated by the action of acid. When an acid is generated in the light-exposed part of the present composition through exposure to light, G¹ is dissociated by the generated acid and forms a carboxy group or a hydroxy group. As a result, conceivably, a change in solubility of the polymer [A] in a developer becomes considerable, whereby the contrast of the resist film formed from the present composition can be enhanced.

The group represented by “—X²-G¹” in the first structural unit preferably has an ester unit, when X² is —COO—. When X² is —O—, the group preferably has an ester group or an acetal group.

Examples of the acid-releasable group represented by G¹ include groups represented by the following formula (G-1):

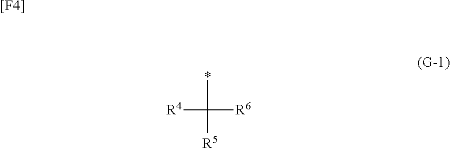

(wherein R⁴ represents a C1 to C20 substituted or unsubstituted monovalent hydrocarbon group; each of R¹ and R⁵ independently represents a C1 to C20 substituted or unsubstituted monovalent hydrocarbon group, or R⁵ and R⁶ taken together represent a C3 to C20 alicyclic hydrocarbon structure together with the carbon atom to which R⁵ and R⁶ bond; and “*” represents a chemical bond).

In the aforementioned formula (G-1), examples of the C1 to C20 monovalent hydrocarbon group represented by R⁴, R⁵, or R⁶ include a C1 to C20 monovalent chain hydrocarbon group, a C3 to C20 monovalent alicyclic hydrocarbon group, and a C1 to C20 monovalent aromatic hydrocarbon group.

Examples of the C1 to C20 monovalent chain hydrocarbon group include alkyl groups such as methyl, ethyl, n-propyl, i-propyl, n-butyl, and t-butyl; alkenyl groups such as ethenyl, propenyl, and butenyl; and alkynyl groups such as ethynyl, propynyl, and butynyl. Among them, the C1 to C20 monovalent chain hydrocarbon group represented by R⁴, R⁵, or R⁶ is preferably an alkyl group, more preferably a C1 to C4 alkyl group.

Examples of the C3 to C20 monovalent alicyclic hydrocarbon group include monovalent monocyclic alicyclic saturated hydrocarbon groups such as cyclopentyl, cyclohexyl methylcyclopentyl, ethylcyclopentyl, methylcyclohexyl, and ethylcyclohexyl; monovalent monocyclic alicyclic unsaturated hydrocarbon groups such as cyclopentenyl, cyclohexenyl, methylcyclopentenyl, and methylcyclohexenyl; monovalent polycyclic alicyclic saturated hydrocarbon groups such as norbornyl, adamantyl, and tricyclodecyl; and monovalent polycyclic alicyclic unsaturated hydrocarbon groups such as norbornenyl, tricyclodecenyl, and indanyl.

Examples of the C6 to C20 monovalent aromatic hydrocarbon group include aryl groups such as phenyl, tolyl, xylyl, mesityl, naphthyl, methylnaphthyl, anthryl, methylanthryl, and indenyl; and aralkyl groups such as benzyl, phenethyl, naphtylmethyl, and anthrylmethyl.

Examples of the C3 to C20 alicyclic hydrocarbon structure formed by combining R⁵ and R⁶ together with the carbon atom to which R⁵ and R⁶ bond include monocyclic saturated alicyclic hydrocarbon structures such as a cyclopropane structure, a cyclobutane structure, a cyclopentane structure, a cyclohexane structure, a cycloheptane structure, a cyclooctane structure; monocyclic unsaturated alicyclic hydrocarbon structures such as a cyclopentene structure and a cyclohexene structure; and polycyclic alicyclic hydrocarbon structures such as a norbornane structure, an adamantane structure, a tricyclodecane structure, and a tetracyclododecane structure.

When the group represented by R⁴, R⁵, or R⁶ has a substituent, examples of the substituent include a halogen atom (e.g., a fluorine atom, a bromine atom, a chlorine atom, or an iodine atom), a hydroxy group, and a C1 to C3 alkoxy group. When R⁵ and R⁶ taken together with the carbon atom to which R⁵ and R⁶ bond to thereby form the C3 to C20 alicyclic hydrocarbon structure, any of the above-exemplified substituent or an alkyl group may bound to the ring.

Specific examples of the group represented by “—COO-G¹” in the first structural unit include groups represented by the following formulas.

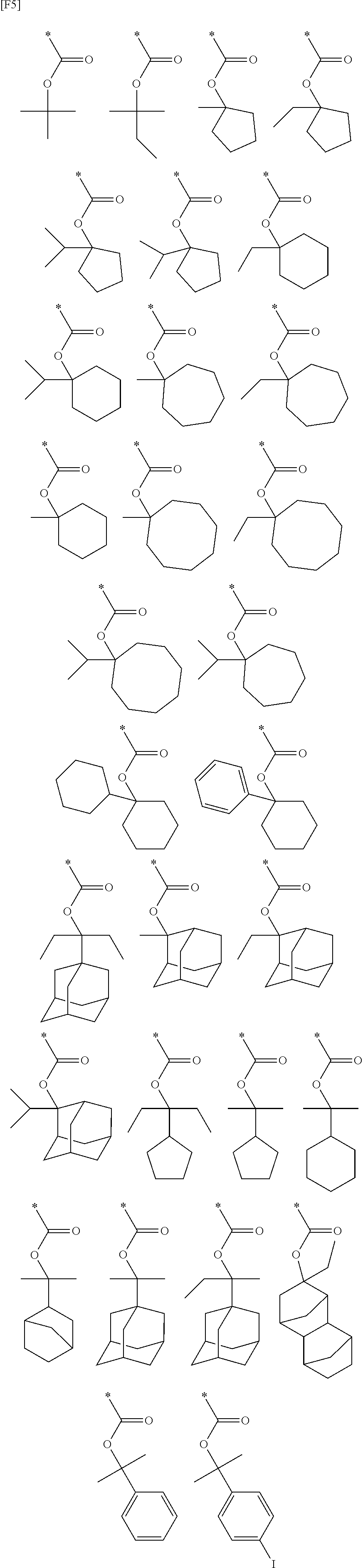

(In the formulas, “*” represents a chemical bond.)

Specific examples of the group represented by “—O-G¹” in the first structural unit include groups represented by the following formulas.

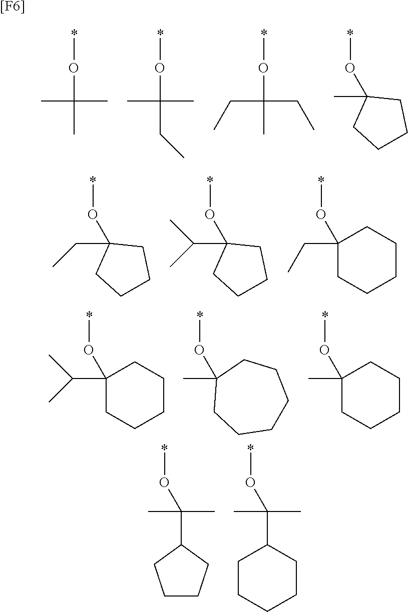

(In the formulas, “*” represents a chemical bond.)

When the X² in the aforementioned formula (1) is —O—, examples of the acid-releasable group represented by G¹ include, in addition to groups represented by the aforementioned formula (G-1), groups represented by the following formula (G-2)

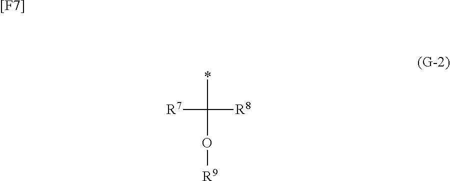

(In formula (G-2), R⁷ represents a hydrogen atom or a C1 to C20 substituted or unsubstituted monovalent hydrocarbon group. Regarding R⁸ and R⁹, in one possible case, R⁸ is a hydrogen atom or a C1 to C20 substituted or unsubstituted monovalent hydrocarbon group, and R⁹ is a C1 to C20 substituted or unsubstituted monovalent hydrocarbon group. In an alternative case, R⁸ and R⁹ taken together represent a ring together with the oxygen atom to which R⁵ bonds and the oxygen atom to which R⁹ bonds. “*” represents a chemical bond).

In the aforementioned formula (G-2), examples of the monovalent hydrocarbon group represented by R⁷, R⁸, or R⁹ include a C1 to C20 monovalent chain hydrocarbon group, a C3 to C20 monovalent alicyclic hydrocarbon group, and a C6 to C20 monovalent aromatic hydrocarbon group. Specific examples thereof include the same groups as exemplified in relation to the description of R⁴, R⁵, and R⁶ of the aforementioned formula (G-1). When R⁷, R⁸, or R⁹ has a substituent, examples of the substituent include a halogen atom (e.g., a fluorine atom, a chlorine atom, a bromine atom, or an iodine atom), a hydroxy group, and a C1 to C3 alkoxy group.

In the group represented by “—O-G¹” in the first structural unit, when G¹ is a group represented by the aforementioned formula (G-2), specific examples include the groups represented by the following formulas.

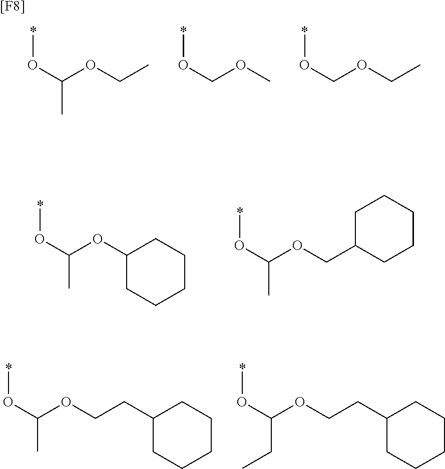

(In the formulas, “*” represents a chemical bond.)

Examples of the (r+2)-valent organic group represented by W¹ include a substituted or unsubstituted C1 to C30 hydrocarbon group, a C1 to C30 group in which a methylene group in the hydrocarbon group is replaced by a hetero atom-containing group (e.g., —O—, —S—, —CO—, —COO—, —NH—, —NHCO—, or —SO₂—) (hereinafter may also be denoted as a “group R^{B}”) and a group formed by substituting at least one hydrogen atom of the group R^{B}. Examples of the substituent of the substituted hydrocarbon group or that of the substituted group R^{B} include a halogen atom (e.g., a fluorine atom, a chlorine atom, a bromine atom, or an iodine atom), a hydroxy group, an aldehyde group, an acetyl group, and an ester group.

In the (r+2)-valent organic group represented by W¹, the group represented by “—X²-G¹” may be bound to a chain structure of W¹ or to a ring of W¹. When the group represented by “—X-G¹” is bound to the chain structure, W¹ preferably has a partial structure in which the group represented by “—X²-G¹” is bound to a carbon atom of the chain structure. When the group represented by “—X²-G¹” is bound to a carbon atom of the chain structure, the group represented by “—X²-G¹” may substitute a hydrogen atom of a C≥1 alkanediyl group or a hydrogen atom of a C≥1 alkyl group.

When the group represented by “—X²-G¹” is bound to a ring of W¹, examples of the ring to which the group represented by “—X²-G¹” is bound include a C3 to C20 aliphatic hydrocarbon ring, a C3 to C20 aliphatic heterocycle, a C6 to C20 aromatic hydrocarbon ring, and a C5 to C20 aromatic heterocycle. These rings may have a substituent. Examples of the substituent include an alkoxy group, an alkoxycarbonyl group, a halogen atom (e.g., a fluorine atom, a chlorine atom, a bromine atom, or an iodine atom), a hydroxy group, and a cyano group.

The a C3 to C20 aliphatic hydrocarbon ring may be a monocycle or polycycle, or a saturated hydrocarbon ring or an unsaturated hydrocarbon ring. Examples of the saturated hydrocarbon ring of the monocycle aliphatic hydrocarbon ring include a cyclopentane ring, a cyclohexane ring, a cycloheptane ring, and a cyclooctane ring. Examples of the unsaturated hydrocarbon ring of the monocycle aliphatic hydrocarbon ring include a cyclopentene ring, a cyclohexene ring, a cycloheptene ring, a cyclooctene ring, and a cyclodecene ring. The polycyclic aliphatic hydrocarbon ring may be a bridged alicyclic hydrocarbon structure or a condensed alicyclic hydrocarbon structure. Preferably, the polycyclic aliphatic hydrocarbon ring is a bridged alicyclic saturated hydrocarbon ring and has a bicyclo[2.2.1]heptane structure, a bicyclo[2.2.2]octane structure, or a tricyclo[3.3.1.1^{3,7}]decane structure.

Examples of the C3 to C20 aliphatic heterocycle include a ring having a cyclic ether structure, a cyclic acetal structure, a lactone structure, a cyclic carbonate structure, a sultone structure, or a thioxane structure. The aliphatic heterocycle may be a monocycle or a polycycle or may have any of a bridged structure, a condensed cyclic structure, and a spiro ring structure. Also, the aliphatic heterocycle may be a combination of two or more of a bridged structure, a condensed ring structure, and a spiro ring structure.

Examples of the C6 to C20 aromatic hydrocarbon ring include a benzene ring, a naphthalene ring, an anthracene ring, an indene ring, and a fluorene ring. Examples of the C5 to C20 aromatic heterocycle include a furan ring and thiophene ring.

From the viewpoint of enhancement of the hydrophobicity of the resist film formed from the present composition, to achieve a greater difference between solubility in a developer between the light-exposed part and the light-unexposed part, W¹ preferably has a cyclic structure. More preferably, W¹ has at least one ring selected from the group consisting of an aromatic ring, an aliphatic hydrocarbon ring, and an aliphatic heterocycle, and a partial structure in which a group represented by “—X²-G¹” is bound to the ring, or has a chain structure to which a group represented by “—X²-G³” is bound, and has at least one ring selected from the group consisting of an aromatic hydrocarbon ring, an aliphatic hydrocarbon ring, and an aliphatic heterocycle. When W¹ has a partial structure in which the chain structure to which a group represented by “—X²-G¹” is bound, examples of the ring which W¹ has include the same rings as exemplified as specific examples in relation to the case where a group represented by “—X²-G¹” is bound to the ring.

From the viewpoint of ease of synthesis, r is preferably 1.

Specific examples of the divalent group in which the group represented by “—X²-G¹” is bound to W¹ in the aforementioned formula (1); i.e., a divalent group represented by the following formula:

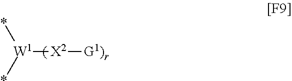

(wherein “*” represents a chemical bond) include a group represented by the following formula (w-1).

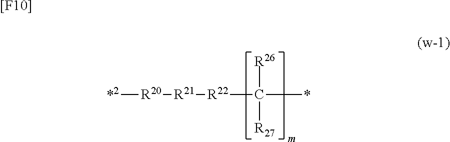

(In formula (w-1), R²⁰ represents a single bond or a divalent linking group; R²¹ represents a divalent group represented by the following formula (r3-1) or (r3-2); R²² represents a divalent linking group; each of R²⁶ and R²⁷ independently represents a hydrogen atom, a fluorine atom, a C1 to C3 alkyl group, or a C1 to C3 fluoroalkyl group; m is 0 or 1; “*” represents a chemical bond in the SO₃⁻ side; and “*²” represents a chemical bond on the X¹ side.)

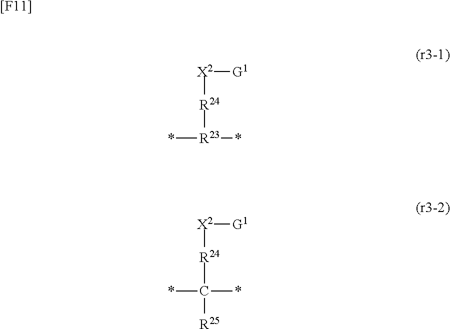

(In formulas (r3-1) and (r3-2), R²³ represents a trivalent aromatic hydrocarbon ring group, a trivalent aliphatic hydrocarbon ring group, or a trivalent aliphatic heterocyclic group; R²⁴ represents a single bond or a C1 to C3 alkanediyl group; R²⁵ represents a hydrogen atom or a C1 to C3 alkyl group; X² represents *⁴—COO— or —O—; “*⁴” represents a chemical bond with R²⁴; G¹ represents an acid-releasable group; and “*” represents a chemical bond.)

In the aforementioned formula (w-1), examples of the divalent linking group represented by R²⁰ or R²² include a C1 to C20 group, —COO—, and —O—, among the groups as exemplified in relation to the (r+2)-valent organic group represented by W¹. From the viewpoint of sensitivity, R²⁰ preferably has no fluorine atom. When R²¹ is a group represented by the aforementioned formula (r3-2), R²² preferably has at least one ring selected from the group consisting of an aromatic ring, an aliphatic ring, and a heterocycle.

In the aforementioned formulas (r3-1) and (r3-2), specific examples of the ring which the trivalent aromatic hydrocarbon ring group, an aliphatic hydrocarbon ring group, or an aliphatic heterocycle group represented by R²³ include the same rings as exemplified as specific examples in relation to the case where a group represented by “—X²-G¹” is bound to the ring in W¹. Specific examples of G¹ include a group represented by the aforementioned formula (G-1). R²⁴ is preferably a single bond or a methylene group. R²⁵ is preferably a hydrogen atom or a methyl group.

In the aforementioned formula (1), the fluoroalkyl group represented by R² or R³ is preferably a C1 to C10 group, and examples thereof include trifluoromethyl, 2,2,2-trifluoroethyl, perfluoroethyl, 2,2,3,3,3-pentafluoropropyl, 2,2,2-trifluoro-1-(trifluoromethyl)ethyl, perfluoro-n-propyl, perfluoroisopropyl, perfluoro-n-butyl, perfluoroisobutyl, perfluoro-t-butyl, 2,2,3,3,4,4,5,5-octafluoropentyl, and perfluorohexyl. Of these, a C1 to C5 is preferred, with trifluoromethyl, 2,2,2-trifluoroethyl, or perfluoroethyl being more preferred, trifluoromethyl being still more preferred.

From the viewpoint of sensitivity, R² and R³ are preferably a fluorine atom, a trifluoromethyl group, a 2,2,2-trifluoroethyl group, or a perfluoroethyl group, with a fluorine atom or a trifluoromethyl group being more preferred.

The a-valent cation represented by M^{a+} is preferably an organic cation. From the viewpoint of achieving favorable lithographic characteristics of the present composition, M^{a+} is preferably a sulfonium cation, an iodonium cation, or an ammonium cation, more preferably a sulfonium cation or an iodonium cation.

in the case where “a” in formula (1) is 1, specific examples of M^{a+} include a cation represented by formula (7), a cation represented by formula (8), and a cation represented by formula (9).

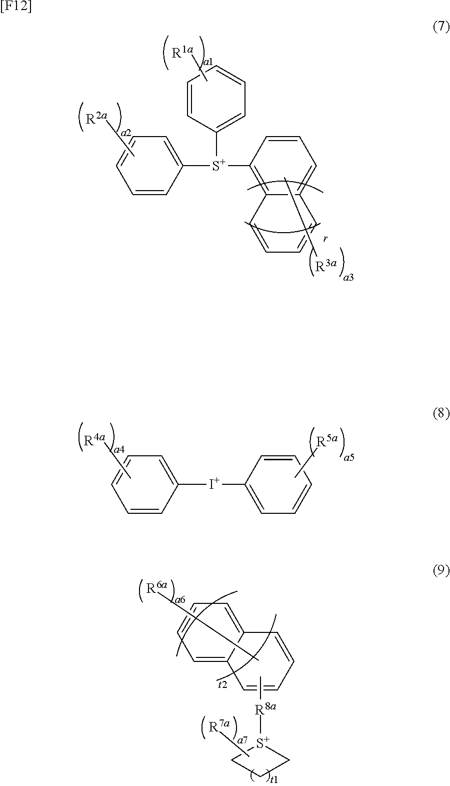

(In formula (7), each of Ria and R^{2a} represents a monovalent substituent, or R^{1a} and R^{2a} taken together represent a single bond or a divalent group connecting a ring to which Ria bonds and a ring to which R^{1a} bonds; R^{3a} represents a monovalent substituent; each of a1 and a2 is an integer of 0 to 5; a3 is an integer of 0 to (2×r+5); and r is 0 or 1;

in formula (8), each of R=a and R^{5a} represents a monovalent substituent. Each of a4 and a5 is an integer of 0 to 5; and

in formula (9), a6 is an integer of 0 to 7. When a6 is 1, R^{6a} is a C1 to C20 monovalent organic group, a hydroxy group, a nitro group, or a halogen group. When a6 is 2 or greater, a plurality of R^{6a}s, which are identical to or different from one another, represents a C1 to C20 monovalent organic group, a hydroxy group, a nitro group, or a halogen group, or two of the plurality of R^{6a}s taken together represent a 4- to 20-membered ring together with the carbon atom to which two of the plurality of R^{6a}s bond. The number a7 is an integer of 0 to 6. When a7 is 1, R^{7a} is a C1 to C20 monovalent organic group, a hydroxy group, a nitro group, or a halogen group. When a7 is 2 or greater, a plurality of R^{7a}s, which are identical to or different from one another, represents a C1 to C20 monovalent organic group, a hydroxy group, a nitro group, or a halogen group, or two of the plurality of R^{7a}s taken together represent a 3- to 20-membered ring together with the carbon atom to which two of the plurality of R^{7a}s bond. The number t1 is an integer of 0 to 3. R^{8a} represents a single bond or a C1 to C20 divalent organic group. The number t2 is 0 or 1.)

In formulas (7) and (8), examples of the monovalent substituent represented by R^{1a}, R^{2a}, R^{3a}, R^{4a}, or R^{5a} (hereinafter denoted by “R^{1a} to R^{5a}”) include a fluorine atom, a chlorine atom, a bromine atom, an iodine atom, a substituted or unsubstituted alkyl group, a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

The alkyl group represented by any of R^{1a} to R^{5a} may be linear-chain or branched. The alkyl group is preferably a C1 to C10. Of these, the alkyl group represented by any of R^{1a} to R^{7a} is preferably a C1 to C5, with methyl, ethyl, n-butyl, and t-butyl being more preferred. When any of R^{1a} to R^{5a} is an alkoxy group, specific examples thereof include a group in which any of the above-exemplified alkyl groups is attached to the alkyl moiety forming the alkoxy group. The alkoxy group is preferably methoxy, ethoxy, n-propoxy, or n-butoxy.

The cycloalkyl group represented by any of R^{1a} to R^{5a} may be monocyclic or polycyclic. Examples of the monocyclic cycloalkyl group include cyclopropyl, cyclobutyl, cyclopentyl, cyclohexyl, and cyclooctyl. Examples of the polycyclic cycloalkyl group include norbornyl, adamantyl, tricyclodecyl, and tetracyclododecyl. When any of Ra to Ra is a cycloalkyloxy group, specific examples thereof include a group in which any of the above-exemplified cycloalkyl groups is attached to the cycloalkyl moiety forming the cycloalkyloxy group. The cycloalkyloxy group represented by any of R^{1a} to R^{5a} is preferably a cyclopentyloxy group or a cyclohexyloxy group.

When the cycloalkyl group represented by any of R^{1a} to R^{5a} has a substituent, examples of the substituent include a fluorine atom, a chlorine atom, a bromine atom, an iodine atom, a hydroxy group, a carboxy group, a cyano group, a nitro group, and a C1 to C5 alkoxy group.

When any of R^{1a} to R^{5a} is an ester group (—COOR), specific examples of the hydrocarbon moiety (R) of the ester group include the above-exemplified, substituted or unsubstituted alkyl groups and substituted or unsubstituted cycloalkyl groups. Among them, when any of R^{1a} to R^{5a} is an ester group, R^{1a} to R^{5a} are preferably methoxycarbonyl ethoxycarbonyl, or n-butoxycarbonyl. When any of R^{1a} to R^{5a} is an alkylsulfonyl group, examples of the alkyl moiety forming the alkylsulfonium group include the above-mentioned substituted or unsubstituted alkyl groups. When any of R^{1a} to R^{5a} is a cycloalkylsulfonyl group, examples of the alkyl moiety forming the cycloalkylsulfonium group include the aforementioned, substituted or unsubstituted cycloalkyl groups.

When R^{1a} and R^{2a} taken together represent a divalent group connecting a ring to which R^{1a} bonds and a ring to which R^{1a} bonds, examples of the divalent group include —COO—, —OCO—, —CO—, —O—, —SO—, —SO₂—, —S—, a C1 to C3 alkanediyl group, a C2 or C3 alkenediyl group, and a group in which includes—O—, —S—, —COO—, —OCO—, —CO—, —SO—, or —SO₂-intervening between the C—C bond of an ethylene group. Among them, R^{1a} and R^{2a} are preferably a single bond linking ring structures or form—O— or —S—.

The number a1 is preferably an integer of 0 to 2. When a1 is 1 or greater, at least one of the R^{1a}s in the aforementioned formula (7) is preferably a fluorine atom, an iodine atom, or a trifluoromethyl group. The number a2 is preferably an integer of 0 to 2. When a2 is 1 or greater, at least one of R^{2a}s in the aforementioned formula (7) is preferably a fluorine atom, an iodine atom, or a trifluoromethyl group. The number a3 is preferably an integer of 0 to 2. When a3 is 1 or greater, at least one of Ras in formula (7) is preferably a fluorine atom, an iodine atom, or a trifluoromethyl group. Particularly, all of a1, a2, and a3 are independently and preferably an integer of 0 to 2. More preferably, all of a1, a2, and a3 are independently an integer of 1 or 2, and at least one R^{1a}, at least one R^{2a}, or at least one R^{3a} in the aforementioned formula (7) is a fluorine atom, an iodine atom, or a trifluoromethyl group.

The number a4 is preferably an integer of 0 to 2. When a4 is 1 or greater, at least one of the R^{4a}s in the aforementioned formula (8) is preferably a fluorine atom, an iodine atom, or a trifluoromethyl group. The number a5 is preferably an integer of 0 to 2. When a5 is 1 or greater, at least one of the R^{5a}S in the aforementioned formula (8) is preferably a fluorine atom, an iodine atom, or a trifluoromethyl group. Particularly, each of a4 and a5 is independently and preferably an integer of 0 to 2. More preferably, each of a4 and a5 is independently an integer of 1 or 2, and at least one R^{4a} and at least one R^{4a} in the aforementioned formula (8) is a fluorine atom, an iodine atom, or a trifluoromethyl group.

In the aforementioned formula (9), examples of the C1 to C20 monovalent organic group represented by R^{6a} or R^{7a} include a substituted or unsubstituted C1 to C20 monovalent hydrocarbon group, —OR^{k}, —COOR^{k}, —O—CO—CR^{k}, —OR^{kk}—CO—R^{k}, —OSO₂—R^{k}, or —SO₂—R^{k}. R^{k} represents a C1 to C10 monovalent hydrocarbon group. R^{kk} represents a single bond or a C1 to C10 divalent hydrocarbon group. Examples of the C1 to C20 monovalent hydrocarbon group include the same groups as exemplified in relation to monovalent hydrocarbon groups represented by R⁴ to R⁶ in the aforementioned formula (G-1). Also, in R^{6a} and R^{7a}, examples of the substituent which replaces a hydrogen atom of a hydrocarbon group include the same groups as exemplified in relation to substituents included in the aforementioned R^{1a} to R^{5a}.

Examples of the divalent organic group represented by R^{8a} include a group formed by removing one hydrogen atom from a C1 to C20 monovalent organic group exemplified as R^{6a} or R^{7a}.

Among the aforementioned examples, each of R^{6a} and R^{7a} is preferably a linear-chain or branched monovalent alkyl group or monovalent fluoroalkyl group, a monovalent aromatic hydrocarbon group, —OSO₂—R^{k}, or —SO₂—R^{k}. The number a6 is preferably an integer of 0 to 2, more preferably 0 or 1. The number a7 is preferably an integer of 0 to 2, more preferably 0 or 1. The number t2 is preferably 0. The number t1 is preferably 2 or 3.

In the aforementioned formula (1), a is preferably 1.

Specific examples M^{a+} include cations represented by the following formulas. However, M^{a+} is not limited thereto.

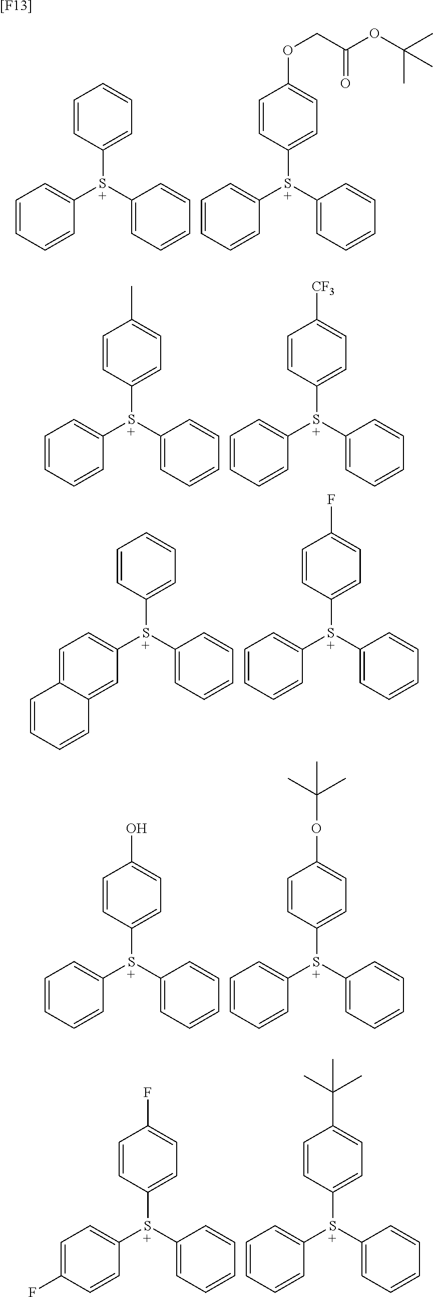

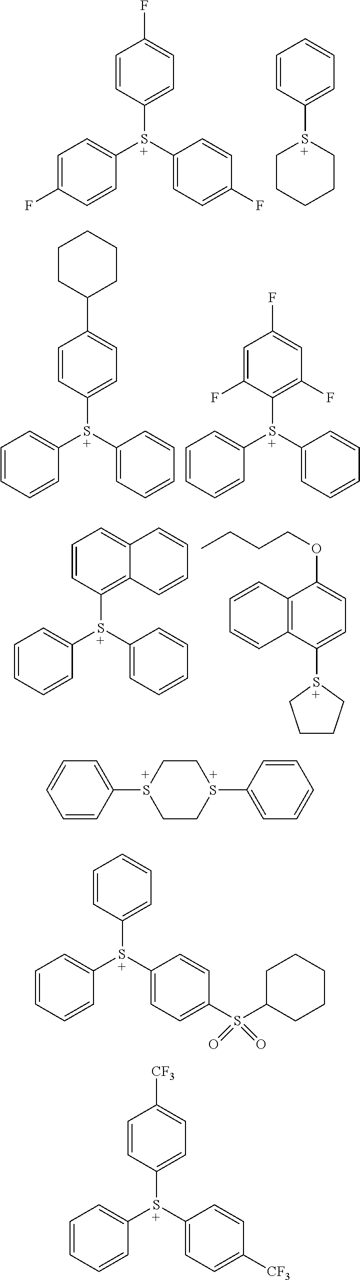

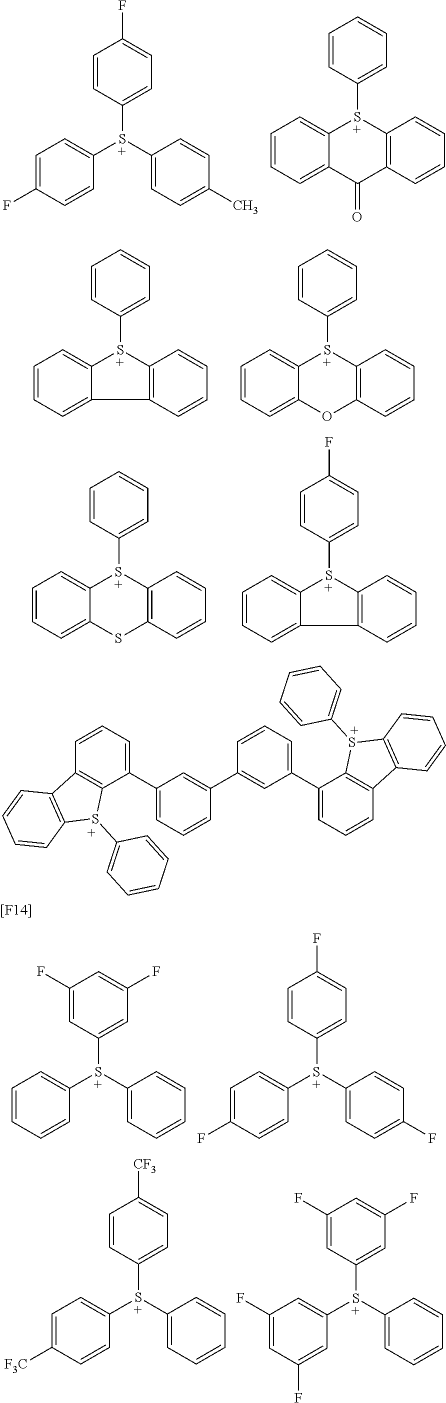

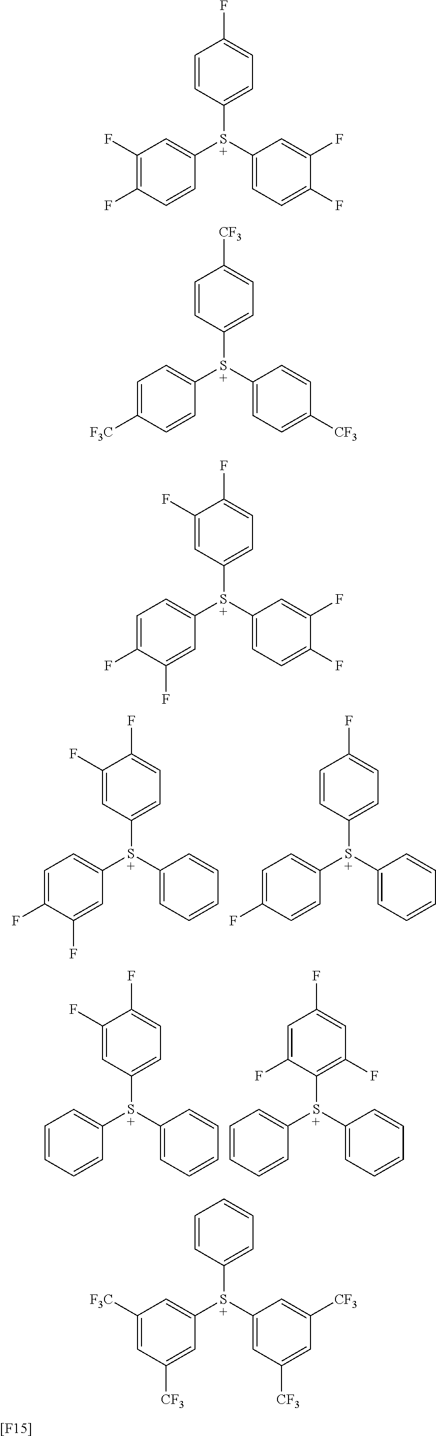

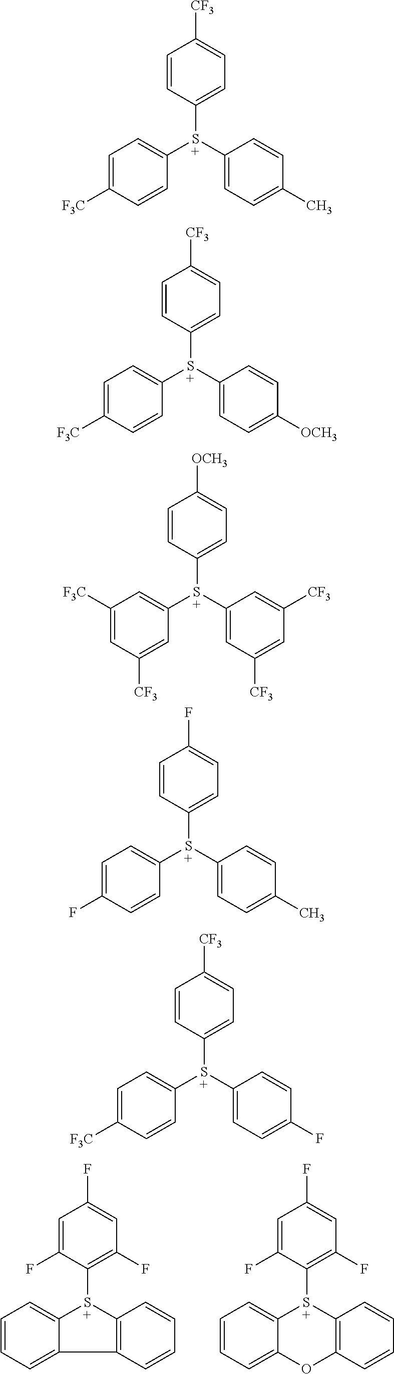

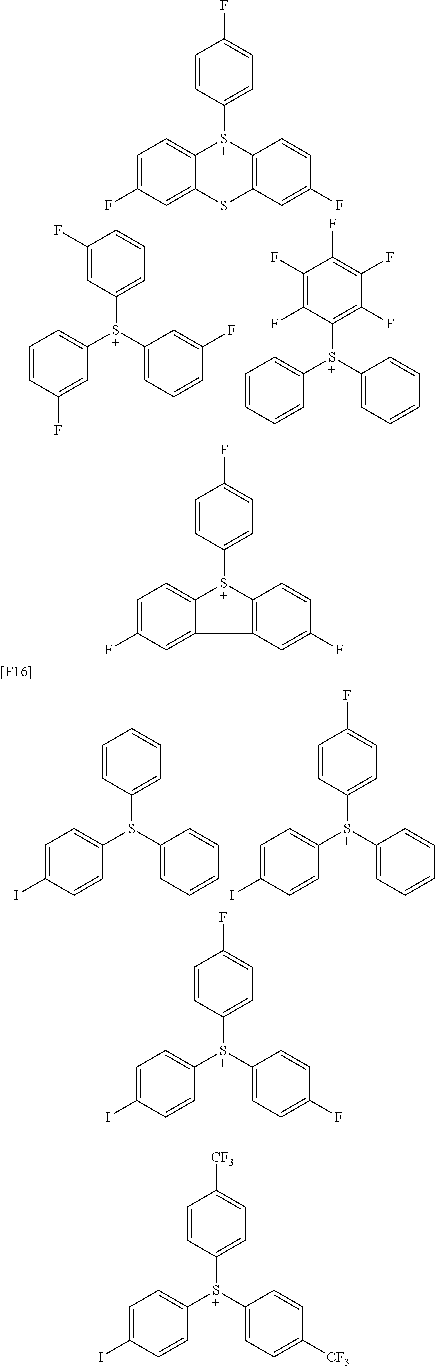

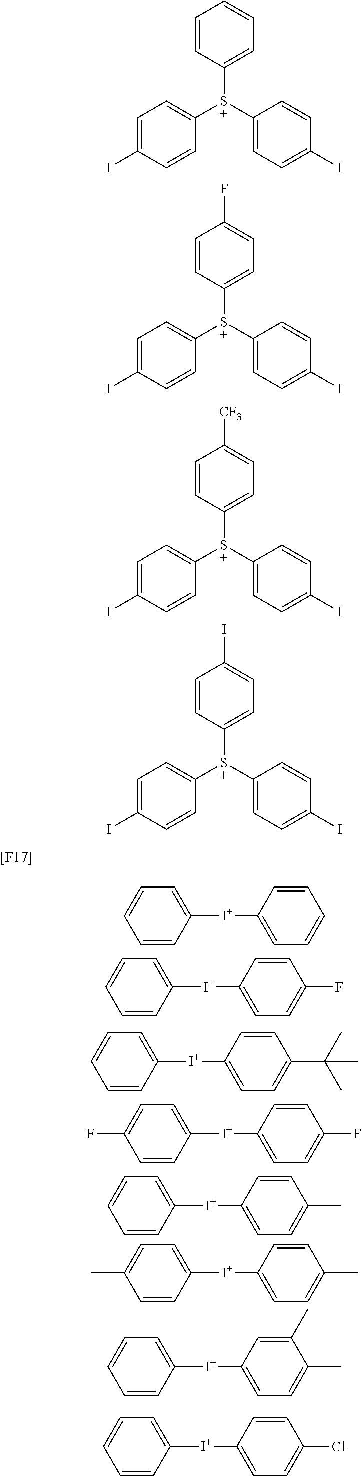

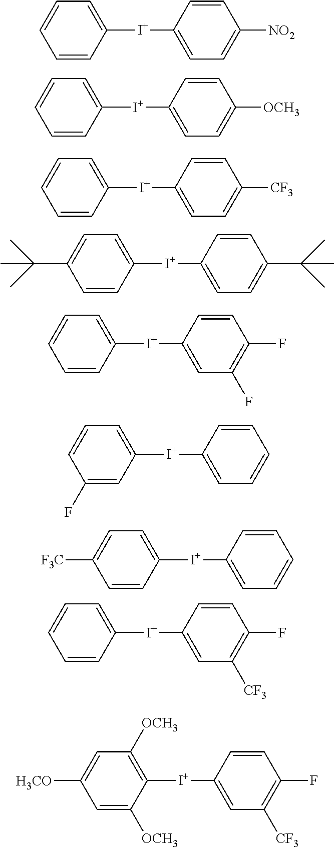

Specific examples of the first structural unit include structural units represented by the following formulas. However, the first structural unit is not limited to the following specific examples.

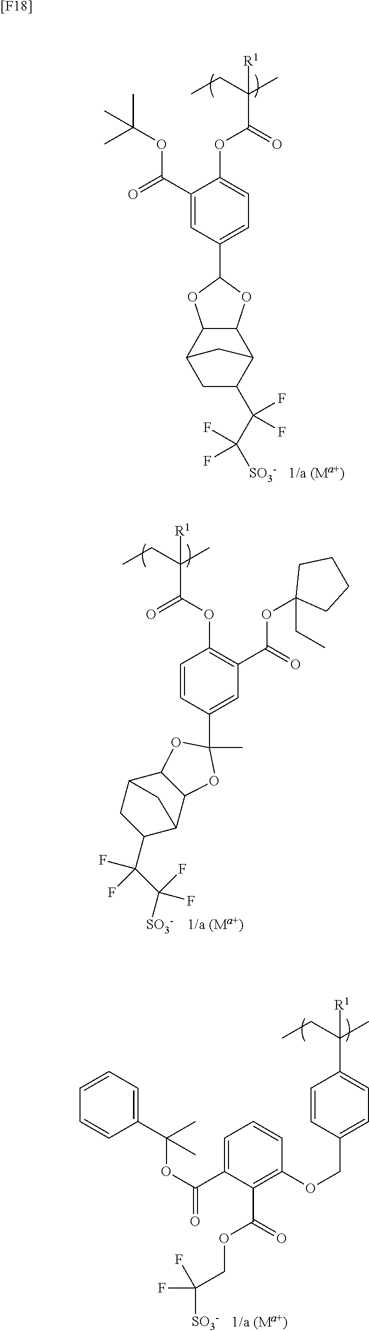

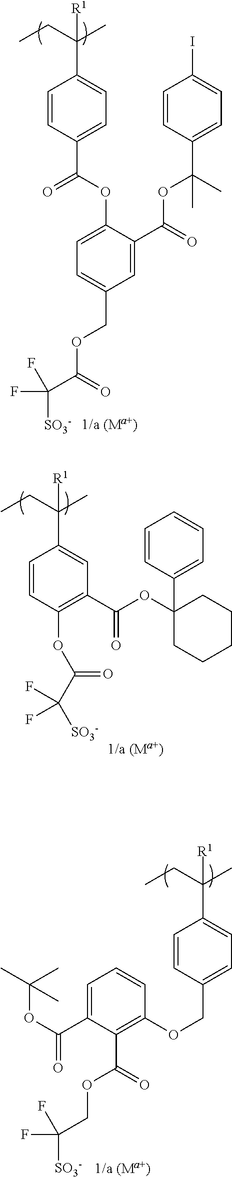

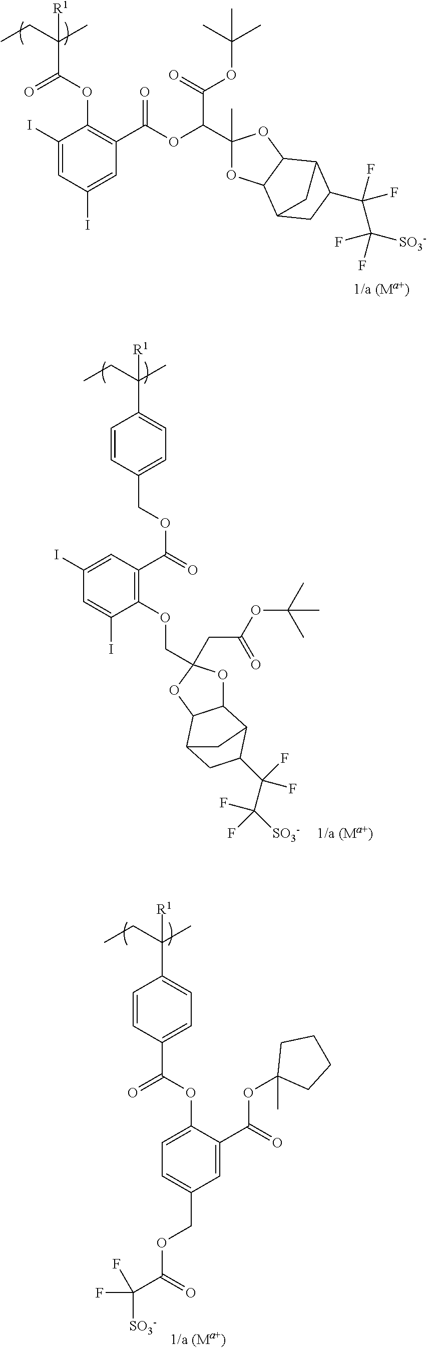

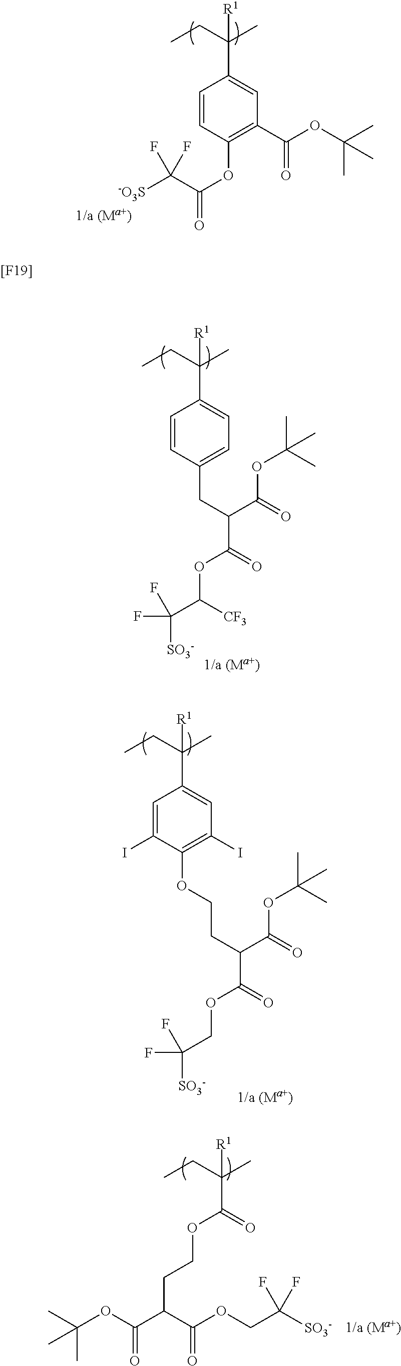

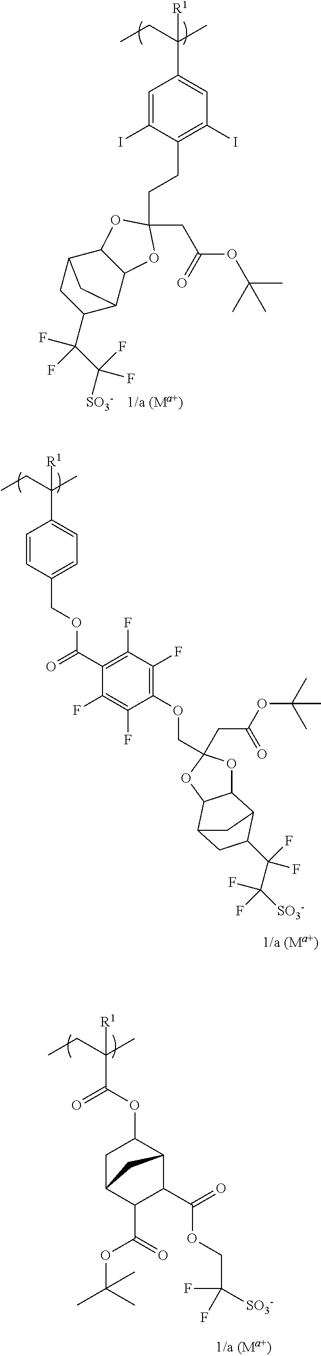

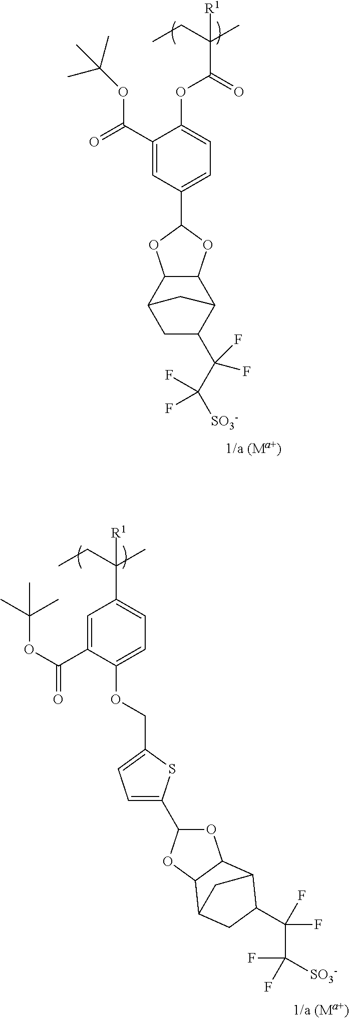

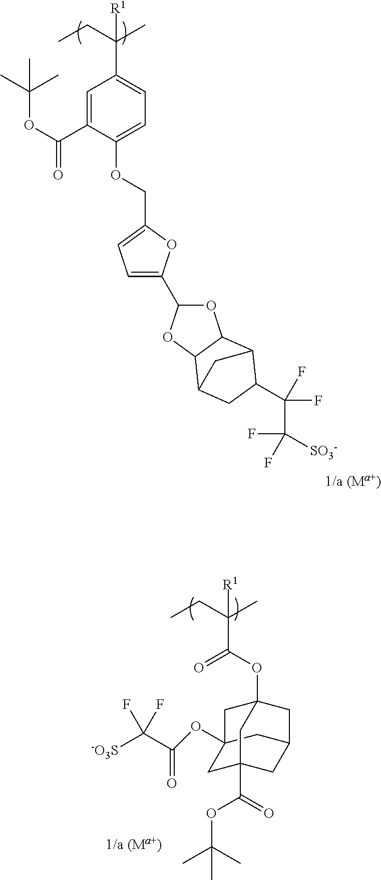

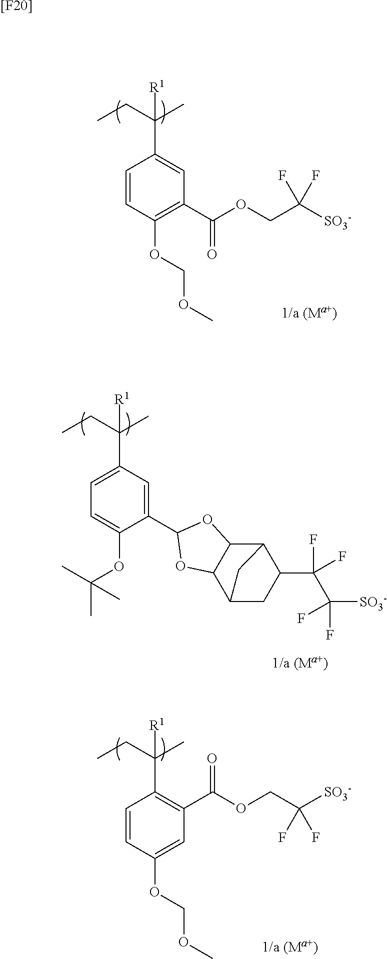

(In the above formulas, M^{a+} represents an a-valent cation, and a is 1 or 2.)

In the polymer [A], the relative amount of the first structural unit, with respect to all the structural units forming the polymer [A], is preferably 1 mol % or more, more preferably 2 mol % or more, still more preferably 5 mol % or more. Also, the relative amount of the first structural unit, with respect to all the structural units forming the polymer [A], is preferably 50 mol % or less, more preferably 40 mol % or less, still more preferably 30 mol % or less. By adjusting the first structural unit content to satisfy the above conditions, the sensitivity of the present composition is enhanced, and the difference in solubility in developer between the light-exposed part and the light-unexposed part (i.e., dissolution contrast) can be enhanced. Thus, the process margin during pattern formation can be further widened, which is preferred.

### (Additional Structural Unit)

In addition to the first structural unit, the polymer [A] may further include a structural unit other than the first structural unit (hereinafter may also be referred to as an “additional structural unit”). Examples of the additional structural unit include second to fifth structural units shown below.

### Second Structural Unit

The polymer [A] may further include a structural unit having an aromatic ring and a hydroxy group bound to the aromatic ring (except for a structural unit corresponding to the first structural unit) (hereinafter referred to as a “second structural unit”). Incorporation of the second structural unit into the polymer [A] is preferred, since lithographic characteristics of the present composition (e.g., LWR performance and critical dimension uniformity (CDU) can be further enhanced, and the effect of suppressing dissolution of a light-unexposed part of the polymer in a developer is high, thereby sufficiently reducing development failure. Particularly when pattern formation is conducted through exposure to a radiation having a wavelength of 50 nm or less (e.g., an electron beam or EUV), a polymer including a second structural unit is preferably applied. When pattern formation is conducted through exposure to a radiation having a wavelength of 50 nm or less, the polymer [A] preferably includes the second structural unit.

Examples of the aromatic ring to which a hydroxy group is bonded include a benzene ring, a naphthalene ring, an anthracene ring, and a phenanthrene ring. Of these, a benzene ring or a naphthalene ring is preferred, with a benzene ring being more preferred. In the second structural unit, no particular limitation is imposed on the number of the hydroxy group(s) bonded to the aromatic ring, and the number is preferably 1 to 3, more preferably 1 or 2.

Examples of the second structural unit include structural units represented by the following formula (ii)

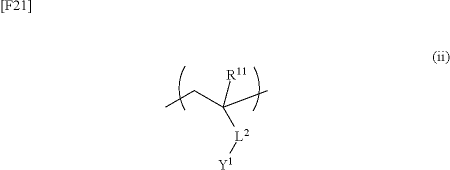

(In formula (ii), R¹¹ represents a hydrogen atom, a fluoro group, a methyl group, or a trifluoromethyl group; L² represents a single bond, an ether bond, a carbonyl group, an ester bond, or an amide bond; and Y¹ represents a monovalent group having a hydroxy group which is bonded to an aromatic ring.)

In the aforementioned formula (ii), from the viewpoint of copolymerizability of the monomer forming the second structural unit, R¹¹ is preferably a hydrogen atom or a methyl group. L² is preferably a single bond or an ester bond.

Specific examples of the second structural unit include structural units represented by the following formulas.

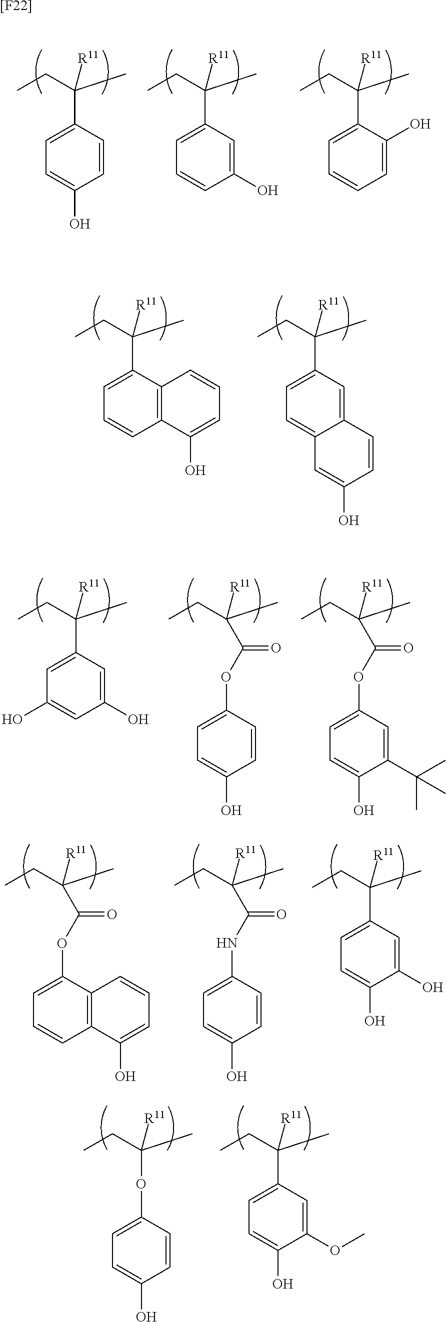

(In the above formulas, R¹¹ represents a hydrogen atom, a fluoro group, a methyl group, or a trifluoromethyl group.)

In the polymer [A], the relative amount of the second structural unit in all the structural units forming the polymer [A] is preferably 5 mol % or more, more preferably 10 mol % or more, still more preferably 15 mol % or more. Also, the relative amount of the second structural unit in all the structural units forming the polymer [A] is preferably 90 mol % or less, more preferably 80 mol % or less, still more preferably 60 mol % or less. Adjusting the second structural unit content to satisfy the aforementioned conditions is preferred, since the lithographic characteristics of the present composition can be further enhanced.

Notably, in addition to the polymer having the first structural unit, the polymer [A] may include a polymer having the second structural unit. From the viewpoint of yielding a radiation-sensitive composition having excellent lithographic characteristics (e.g., LWR (line width roughness) performance and CDU performance) and failure suppression property, the present composition preferably contains, as the polymer [A], a polymer including the first structural unit and the second structural unit in the same molecule.

### Third Structural Unit

The polymer [A] may further include a structural unit having an acid-releasable group and no onium salt structure (hereinafter referred to as a “third structural unit”). The acid-releasable group of the third structural unit is preferably a group which can substitute a hydrogen atom of an acid group such as a carboxy group or a hydroxy group and which is released by the action of acid. Through further incorporation of the third structural unit into the polymer [A], the present composition can be provided with suitable lithographic characteristics in the following manner. Specifically, when the present composition is exposed to light, an acid is generated. The acid-releasable group in the third structural unit is released by the generated acid, to thereby form an acid group. The solubility of the polymer [A] in a developer is modified by the acid group, whereby the present composition can be provided with suitable lithographic characteristics.

Examples of the third structural unit include a structural unit represented by the following formula (iii-1) (hereinafter may also be referred to as a “structural unit (III-1)”), a structural unit represented by the following formula (iii-2) (hereinafter may also be referred to as a “structural unit (III-2)”), and a structural unit represented by the following formula (iii-3) (hereinafter may also be referred to as a “structural unit (III-3)”).

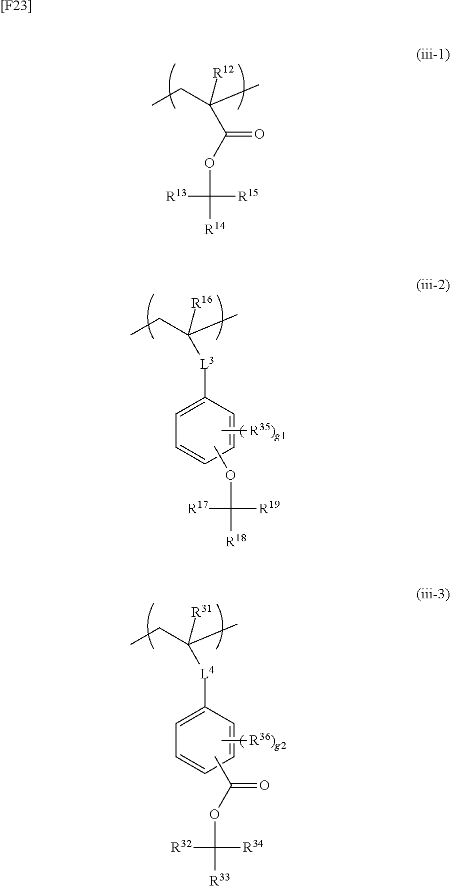

(In formula (iii-1), R¹² represents a hydrogen atom, fluorine atom, a methyl group, or a trifluoromethyl group; R¹³ represents a hydrogen atom or a C1 to C20 substituted or unsubstituted monovalent hydrocarbon group; each of R¹⁴ and R¹⁵ independently represents a C1 to C20 substituted or unsubstituted monovalent hydrocarbon group or aromatic heterocyclic group, or R¹⁴ and R¹⁵ taken together represent a C3 to C20 alicyclic hydrocarbon structure together with the carbon atom to which R¹⁴ and R¹⁵ bond; when R¹³ is a hydrogen atom, one or both of R¹⁴ and R¹⁵ are a monovalent unsaturated hydrocarbon group or aromatic heterocyclic group, or R¹⁴ and R¹⁵ taken together represent a C3 to C20 unsaturated alicyclic hydrocarbon structure together with the carbon atom to which R¹⁴ and R¹⁵ bond;

in formula (iii-2), R¹⁶ represents a hydrogen atom, a fluorine atom, a methyl group, or a trifluoromethyl group; L³ represents a single bond, —COO—, or —CONH—; each of R¹⁷, R¹⁸, and R¹⁹ represents a hydrogen atom, a C1 to C20 substituted or unsubstituted monovalent hydrocarbon group, or a C1 to C20 substituted or unsubstituted monovalent oxyhydrocarbon group; R³⁵ represents a monovalent substituent; and g1 is an integer of 0 to 4; and

in formula (iii-3), R³1 represents a hydrogen atom, a fluorine atom, a methyl group, or a trifluoromethyl group; L⁴ represents a single bond, —COO—, or —CONH—; each of R³², R³³, and R³⁴ independently represents a hydrogen atom, a C1 to C20 substituted or unsubstituted monovalent hydrocarbon group, or a C1 to C20 substituted or unsubstituted monovalent oxyhydrocarbon group; R³³ and R³⁴ independently represents a hydrogen atom, a C1 to C20 substituted or unsubstituted monovalent hydrocarbon group, or a C1 to C20 substituted or unsubstituted monovalent oxyhydrocarbon group, or R³³ and R³⁴ taken together represent a C3 to C20 alicyclic hydrocarbon structure together with the carbon atom to which R³³ and R³⁴ bond; R³⁶ represents a monovalent substituent; and g2 is an integer of 0 to 4.)

In the aforementioned formula (iii-1), from the viewpoint of copolymerizability of the monomer forming the structural unit (III-1), R¹² is preferably a hydrogen atom or a methyl group, more preferably a methyl group. In the aforementioned formula (iii-2), from the viewpoint of copolymerizability of the monomer forming the structural unit (III-2), R¹⁶ is preferably a hydrogen atom. Similarly, R³¹ in the aforementioned formula (iii-3) is preferably a hydrogen atom.

Examples of the C1 to C20 monovalent hydrocarbon group represented by any of R¹³ to R¹⁵, R¹⁷ to R¹⁹, and R³² to R³⁴ include a C1 to C20 monovalent chain hydrocarbon group, a C3 to C20 monovalent alicyclic hydrocarbon group, and a C6 to C20 monovalent aromatic hydrocarbon group. Specific examples thereof include the same groups exemplified as the monovalent hydrocarbon group represented by R⁴ to R⁶ in the aforementioned formula (G-1).

Specific examples of the C3 to C20 alicyclic hydrocarbon structure formed by combining R¹⁴ and R¹⁵ together with a carbon atom to which R¹⁴ and R¹⁵ bond, and the C3 to C20 alicyclic hydrocarbon structure formed by combining R³³ and R³⁴ together with a carbon atom to which R³³ and R³⁴ bond include the same structures as exemplified in relation to R⁵ and R⁶ in the aforementioned formula (C-1)

Examples of the C1 to C20 monovalent oxyhydrocarbon group represented by any of R¹⁷ to R¹⁹, and R³² to R³⁴ include groups formed by incorporating an oxygen atom into the chemical bond side end of any of the C1 to C20 monovalent hydrocarbon groups exemplified as those represented by the aforementioned R¹¹ to R¹⁵, R¹⁷ to R¹⁹, and R³² to R³⁴. Among them, the monovalent oxyhydrocarbon group represented by any of R¹⁷ to R¹⁹ and R³² to R³⁴ is preferably an alkoxy group, a cycloalkoxy group, or a cycloalkylalkoxy group.

When the group represented by any of R¹³ to R¹⁹ and R³² to R³⁴ has a substituent, examples of the substituent include a halogen atom, a hydroxy group, and a C1 to C3 alkoxy group. In the case where R¹⁴ and R¹¹ taken together represent a C3 to C20 alicyclic hydrocarbon structure together with the carbon atom to which R¹⁴ and R¹⁵ bond, or R³³ and R³⁴ taken together represent a C3 to C20 alicyclic hydrocarbon structure together with the carbon atom to which R³³ and R³⁴ bond, any of the above-exemplified substituents or an alkyl group may be bound to a ring.

Examples of the monovalent substituent represented by R³⁵ or R³, include a C1 to C3 alkyl group, a C1 to C3 alkoxy group, a hydroxy group, and a halogen atom (e.g., a fluorine atom, a chlorine atom, a bromine atom, or an iodine atom). Each of g1 and g2 is preferably 0 to 2, more preferably 0 or 1.

Specific examples of the structural unit (III-1) include those represented by the following formulas.

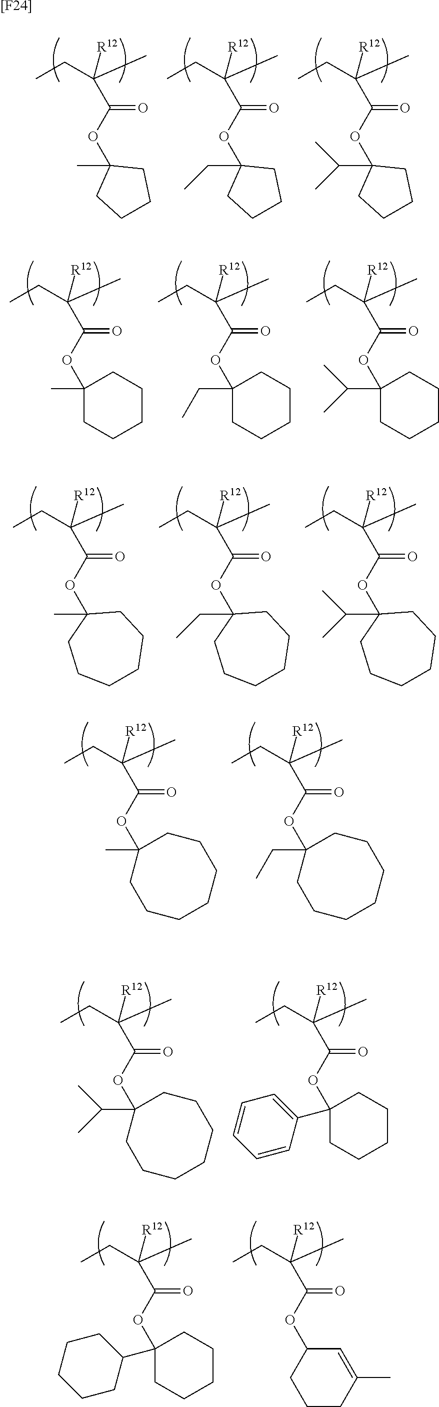

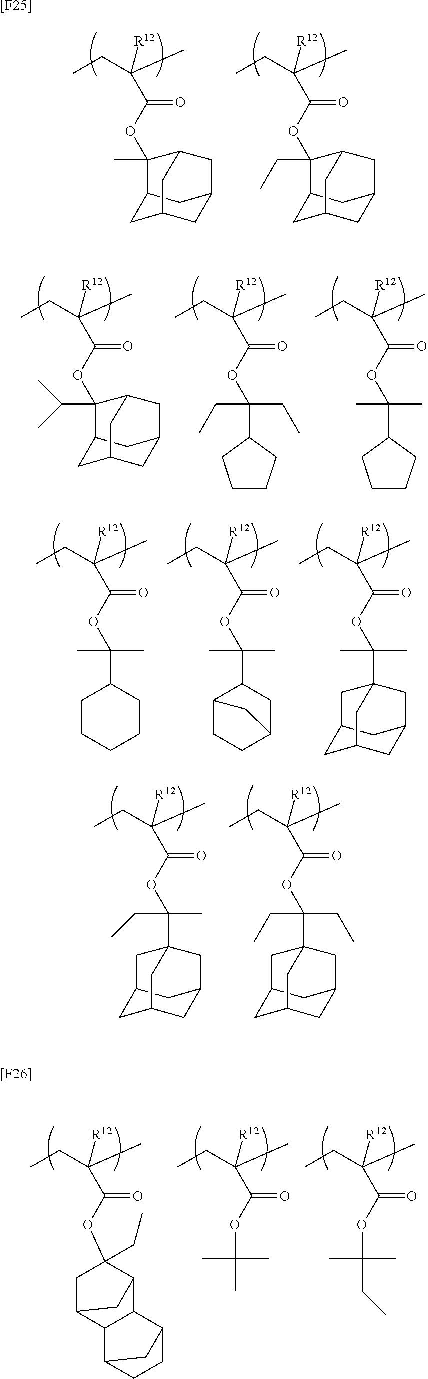

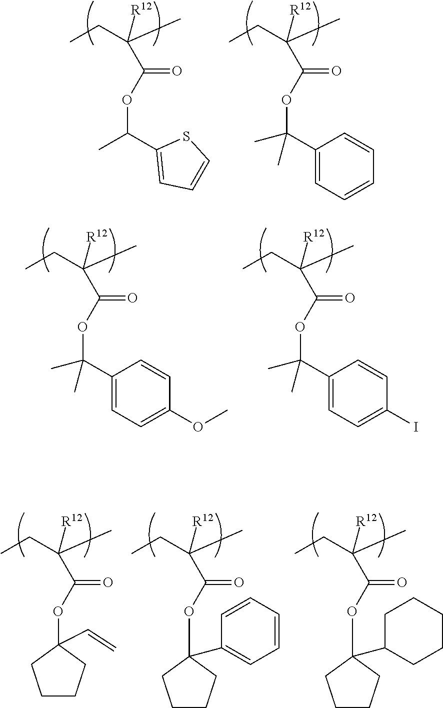

(In the formulas, R¹² represents a hydrogen atom, a fluorine atom, a methyl group, or a trifluoromethyl group.)

Specific examples of the structural unit (III-2) include those represented by the following formulas.

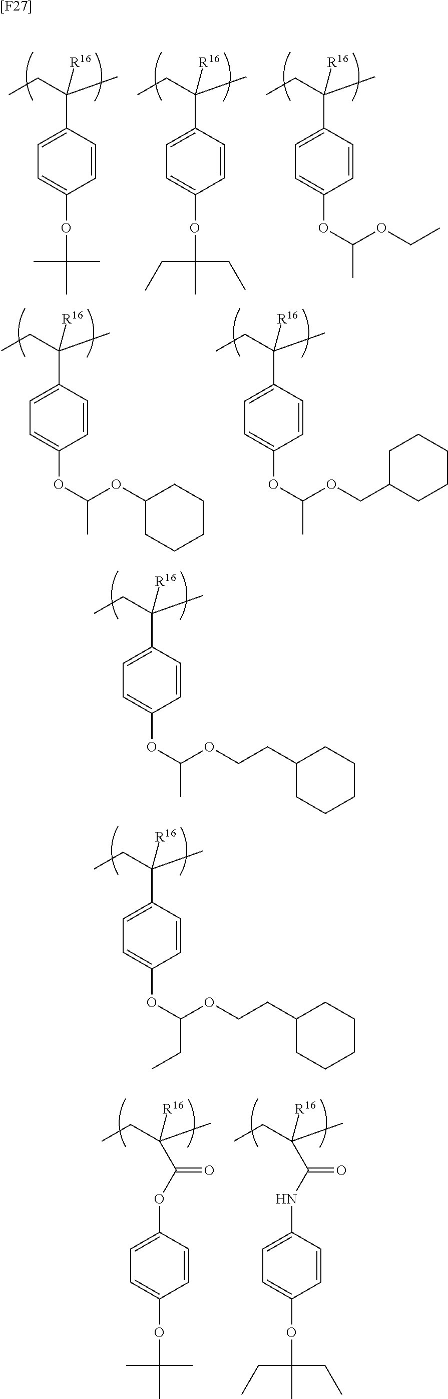

(In the formulas, R⁶ represents a hydrogen atom, a fluorine atom, a methyl group, or a trifluoromethyl group.)

Specific examples of the structural unit (III-3) include those represented by the following formulas.

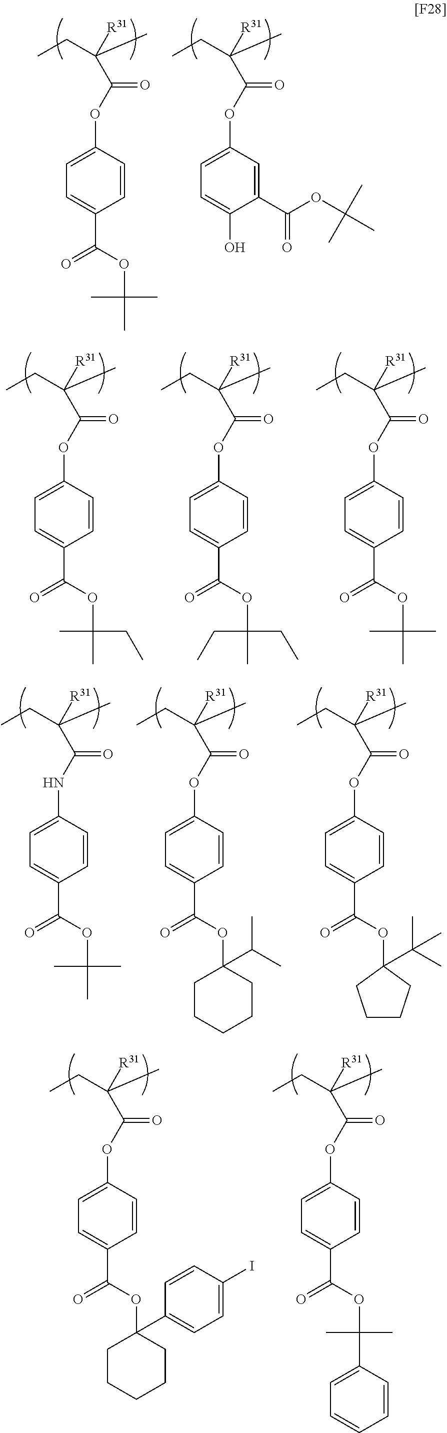

(In the formulas, R³¹ represents a hydrogen atom, a fluorine atom, a methyl group, or a trifluoromethyl group.)

When the polymer [A] include the third structural unit, the relative amount of the third structural unit in all the structural units forming the polymer [A] is preferably 20 mol % or more, more preferably 25 mol % or more, still more preferably 30 mol % or more. Also, the relative amount of the third structural unit in all the structural units forming the polymer [A] is preferably 80 mol % or less, more preferably 75 mol % or less, still more preferably 70 mol % or less. Adjusting the third structural unit content to satisfy the aforementioned conditions is preferred, since a greater difference in dissolution rate with respect to a developer is provided between the light-exposed part and the light-unexposed part, to thereby provide a more suitable pattern shape of the resist film.

Notably, the polymer [A] may include a structural unit in which an acid-releasable group and a hydroxy group are bound to the same aromatic ring or different aromatic rings. As used herein, the structural unit in which an acid-releasable group and a hydroxy group are bound to the same aromatic ring or different aromatic rings is categorized into the third structural unit.

### Fourth Structural Unit

The polymer [A] may further include a structural unit having a lactone structure, a cyclic carbonate structure, a sultone structure, or a cyclic structure of a combination of two or more members thereof (except for the cases corresponding to the first to third structural units) (hereinafter referred to as a “fourth structural unit”) Incorporation of the fourth structural unit into the polymer [A] is preferred, since solubility of the composition in a developer can be controlled, whereby lithographic characteristics of the present composition can be further enhanced. Also, the presence of the fourth structural unit in the polymer [A] can improve close adhesion between a substrate and a resist film formed from the present composition.

Specific examples of the fourth structural unit include those represented by the following formulas.

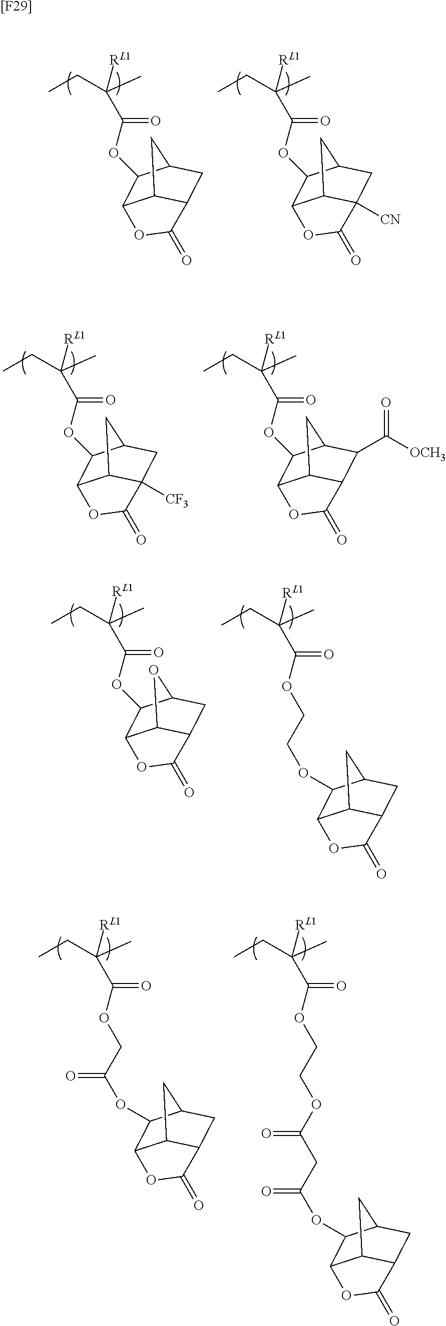

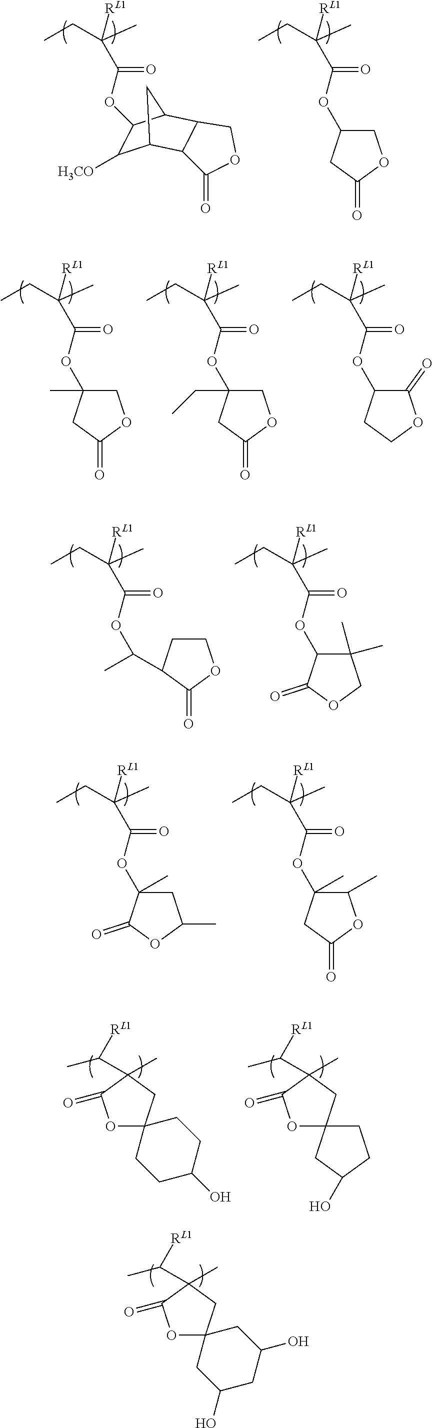

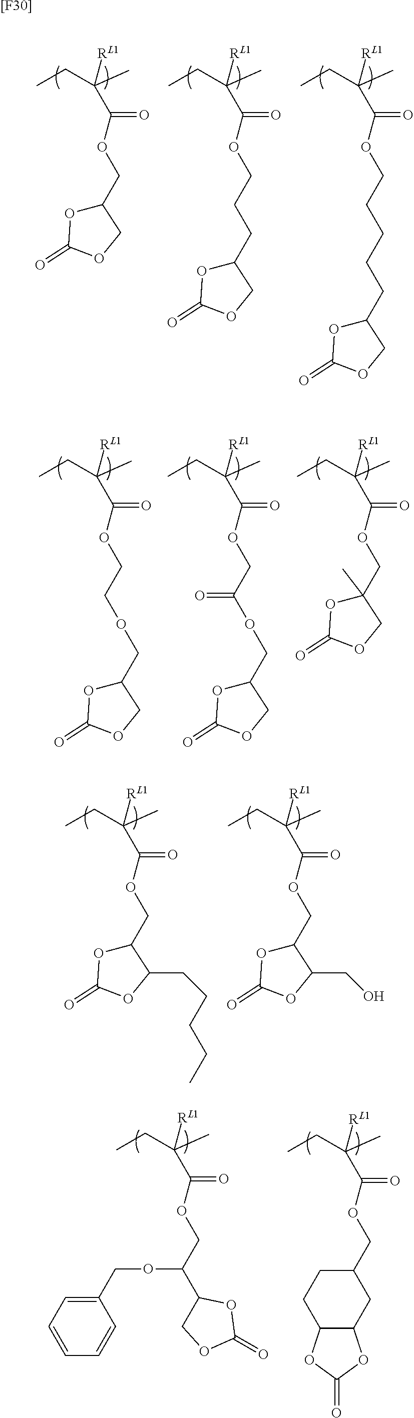

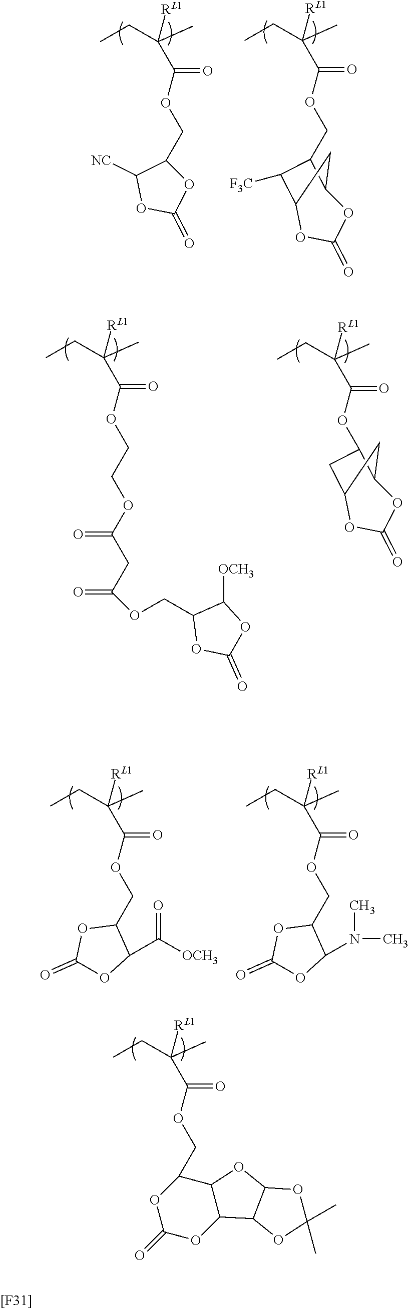

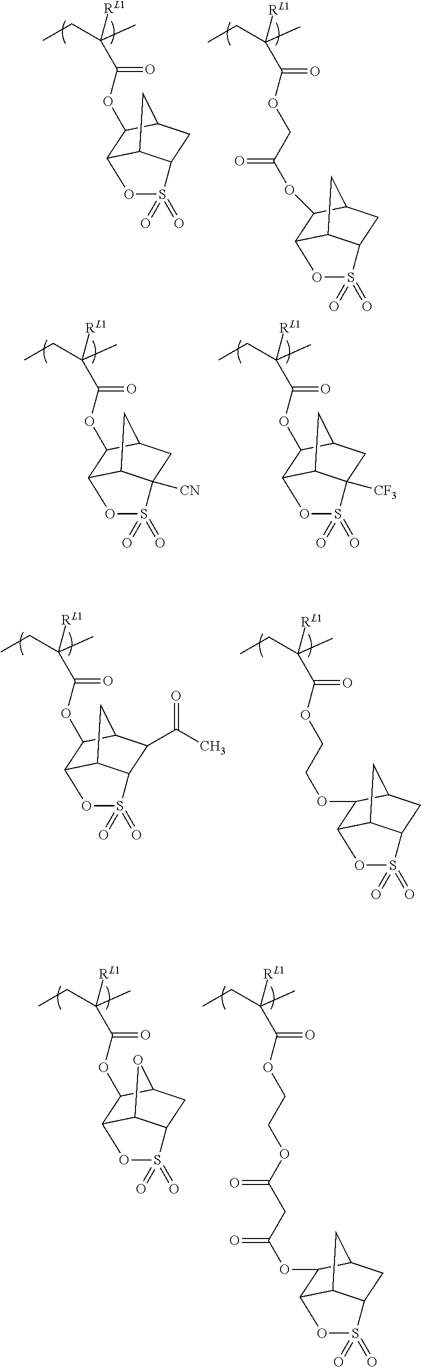

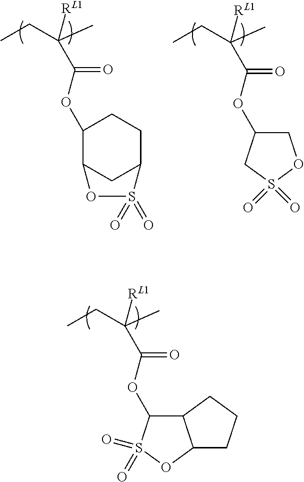

(In the formulas, R^{L1} represents a hydrogen atom, a fluorine atom, a methyl group, or a trifluoromethyl group.)

When the polymer [A] include the fourth structural unit, the relative amount of the fourth structural unit in all the structural units forming the polymer [A] is preferably 1 mol % or more, more preferably 3 mol % or more, still more preferably 5 mol % or more. Also, the relative amount of the fourth structural unit in all the structural units forming the polymer [A] is preferably 50 mol % or less, more preferably 30 mol % or less, still more preferably 15 mol % or less. Adjusting the fourth structural unit content to satisfy the aforementioned conditions is preferred, since lithographic characteristics of the present composition and close adhesion between a substrate and a resist film formed from the present composition can be improved.

### Fifth Structural Unit

The polymer [A] may further include a structural unit having an alcoholic hydroxy group (except for the cases corresponding to the first to fourth units)) (hereinafter referred to as a “fifth structural unit”) As used herein, the term “alcoholic hydroxy group” refers to a group having a structure in which a hydroxy group is directly bonded to an aliphatic hydrocarbon group. The aliphatic hydrocarbon group may be a chain hydrocarbon group or an alicyclic hydrocarbon group. Incorporation of the fifth structural unit into the polymer [A] is preferred, since the solubility of the component(s) in a developer can be improved, whereby lithographic characteristics can be further improved.

The fifth structural unit is preferably derived from an unsaturated monomer having an alcoholic hydroxy group. No particular limitation is imposed on the unsaturated monomer, and examples include 3-hydroxyadamantan-1-yl (meth)acrylate and 2-hydroxyethyl (meth)acrylate.

When the polymer [A] include the fifth structural unit, the relative amount of the fifth structural unit in all the structural units forming the polymer [A] is preferably 1 mol % or more, more preferably 3 mol % or more. Also, the relative amount of the fifth structural unit in all the structural units forming the polymer [A] is preferably 30 mol % or less, more preferably 20 mol % or less.

In addition to the aforementioned units, additional structural units may be acceptable. Examples include a structural unit having a cyano group, a nitro group, or a sulfonamide group (specifically, a structural unit derived from 2-cyanomethyladamantan-2-yl (meth)acrylate or the like), and a structural unit having a non-acid-releasable hydrocarbon group (specifically, a structural unit derived from styrene, a structural unit derived from vinylnaphthalene, a structural unit derived from n-pentyl (meth)acrylate, or the like). The relative amounts of these structural units may be appropriately set individually, so long as the effect of the present disclosure are not impaired.

In the present composition, the polymer [A] content with respect to the entire solid content of the present composition is preferably 50 mass % or more, more preferably 70 mass % or more, still more preferably 85 mass % or more. Also, the polymer [A] content with respect to the entire solid content of the present composition is preferably 99 mass % or less, more preferably 98 mass % or less, still more preferably 95 mass % or less. Preferably, the polymer [A] serves as a base resin of the present composition. As used herein, the term “base resin” refers to a polymer component having a content of 50 mass % or more with respect to the entire solid content of the present composition. The present composition may contain one species or two or more species of polymer [A].

The weight average molecular weight (Mw) of polymer [A], which is determined through gel permeation chromatography (GPC) and is reduced to polystyrene, is preferably 1,000 or more, more preferably 2,000 or more, still more preferably 3,000 or more, yet more preferably 4,000 or more. Also, the Mw of polymer [A] is preferably 50,000 or less, more preferably 30,000 or less, still more preferably 20,000 or less, yet more preferably 15,000 or less. Adjusting the Mw of polymer [A] so as to satisfy the above conditions is preferred, since coatability of the present composition can be improved, and development failure can be sufficiently suppressed.

The ratio (Mw/Mn) of Mw to the number average molecular weight (Mn) of the polymer [A], which is determined through gel permeation chromatography (GPC) and is reduced to polystyrene, is preferably 5.0 or less, more preferably 3.0 or less, still more preferably 2.0 or less, yet more preferably 1.8 or less. Also, the Mw/Mn of the polymer [A] is generally 1 or more, preferably 1.3 or more. The polymer [A] may be synthesized through, for example, polymerization of monomers for providing corresponding structural units in an appropriate solvent in the presence of a known radical polymerization initiator or the like.

### <a Monomer for Providing First Structural Unit and Synthesis of the Monomer>

A monomer that can provide the first structure (hereinafter may also be referred to as a “compound (M)”) is represented by the following formula (2)

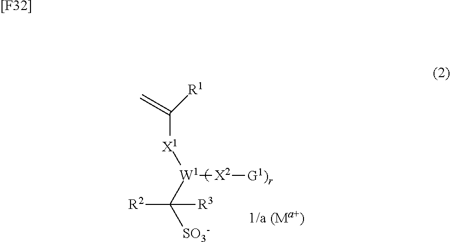

(In formula (2), R¹ represents a hydrogen atom, a fluorine atom, a methyl group, or a trifluoromethyl group; X¹ represents a single bond, *¹—COO—, *¹—CONH—, or a divalent aromatic cyclic group; “*¹” represents a chemical bond with a carbon atom to which R¹ is bound; W¹ represents a (r+2)-valent organic group; X² represents *³—COO— or —O—; “*³” represents a chemical bond with W¹; G¹ represents an acid-releasable group; r is 1 or 2; when r is 2, two GIs in the formula are identical to or different from each other; each of R² and R³ independently represents a fluorine atom or a fluoroalkyl group; M^{a+} represents an a-valent cation; and a is 1 or 2.)

±n the aforementioned formula (2), specific examples and preferred members of R¹, X¹, W¹, X², G¹, r, R², R³, and M^{a+} are the same as those described in relation to R¹, X¹, W¹, X², G¹, r, R², R³, and M^{a+} in the aforementioned formula (1)

The compound (M) can be synthesized through customary methods of organic chemistry in appropriate combinations. Examples of the synthesis method include a method in which an aldehyde compound or a ketone compound having a partial structure represented by “H₂C═C(R¹)—X¹—W¹(X²-G¹)ᵣ) -” is reacted with a diol compound having a partial structure represented by “—C(R²)(R³)—SO₃—” in an appropriate organic solvent optionally in the presence of a catalyst; a method in which a carboxylic acid having a partial structure represented by “H₂C═C(R¹)—X¹—W¹(-(X²-G¹)ᵣ) -” is reacted with a hydroxy group-containing compound having a partial structure represented by “—C(R²)(R³)—SO₃—” in an appropriate organic solvent optionally in the presence of a catalyst; a method in which a hydroxy group-containing compound having a partial structure represented by “H₂C═C(R¹)—X¹—W¹(-(X¹-G¹)ᵣ)-” is reacted with a carboxylic acid having a partial structure represented by “¹—C(R²)(R³)—SO₃—” in an appropriate organic solvent optionally in the presence of a catalyst; and a method in which a halide compound having a partial structure represented by “—W¹(—(X²-G¹)ᵣ)-” is reacted with a compound having a structure represented by “—C(R²)(R³)—SO₃—” in an appropriate organic solvent optionally in the presence of a catalyst, to thereby bond a partial structure represented by “H₂C═C(R¹)—X¹—” to a reaction site of W¹. However, the method of synthesizing the compound (M) is not limited to the above.

### <[Z] Acid Diffusion Control Agent>

The acid diffusion control agent is a component that can suppress chemical reaction by an acid in the light-unexposed part, with a mechanism of suppressing diffusion of the acid in a resist film, the acid generated in the resist film through light exposure. By incorporating the acid diffusion control agent into the present composition, lithographic characteristics (LWR performance and CDU performance) of the present composition can be further enhanced. Furthermore, variation in line width of a resist pattern, which would otherwise be caused by variation in post-exposure time (i.e., duration of time from light exposure to development) can be suppressed, whereby a radiation-sensitive composition having excellent process stability can be obtained. Examples of the acid diffusion control agent include a nitrogen-containing compound and a light-degradable base.

### (Nitrogen-Containing Compound)

As the nitrogen-containing compound, a known nitrogen-containing compound employed for forming a resist pattern may be used. Specific examples of the nitrogen-containing compound include an amino group-containing compound (e.g., alkylamine, aromatic amine, or polyamine), an amide group-containing compound, a urea compound, a nitrogen-containing heterocyclic compound (e.g., N-(undecan-1-yl carbonyloxyethyl)morpholine), a nitrogen-containing compound having an acid-releasable group (e.g., N-(t-butoxycarbonyl)di-n-octylamine, or N-t-butoxycarbonyl-4-hydroxypiperidine) The nitrogen-containing compound may be used singly or in combination of two or more species.

### (Light-Degradable Base)

As the light-degradable base, preferably used is a compound which generates an acid having an acidity lower than that of the acid generated by the polymer [A] upon exposure to light (hereinafter may also be referred to as a “compound (z)”). Meanwhile, the degree of acidity can be evaluated on the basis of acid dissociation constant (pKa). The acid dissociation constant (pKa) of the acid generated by the light-degradable base is generally −3 or higher, preferably −1 to 7, more preferably 0 to 5. Under general conditions, the acid generated by the light-degradable base is a weak acid which does not evoke release of an acid-releasable group. As used herein, the term “general conditions” refers to conducting post-exposure bake (PEB) at 110° C. for 60 seconds.

As the compound (Z), an onium salt compound formed of a cation (more specifically, a radiation-sensitive onium cation) and an organic anion is preferably used. From the viewpoint of enhancing lithographic characteristics of the present composition, the compound (Z) is preferably, among others, an onium salt that can generate carboxylic acid, sulfonic acid, or sulfonamide through light exposure. Also, from the viewpoint of successfully forming a resist film exhibiting higher LWR performance, an onium salt having a sulfonium cation structure or an iodonium cation structure is preferably used as the compound (Z) The compound (Z) may be used singly or in combination of two or more species.

Specific examples of the compound (z) include the following compound (z1) and compound (z2):
Compound (z1): an onium salt formed of an iodine atom-containing organic anion and a cation; and
Compound (z2): an onium salt formed of a fluorine atom-containing cation and an organic anion.

The compounds will next be described in detail.

### Compound (z1)

No particular limitation is imposed on the structure of the organic anion included in the compound (z1) (hereinafter may also be referred to as an “iodine-containing anion”), so long as the anion contains one or more iodine atoms. Examples of the iodine-containing anion include a sulfonate anion structure, an imide anion structure, a methyl anion structure, and a carboxylate anion structure. Among them, the iodine-containing anion preferably includes a sulfonate anion structure or a carboxylate anion structure, more preferably a carboxylate anion structure.

From the viewpoints of achieving high sensitivity of the present composition and securing a wide process margin during pattern formation, the number of iodine atoms belonging to the iodine-containing anion is preferably 2 or greater, more preferably 3 or greater. From the viewpoints of attaining the effect of widening the process margin during pattern formation and ease of synthesizing the compound (z1) in a well-balanced manner, the number of iodine atoms belonging to the iodine-containing anion is preferably 10 or smaller, more preferably 8 or smaller.

No particular limitation is imposed on the bonding position of an iodine atom in the iodine-containing anion. From the viewpoint of achieving a high effect of improving the sensitivity of the present composition, the iodine-containing anion preferably has an aromatic ring and a structure in which an iodine atom or iodine atoms are bound to the aromatic ring. More preferably, two or more iodine atoms are bound to the aromatic ring. When the iodine-containing anion has two or more iodine atoms, the two or more iodine atoms may be bound to the same aromatic ring in the iodine-containing anion or different aromatic rings in the anion. The aromatic ring to which an iodine atom or iodine atoms are bound is preferably a benzene ring or a naphthalene ring, more preferably a benzene ring.

Specific examples of the iodine-containing anion include anions represented by the following formulas (z1-1) to (z1-7), respectively.

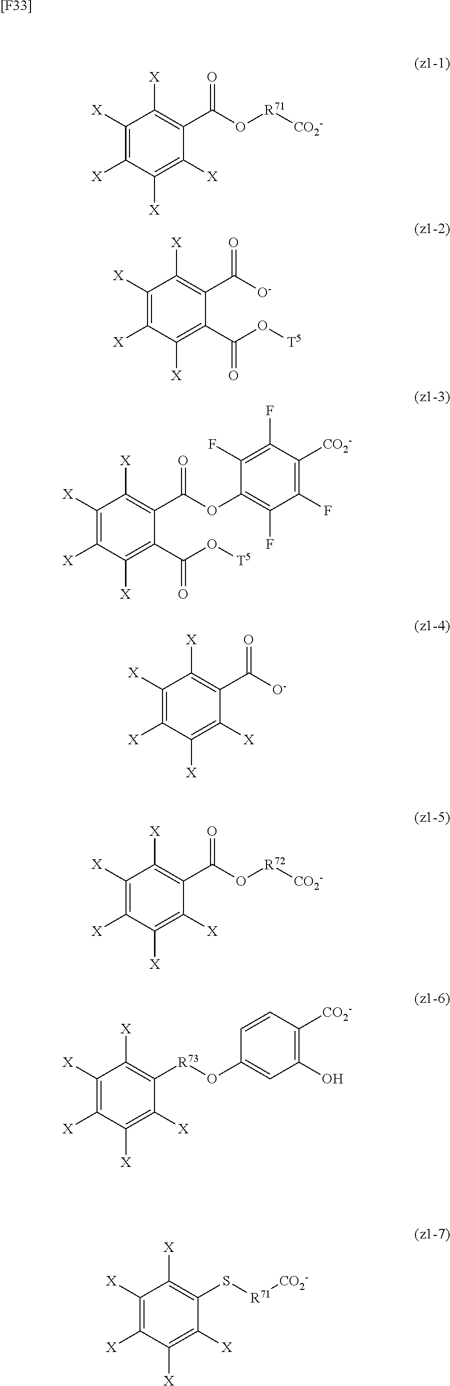

(In formulas (z1-1) to (z1-7), X independently represents a hydrogen atom, a halogen atom, a hydroxy group, a C1 to C3 alkyl group, an amnio group, or an amino group protected by an acid-releasable group; one or more of the plurality of Xs in the respective formula represent iodine atoms; R⁷¹ represents a C1 to C6 alkanediyl group or a C1 to C6 fluoroalkanediyl group; R⁷² represents a fluorinated divalent cyclic group; R⁷³ represents a C1 to C6 alkanediyl group; and T⁵ represents an alkyl group or a cyclo alkyl group.)

In the aforementioned formulas (z1-1) to (z1-7), the C1 to C6 fluoroalkanediyl group represented by R⁷¹ is preferably C1 to C4. Specific examples thereof include —CF₂—, —CF₂—CF₂—, —CH(CF₃)—CF₂—, —CH₂—CF₂—, —CF₂—CH₂—, —C(CF₃)₂—CH₂—, and —CH₂—C(CF₃)₂—. The C1 to C6 alkanediyl group represented by R⁷¹ is preferably C1 to C3, more preferably a methylene group or an ethylene group.

Examples of the fluorinated divalent cyclic group represented by R⁷² include a C3 to C20 monovalent alicyclic hydrocarbon group or a group formed by substituting one or more hydrogen atoms by a fluorine atom in the C6 to C20 monovalent aromatic hydrocarbon group. Specific examples of the alicyclic hydrocarbon group and the aromatic hydrocarbon group include the same groups exemplified as the C1 to C20 monovalent hydrocarbon group represented by any of R⁴ to R⁶ in the aforementioned formula (G-1)

Specific examples of the iodine-containing anion include organic anions represented by the following formulas. However, the structure of the iodine-containing anion is not limited to the following structures.

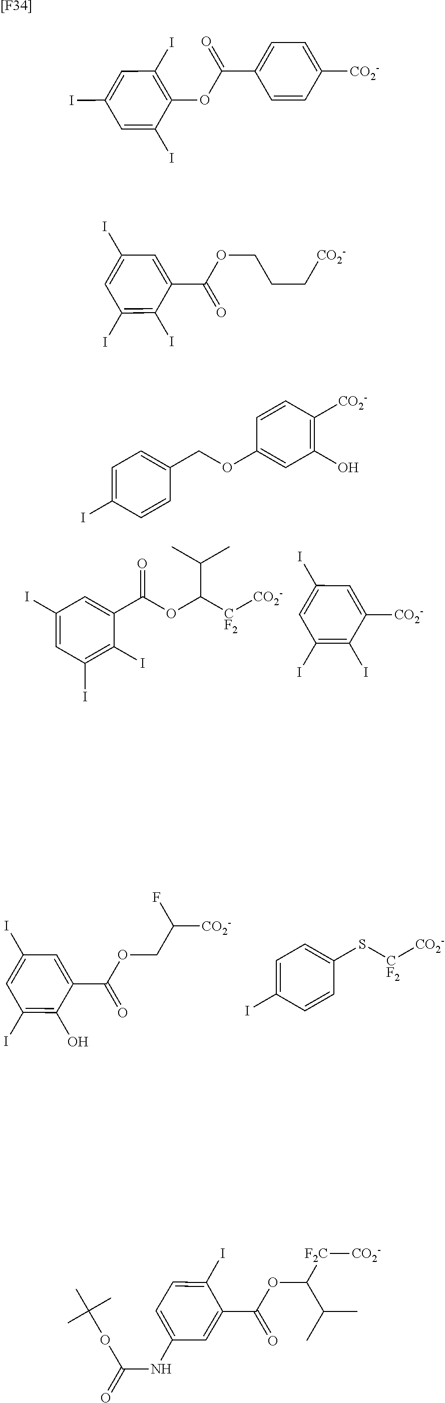

The cation included in the compound (z1) preferably has a sulfonium cation structure or an iodonium cation structure, more preferably a triarylsufonium cation structure or a diaryliodonium cation structure. Specific examples of the cation included in the compound (z1) include the same cations exemplified as the cation (M^{a+}) included in the first structural unit. The cation included in the compound (z1) may contain a fluorine atom.

Specific examples of the compound (z1) include an onium salt which is formed through any combination of an iodine-containing anion and a cation, which are exemplified above. Further specific examples thereof include an onium salt formed of a cation represented by any of the aforementioned formulas (7) to (9) and an organic anion represented by any of the aforementioned formulas (z1-1) to (z1-7).

### Compound (z2)

No particular limitation is imposed on the structure of the cation of the compound (z2) (hereinafter may also be referred to as a “fluorine-containing cation”), so long as the cation has one or more fluorine atoms. From the viewpoint of sensitivity, the fluorine-containing cation preferably includes at least one group selected from the group consisting of a fluoroalkyl group and a fluoro group (except for the fluoro group of the fluoroalkyl group) (hereinafter may also be referred to as a “group Rf¹”).

When the fluorine-containing cation includes a fluoroalkyl group, the fluoroalkyl group may be linear-chain or branched. Specific examples of the fluoroalkyl group include the same groups exemplified as the fluoroalkyl group represented by R² or R³ in the aforementioned formula (1)

Among them, from the viewpoint of sensitivity, the group Rf¹ is preferably a fluoro group, a trifluoromethyl group, a 2,2,2-trifluoroethyl group, or a perfluoroethyl group, more preferably a fluoro group or a trifluoromethyl group.

When the fluorine-containing cation includes the group Rf¹, the number of the groups Rf¹s is preferably 2 or greater, more preferably 3 or greater, from the viewpoint of achieving more enhanced sensitivity and LWR performance of the present composition. Also, from the viewpoints of attaining the effect of enhancing the sensitivity and ease of synthesis in a well-balanced manner, the number of the groups Rf¹s is preferably 10 or less, more preferably 8 or less, still more preferably 7 or less, yet more preferably 6 or less.

Notably, when the fluorine-containing cation includes a fluoroalkyl group as the group Rf¹, the number of fluoroalkyl group(s) in the fluorine-containing cation corresponds to the number of the group(s) Rf¹(s) belonging to the fluorine-containing cation. Thus, for example, when the fluorine-containing cation includes two trifluoromethyl groups (—CF₃), the number of the groups Rf¹s belonging in the fluorine atom cation is 2. Also, when the fluorine-containing cation includes one fluoro group (—F) bound to the aromatic ring and two trifluoromethyl groups (—CF₃), the number of the groups Rf's belonging in the fluorine atom cation is 3.

No particular limitation is imposed on the bonding position of the group Rf¹ in the fluorine-containing anion. From the viewpoint of achieving a high effect of improving the sensitivity of the present composition, preferably, one or more groups of Rf¹ are directly bound to the aromatic ring in the fluorine-containing cation, more preferably two or more groups of Rf¹ are directly bound to the aromatic ring in the fluorine-containing cation. Notably, when the fluorine-containing cation has two or more groups Rf¹s, the two or more groups Rf¹ may be bound to the same aromatic ring or the different aromatic rings in the fluorine-containing cation. Among them, preferably, the fluorine-containing cation has one or more aromatic rings bound to a sulfonium cation or an iodonium cation (hereinafter may also be referred to as an “aromatic ring R^{A}”), and the group Rf¹ is directly bound to the aromatic ring R^{A}.

Examples of the aromatic ring RA include a benzene ring, a naphthalene ring, an anthracene ring, and a phenanthrene ring. Of these, the aromatic ring RA is preferably a benzene ring or a naphthalene ring, particularly preferably a benzene ring. In the fluorine-containing cation, the total number of the groups Rf¹s bound to the aromatic ring R^{A} is preferably 2 or greater, more preferably 3 or greater. From the viewpoints of attaining the effect of enhancing the sensitivity and ease of synthesizing the compound (z2) in a well-balanced manner, the total number of the groups Rf¹s bound to the aromatic ring RA is preferably 10 or less, more preferably 8 or less, still more preferably 7 or less, yet more preferably 6 or less. When the total number of the groups Rf¹s bound to the aromatic ring RA is 2 or greater, each group Rf¹ may be bound to the same aromatic ring or the different aromatic rings in the fluorine-containing cation.

From the viewpoint of sensitivity, the fluorine-containing cation preferably includes a triarylsufonium cation structure or a diaryliodonium cation structure. Specific examples of preferred fluorine-containing cations include a cation in which one or more of R^{1a}, R^{2a}, and R^{3a} in the aforementioned formula (7) are groups Rf¹s, a cation in which one or more of R^{4a} and R^{5a} in the aforementioned formula (8) are groups Rf¹s, and a cation in which R^{6a} in the aforementioned formula (9) is a group Rf¹.

Specific examples of the fluorine-containing cation includes the cations exemplified as the specific examples of M^{a+} in the first structural unit having the group Rf¹. However, the structure of the fluorine-containing cation is not limited to the above structures.

Examples of the organic anion included in the compound (z2) include anions each having a sulfonate anion structure, an imide anion structure, a methyl anion structure, and a carboxylate anion structure. Among them, the organic anion included in the compound (z2) preferably has a sulfonate anion structure or a carboxylate anion structure, more preferably a carboxylate anion structure. Also, the organic anion included in the compound (z2) may have an iodine atom.

Specific examples of the organic anion included in the compound (z2) include organic anions represented by the following formula and organic anions exemplified as the iodine-containing anion. However, the organic anion included in the compound (z2) is not limited to the these structures.

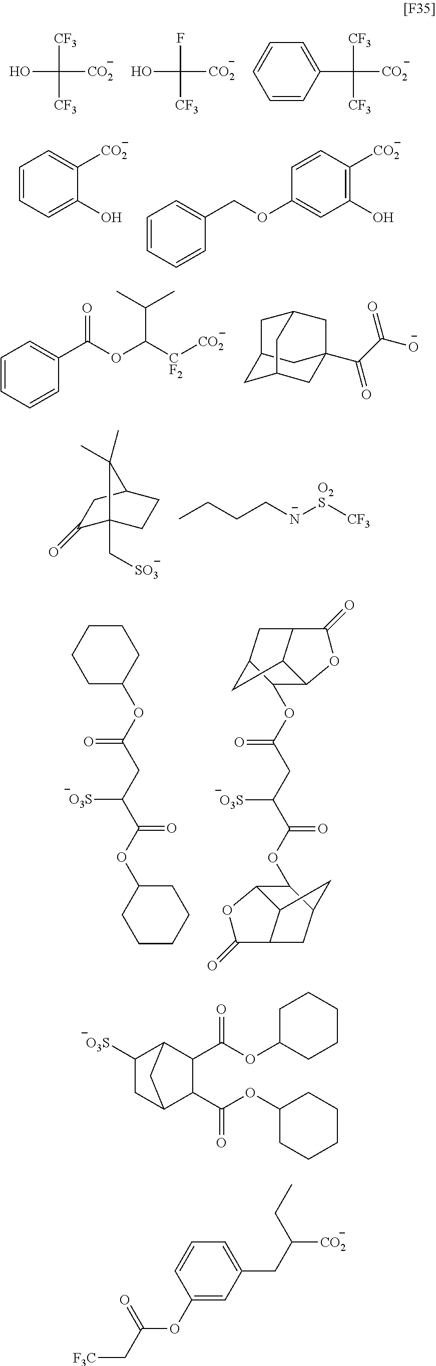

Specific examples of the compound (z2) include an onium salt which is formed through any combination of an fluorine-containing cation and an organic anion, which are exemplified above. Further specific examples thereof include an onium salt formed of an organic anion as exemplified above and a cation in which one or more of R^{1a}, R^{2a}, and R^{3a} in the aforementioned formula (7) are groups Rf¹s, a cation in which one or more of R^{4a} and R^{5a} in the aforementioned formula (8) are groups Rf¹s, and a cation in which R^{6a} in the aforementioned formula (9) is a group Rf¹.

Alternatively, as the light-degradable base, a compound differing from the compound (z1) and the compound (z2) (hereinafter may also be referred to as an “additional light-degradable base”) may be used. Examples of the additional light-degradable base include an onium salt formed of a cation and an anion, wherein the cation is one of those exemplified as cations forming the compound (z1) and the compound (z2) but has no fluorine atom, and the anion is one of those exemplified as anions forming the compound (z1) and the compound (z2) but has no iodine atom, and a compound in which a group having an anion structure is bound to the aryl group of the triarylsulfonium cation structure or a diaryliodonium cation structure (e.g., compounds represented by the following formulas (z3-1) to (z3-3), respectively).

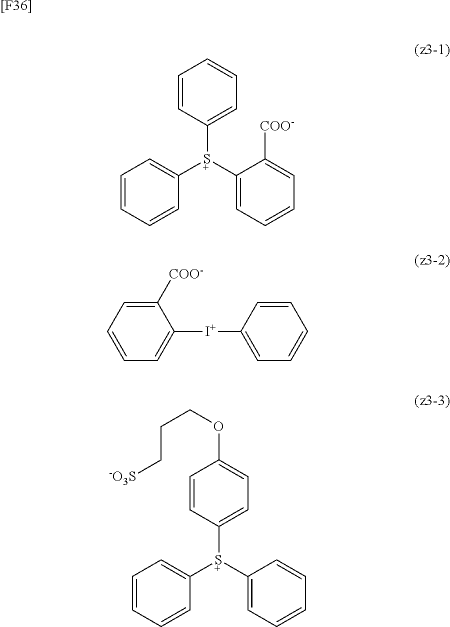

The acid diffusion control agent incorporated into the present composition is preferably a light-degradable base among the above-exemplified compounds, from the viewpoint of improving sensitivity, LWR performance, and process margin during pattern formation in a well-balanced manner, more preferably at least one species selected from the group consisting of the compound (z1) and the compound (z2).

When the present composition contains an acid diffusion control agent, the relative amount of the acid diffusion control agent in the present composition, with respect to the total amount (100 parts by mole) of the amount of the monomer which provides the first structural unit of the polymer [A] contained in the present composition (compound (M)) and the amount of the radiation-sensitive acid-generating agent [B], is preferably adjusted to 1 part by mole or more, more preferably 2 parts by mole or more, still more preferably 5 parts by mole or more. Also, the relative amount of the acid diffusion control agent, with respect to the total amount (100 parts by mole) of the amount of the monomer which provides the first structural unit of the polymer [A] contained in the present composition and the amount of the radiation-sensitive acid-generating agent [B], is preferably adjusted to 150 parts by mole or less, more preferably 140 parts by mole or less, still more preferably 135 parts by mole or less. By adjusting the acid diffusion control agent content to satisfy the above conditions, the LWR performance of the present composition can be further enhanced. The acid diffusion control agent may be used singly or in combination of two or more species.

### <[B] Radiation-Sensitive Acid-Generating Agent>

No particular limitation is imposed on the radiation-sensitive acid-generating agent, and a known radiation-sensitive acid-generating agent may be appropriately used in formation of a resist pattern. An example of the radiation-sensitive acid-generating agent to be incorporated to the present composition is an onium salt formed of a radiation-sensitive onium cation and an organic anion. Typically, the radiation-sensitive acid-generating agent is a compound which evokes release of the acid-releasable group under the aforementioned general conditions, to thereby generate, in the composition, an acid having an acidity higher than that of the acid generated by the light-degradable base (preferably, a strong acid such as a sulfonic acid, an imidic acid, or a methidic acid)

When an onium salt is used as the radiation-sensitive acid-generating agent, the onium cation of the radiation-sensitive acid-generating agent is preferably a sulfonium cation or a iodonium cation. Among them, a triarylsufonium cation or a diaryliodonium cation are preferred. Specific examples thereof include the same cations exemplified as the cation in the aforementioned formula (7) and the cation in in the aforementioned formula (8).

No particular limitation is imposed on the organic anion included in the radiation-sensitive acid-generating agent, so long as it is a compound that can generate an acid upon exposure of the present composition to light. Of these, a sulfonate anion, an imide anion, and a methide anion are preferred.

Specific examples of the organic anion forming the radiation-sensitive acid-generating agent include anions represented by the following formulas. However, the organic anion forming the radiation-sensitive acid-generating agent is not limited to the following structures.

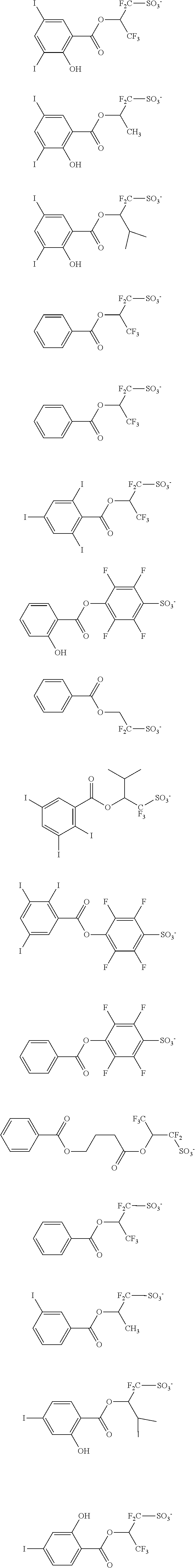

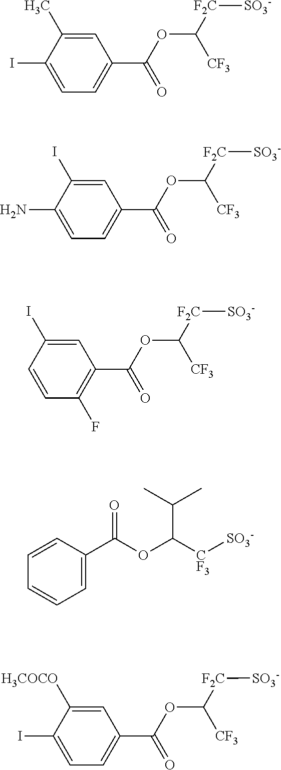

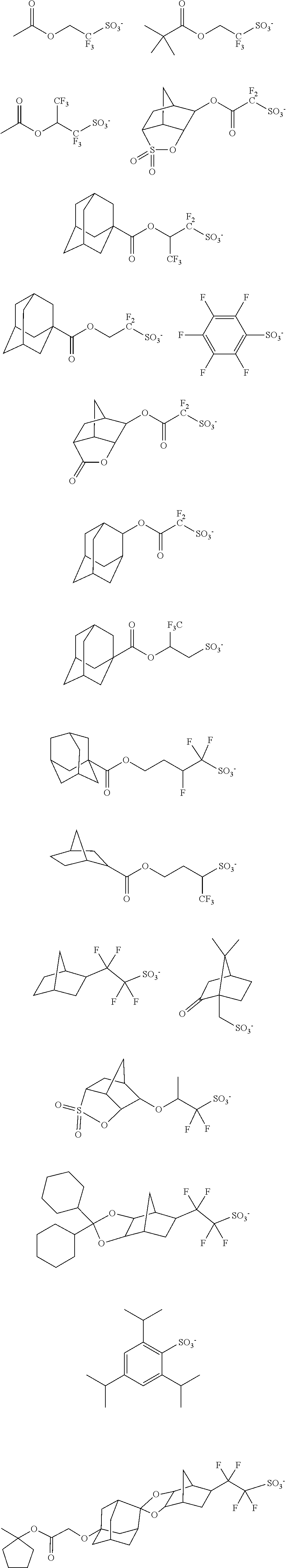

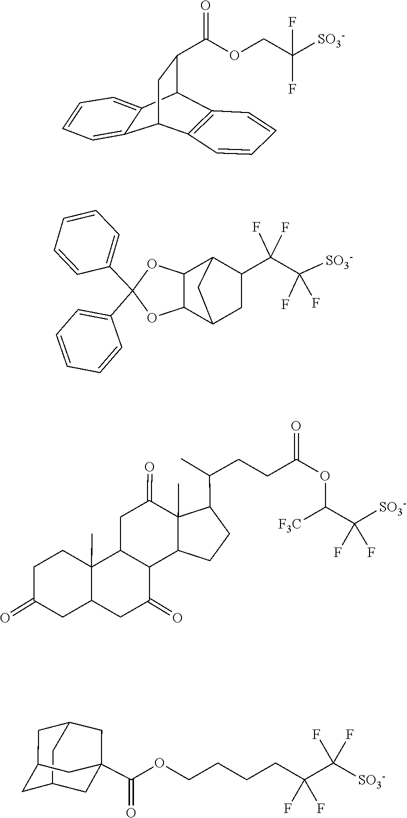

When the radiation-sensitive acid-generating agent is incorporated into the present composition, the relative amount of the radiation-sensitive acid-generating agent, with respect to 100 parts by mass of the polymer [A], is preferably 1 part by mass or more, more preferably 2 parts by mass or more. Also, the relative amount of the radiation-sensitive acid-generating agent, with respect to 100 parts by mass of the polymer [A], is preferably 30 parts by mass or less, more preferably 20 parts by mass or less, still more preferably 15 parts by mass or less. The radiation-sensitive acid-generating agent may be used singly or in combination of two or more species.

### <[D] Solvent>

No particular limitation is imposed on the solvent [D], so long as the solvent can dissolve or disperse the components incorporated into the present composition. Examples of the solvent [D] include an alcohol, an ether, a ketone, an amide, an ester, and a hydrocarbon.

Examples of the alcohol include C1 to C18 aliphatic monoalcohols such as 4-methyl-2-pentanol and n-hexanol; C3 to C18 alicyclic monoalcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl isobutyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amide include cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the ester include monocarboxylic acid ester-type solvents such as n-butyl acetate, ethyl lactate, and methyl 2-hydroxyisobutyrate; polyhydric alcohol carboxylates such as propylene glycol acetate; polyhydric alcohol partial ester carboxylates such as propylene glycol monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Among the above solvents, the solvent [D] preferably includes at least one member selected from the group consisting of the ester and the ketone, more preferably at least one member selected from the group consisting of polyhydric alcohol partial ether carboxylates and cyclic ketones. The solvent [D] may be used singly or in combination of two or more species.

### <[F] High Fluorine-Content Polymer>

The high-fluorine content polymer [F](hereinafter may also be referred to simply as a “polymer [F]”) is a polymer having a fluorine atom content (by mass) greater than that of the polymer [A]. The polymer [F] is incorporated into the present composition as, for example, a water-repellent additive.

No particular limitation is imposed on the fluorine atom content of the polymer [F], so long as it is greater than the fluorine atom content of the polymer [A]. The fluorine atom content of the polymer [F] is preferably 1 mass % or more, more preferably 4 mass % or more, still more preferably 7 mass % or more. Also, the fluorine atom content of the polymer [F] is preferably 60 mass % or less, more preferably 40 mass % or less. The fluorine atom content (mass %) of a polymer can be obtained by determining the structure of the polymer through ¹³C-NMR spectrometry or the like and calculating the content based on the structure determined.

When the present composition contains the polymer [F] the relative amount of the polymer [F] in the present composition, with respect to 100 parts by mass of the polymer [A], is preferably 0.05 parts by mass or more, more preferably 0.1 parts by mass or more, still more preferably 0.5 parts by mass or more. Also, the relative amount of the polymer [F] in the present composition, with respect to 100 parts by mass of the polymer [A], is preferably 10 parts by mass or less, more preferably 5 parts by mass or less, still more preferably 3 parts by mass or less. The polymer [F] may be used singly or in combination of two or more species.

### <Additional and Optional Component>

The present composition may further contain a component which differs from the aforementioned polymer [A], acid diffusion control agent [Z], radiation-sensitive acid-generating agent [B]I, solvent [D], and high fluorine-content polymer [F](hereinafter may also be referred to as an “additional and optional component”). Examples of the additional and optional component include a surfactant, a compound having an alicyclic skeleton (e.g., 1-adamantanecarboxylic acid, 2-adamantanone, or t-butyl deoxycholate), a sensitizer, a localization accelerator, and a nitrogen-containing compound. So long as the effect of the present disclosure are not impaired, the additional and optional component content of the present composition may be appropriately modified depending on the components.

### <Method of Producing Radiation-Sensitive Composition>

The present composition may be produced through, for example, the following procedure: mixing the polymer [A] with optional components such as the solvent [D] at a desired ratio and filtering the resultant mixture preferably by means of a filter (e.g., a filter having a pore size of about 0.2 μm) or the like. The solid content of the present composition is preferably 0.1 mass % or more, more preferably 0.5 mass % or more, still more preferably 1 mass % or more. Also, the solid content of the present composition is preferably 50 mass % or less, more preferably 20 mass % or less, still more preferably 5 mass % or less. Adjusting the solid content of the present composition to satisfy the above conditions is preferred, since coatability of the composition can be enhanced, to thereby obtain a resist pattern having a suitable shape.

The thus-obtained present composition may also be used as a composition for forming a positive pattern, which is employed for pattern formation by use of an alkaline developer. Alternatively, the present composition may be used as a composition for forming a negative pattern by use of a developer containing organic solvent.

### <Method of Forming Resist Pattern>

The resist pattern formation method of the present disclosure includes a step of applying the present composition on one surface of a substrate (hereinafter may also be referred to as a “application step”), a step of exposing to light a resist film obtained in the application step (hereinafter may also be referred to as a “light-exposure step”), and a step of developing the light-exposed resist film (hereinafter may also be referred to as a “development step”). Examples of the pattern obtained through the resist pattern formation method of the present disclosure include a line-and-space pattern and a hole pattern. Since a resist film is formed by use of the present composition in the resist pattern formation method of the present disclosure, a resist pattern which exhibits excellent sensitivity and lithographic characteristics and which has few development failure can be formed. The steps will next be described in detail.

### [Application Step]

In the application step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum. Alternatively, an organic or inorganic anti-reflection film (see, for example, Japanese Patent Application Laid-Open (kokai) No. 1984-93448) may be formed on a substrate to be used. Examples of the method of applying the present composition include spin coating, flow casting, and roller coating. After application, the applied composition may be subjected to soft baking (SE, also called pre-baking) so as to evaporate the solvent remaining in the coating film. The temperature of SB is preferably 60° C. or higher, more preferably 80° C. or higher, and preferably 140° C. or lower, more preferably 120° C. or lower. The time of SB is preferably 5 seconds or longer, more preferably 10 seconds or longer, and preferably 600 seconds or shorter, more preferably 300 seconds or shorter. The average thickness of the formed resist film is preferably 10 to 1,000 nm, more preferably 20 to 500 nm.

### [Light-Exposure Step]

In the light-exposure step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a UV ray, a far-JU ray, an extreme UV (EUV) ray, an X-ray, and a γ-ray; and charged particle rays such as an electron beam and an α-ray. Among them, the radiation applied to the resist film formed from the present composition is preferably a far-TV ray, an EUT ray, or an electron beam, more preferably ArF excimer laser light (wavelength: 193 nm), KrF excimer laser light (wavelength: 248 nm), an ET ray, or an electron beam, still more preferably ArF excimer laser light, an ETN ray, or an electron beam, yet more preferably an ETV ray or an electron beam, particularly preferably an EUV ray.

After completion of the above light exposure, post exposure baking (PEB) is preferably performed, whereby release of the acid-releasable group, caused by the acid generated by a compound (i.e., the polymer [A] or the radiation-sensitive acid-generating agent [B]) that can generate acid through exposure to light in the light-exposed part of the resist film, is accelerated. Through PEE, the difference in dissolution performance with respect to a developer between the exposed part and the unexposed part can be increased. The temperature at PEE is preferably 50° C. or higher, more preferably 80° C. or higher, and preferably 180° C. or lower, more preferably 130° C. or lower. The time of PEE is preferably 5 seconds or longer, more preferably 10 seconds or longer, and preferably 600 seconds or shorter, more preferably 300 seconds or shorter.

### [Development Step]

In this step, the resist film which has been exposed to light in the above step is developed, whereby a resist pattern of interest can be formed. Generally, after development, the developed film is washed with a rinse liquid (e.g., water or alcohol) and then dried. The development method employed in the development step may be development with alkali or with organic solvent.

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, 1,5-diazabicyclo[4.3.0]-5-nonene, and the like is dissolved. Among such alkaline solutions, an aqueous TMAH solution is preferred, with a 2.38 mass % aqueous TMAH solution being more preferred.

In the case of development with an organic solvent, examples of the developer include one or more members of organic solvents such as hydrocarbnons, ethers, esters, ketones, and alcohols, and a solvent containing any of the organic solvents. Examples of the organic solvent used as a developer include the solvents listed as the solvent [E] in relation to the description of the present composition. Among them, an ester and a ketone are preferred. The ester is preferably an acetate ester, more preferably n-butyl acetate. The ketone is preferably a chain ketone, more preferably 2-heptanone. The organic solvent content of the developer is preferably 80 mass % or more, more preferably 90 mass % or more, still more preferably 95 mass % or more, particularly preferably 99 mass % or more. Examples of developer components other than organic solvent include water and silicone oil.

Examples of the development method include a dipping method (i.e., dipping a substrate in a bath filled with a developer for a specific time); a paddle method (i.e., putting a developer on a substrate to form a drop by surface tension and allowing the substrate to stand for a specific time); a spray method (i.e., spraying a developer onto a substrate); and a dynamic dispense method (i.e., continuously jetting a developer at a specific speed to a substrate rotating at a specific speed through a developer jetting nozzle with scanning)

### Examples

The present disclosure will next be described in detail by way of examples, which should not be construed as limiting the disclosure to the Examples. Unless otherwise specified, the units “part(s)” and “%” in the Examples are on a mass basis. Physical properties were determined through the following procedures.

### [Weight Average Molecular Weight (Mw) and Number Average Molecular Weight (Mn) of Polymer]

The weight average molecular weight (Mw) and the number average molecular weight (Mn) of a polymer were determined through gel permeation chromatography (GPC) with GPC columns (G2000HXL×2, G3000HXL×1, and G4000HXL×1) (products of Tosoh Corp.) under the following conditions.
Eluent: tetrahydrofuran (product of FUJIFILM Wako Pure Chemical Corporation)
Flow rate: 1.0 mL/min
Sample concentration: 1.0 mass %
Sample injection: 100 μL
Column temperature: 40° C.
Detector: differential refractometer
Standard: monodispersed polystyrene

### Synthesis of Compounds (M)

### [Synthesis Example 1-1] (Synthesis of Monomer (M-15))

Monomer (M-15) was synthesized through the following reaction scheme.

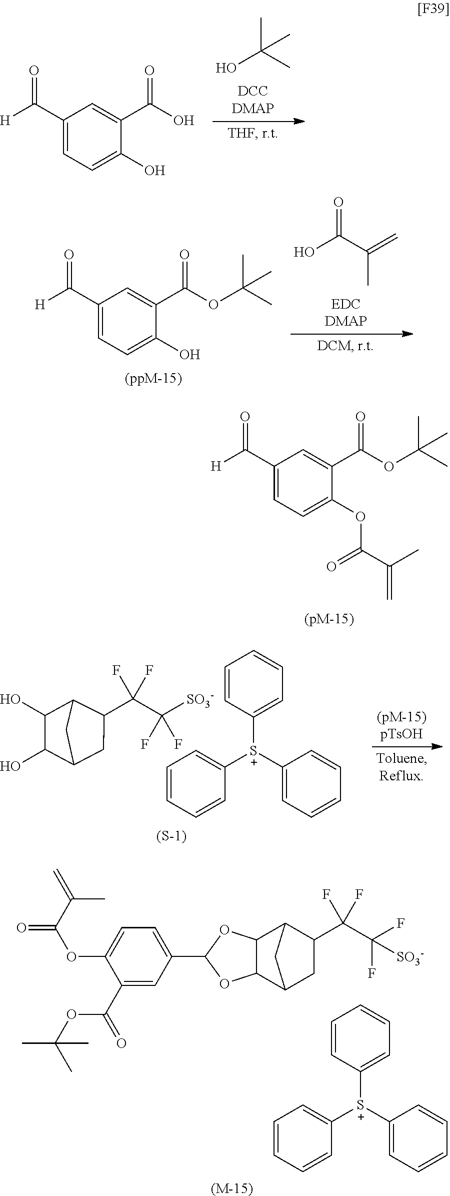

To a reaction vessel, 5-formylsalicylic acid (40 mmol) tert-butanol (200 mmol), and DMAP (4-dimethylaminopyridine) (8 mmol) were added, and the mixture was stirred for 1 hour. DCC (N,N′-dicyclohexylcarbodiimide) (44 mmol) was dissolved in THF (tetrahydrofuran) (80 mL), and the solution was added dropwise to the above mixture. The resultant mixture was stirred at room temperature for 3 hours. The solid was removed through filtration and purified through silica gel chromatography, to thereby yield ppM-15.

To a reaction vessel, ppM-15 (30 mmol), methacrylic acid (39 mmcl), EDC (1-(3-dimethylaminopropyl)-3-ethylcarbocdiim ide) (39 mmol), DMAP (6 mmol), and DCM (dichloromrethane) (40 mL) were added, and the mixture was stirred at room temperature for 2 hours. 2M hydrochloric acid (20 mL) was added thereto, and the organic layer was separated. The organic layer was washed twice with saturated aqueous sodium hydrogen carbonate (40 mL) and once with ultrapure water (40 mL), to thereby yield pM-15.

To a reaction vessel, a compound represented by the aforementioned formula (S-1) (20 mmol), pM-15 (21 mmol), pTsOH (p-toluenesulfonic acid) (4 mmol), and toluene (100 mL) were added. A Dien-Stark tube was attached to the reaction vessel, and the contents of the reaction vessel were stirred for 4 hours under reflux conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-15)

### [Synthesis Example 1-2] (Synthesis of Monomer (M-16))

The procedure of Synthesis Example 1-1 was repeated, except that appropriately selected substrates were used in Synthesis Example 1-1, to thereby synthesize monomer (M-16).

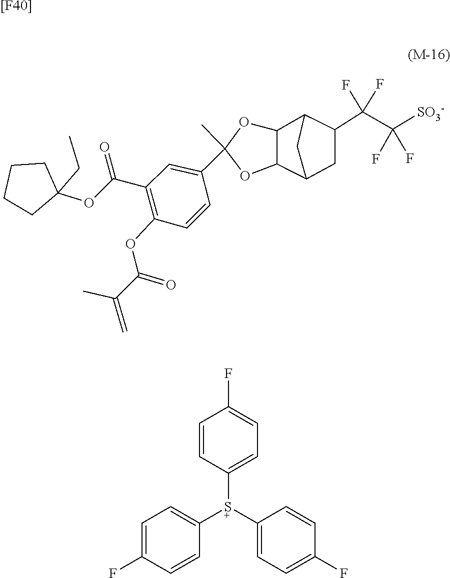

### [Synthesis Example 1-3] (Synthesis of Monomer (M-17))

Monomer (M-17) was synthesized through the following reaction scheme.

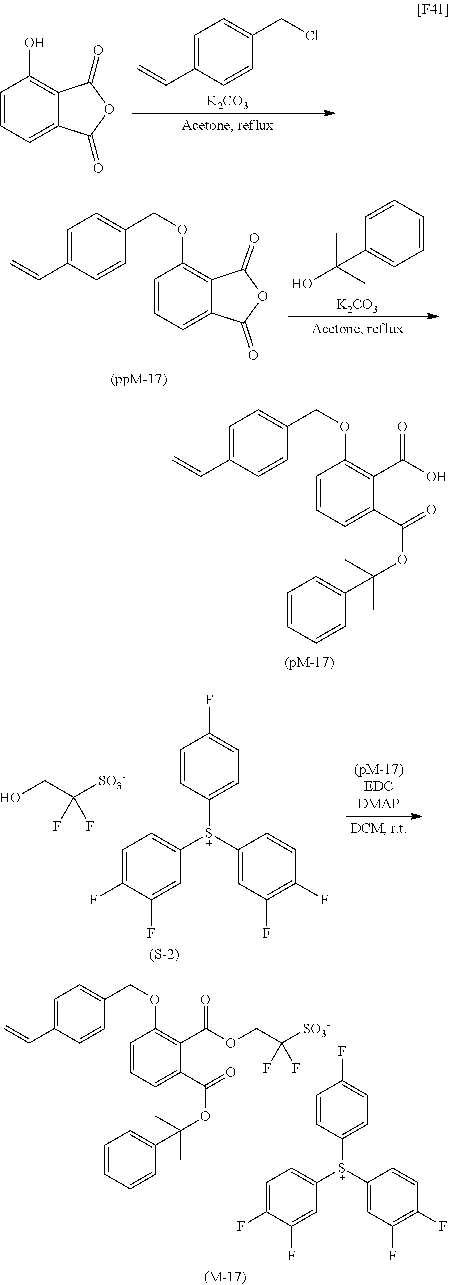

To a reaction vessel, 3-hydroxyphthalic acid anhydrate (30 mmol), potassium carbonate (150 mmol), and acetone (100 mL) were added, and the mixture was stirred for 1 hour. 4-(Choromethyl)styrene (60 mmol) was added dropwise to the mixture, and the resultant mixture was stirred at 55° C. for 5 hours. The mixture was cooled to room temperature, and the precipitated solid was removed through filtration. The filtrate was concentrated and purified through silica gel column chromatography, to thereby yield ppM-17.

To a reaction vessel, 2-phenyl-2-propanol (30 mmol), potassium carbonate (30 mmol), ppM-17 (20 mmol), and acetone (100 mL) were added, and the mixture was stirred under reflux conditions for 5 hours. The resultant mixture was cooled to room temperature, and the precipitated solid was removed through filtration. The filtrate was concentrated and purified through silica gel column chromatography, to thereby yield pM-17.

To a reaction vessel, pM-17 (13 mmol), a compound represented by the aforementioned formula (S-2) (10 mmol), EDC (13 mmol), DMAP (2 mmol), and dichlorormethane (40 mL) were added, and the mixture was stirred for 2 hours. 2M hydrochloric acid (40 mL) was added thereto, and the organic layer was separated. The organic layer was washed twice with saturated aqueous sodium hydrogen carbonate (40 mL) and once with ultrapure water (40 mL), and purified through silica gel column chromatography, to thereby yield monomer (M-17).

### [Synthesis Example 1-4] (Synthesis of Monomer (M-18))

Monomer (M-18) was synthesized through the following reaction scheme.

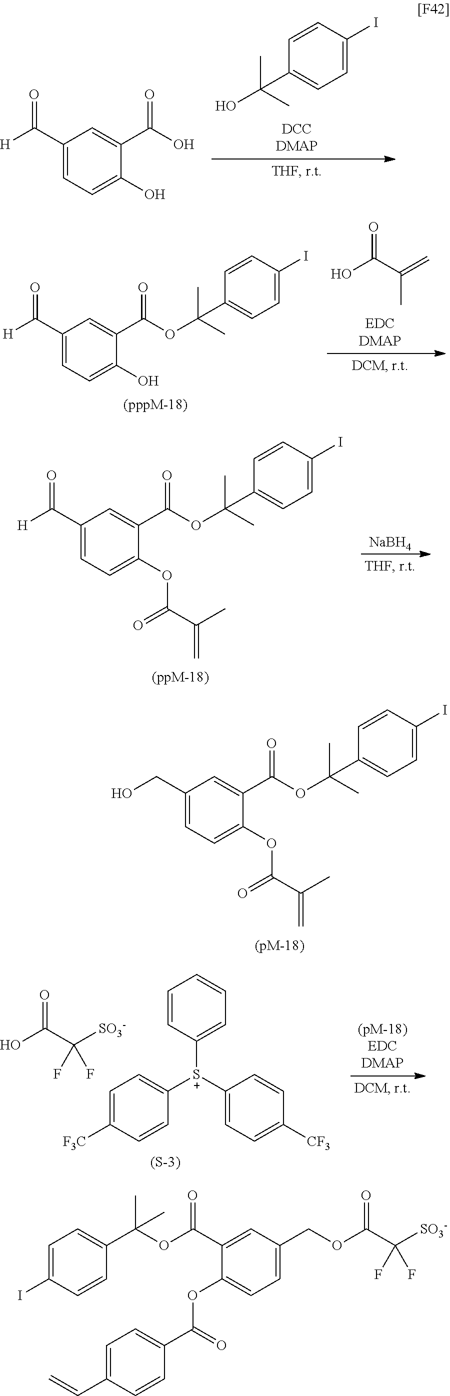

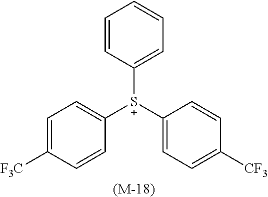

To a reaction vessel, 5-formylsalicylic acid (40 mmol), 2-(4-iodophenyl)-2-propanol (80 mmol), and DMAP (8 mmol) were added, and the mixture was stirred for 1 hour. DCC (44 mmol) was dissolved in tetrahydrofuran (40 mL), and the solution was added dropwise to the above mixture. The resultant mixture was stirred at room temperature for 3 hours. The solid was removed through filtration and purified through silica gel chromatography, to thereby yield pppM-16.

To a reaction vessel, pppM-18 (30 mmol), methacrylic acid (39 mmol). , EDC (39 mmol), DMAP (6 mmol), and dichloromethane (50 mL) were added, and the mixture was stirred at room temperature for 2 hours. 2M hydrochloric acid (50 mL) was added thereto, and the organic layer was separated. The organic layer was washed twice with saturated aqueous sodium hydrogen carbonate (50 mL) and once with ultrapure water (50 mL), to thereby yield ppM-18.

To a reaction vessel, ppM-18 (20 mmol) and tetrahydrofuran (40 mL) were added, and the mixture was stirred at 0° C. for 30 minutes. Sodium hydroborate was added thereto, and the mixture was stirred at 0° C. for 1 hour. Saturated aqueous ammonium chloride (40 mL) was added, and extraction was conducted with ethyl acetate (100 mL), to thereby yield pM-18.

To a reaction vessel, pM-18 (13 mmol), a compound represented by the aforementioned formula (S-3) (10 mmol), EDC (13 mmol), DMAP (2 mmol), and dichloromethane (20 mL), were added, and the mixture was stirred at room temperature for 2 hours. 2M hydrochloric acid (20 mL) was added thereto, and the organic layer was separated. The organic layer was washed twice with saturated aqueous sodium hydrogen carbonate (20 mL) and once with ultrapure water (20 mL), and purified through silica gel column chromatography, to thereby yield monomer (M-18)

### [Synthesis Example 1-5] (Synthesis of Monomer (M-19))

Monomer (M-19) was synthesized through the following reaction scheme.

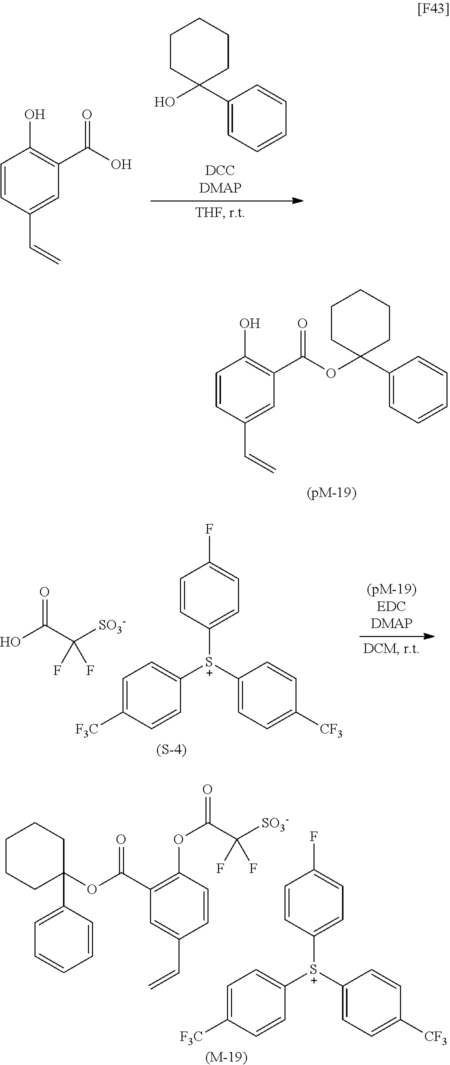

To a reaction vessel, 5-vinylsalicylic acid (40 mmol), 1-phenylcyclohexanol (80 mmol), and DMAP (8 mmol) were added, and the mixture was stirred at room temperature for 1 hour. DCC (44 mmol) was dissolved in tetrahydrofuran (40 mL), and the solution was added dropwise to the above mixture. The resultant mixture was stirred at room temperature for 3 hours. The solid was removed through filtration and purified through silica gel chromatography, to thereby yield pM-19.

To a reaction vessel, pM-19 (13 mmol), a compound represented by the aforementioned formula (S-4) (10 mmol), EDC (13 mmol), DMAP (2 mmol), and dichloromethane (20 mL) were added, and the mixture was stirred at room temperature for 2 hours. 2M hydrochloric acid (20 mL) was added thereto, and the organic layer was separated. The organic layer was washed twice with saturated aqueous sodium hydrogen carbonate (20 mL) and once with ultrapure water (20 mL), and purified through silica gel column chromatography, to thereby yield monomer (M-19).

### [Synthesis Example 1-6] (Synthesis of Monomer (M-20))

Monomer (M-20) was synthesized through the following reaction scheme.

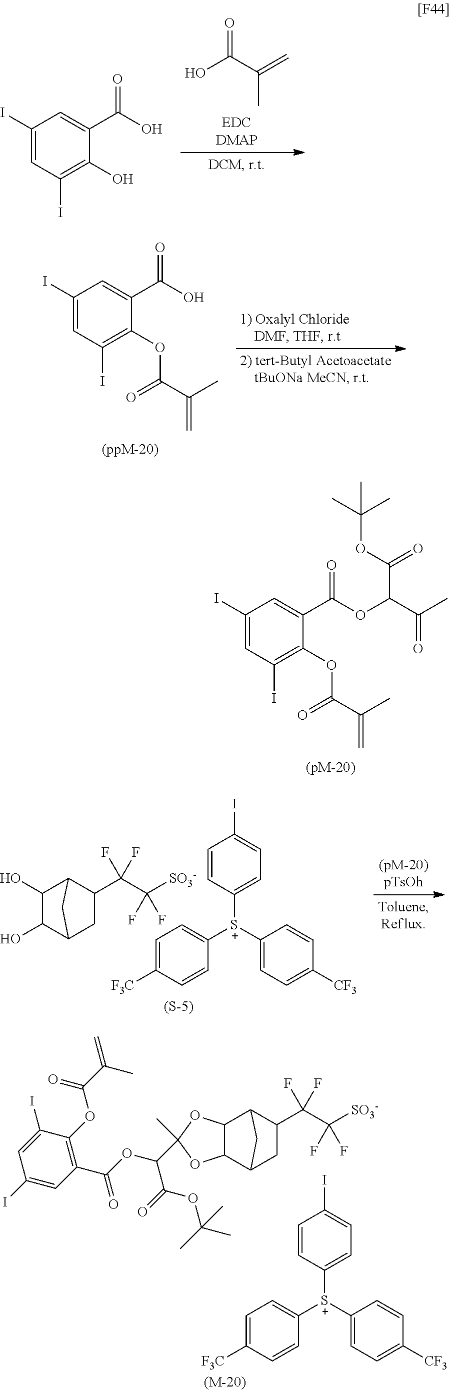

To a reaction vessel, 3,5-diiodosalicylic acid (30 mmol), methacrylic acid (39 mmol), EDC (39 mmol), DMAP (6 mmol), and dichloromethane (50 mL) were added, and the mixture was stirred at room temperature for 2 hours. 2M hydrochloric acid (50 mL) was added thereto, and the organic layer was separated. The organic layer was washed twice with saturated aqueous sodium hydrogen carbonate (50 mL) and once with ultrapure water (50 mL), and purified through silica gel column chromatography, to thereby yield ppM-20.

To a reaction vessel, ppM-20 (20 mmol), tetrahydrofuran (40 mL), and N,N-dimethylformamide (0.1 mL) were added, and the mixture was stirred. Oxalyl chloride (40 mmol) was added dropwise thereto, and the resultant mixture was stirred at room temperature for 1 hour. Subsequently, tert-butyl acetoacetate (30 mmol) and sodium tert-butoxide (30 mmol) were dissolved in acetonitrile (40 mL), and the solution was added dropwise thereto. Saturated aqueous ammonium chloride (50 mL) was added, and the organic layer was separated. The organic layer was washed with ultrapure water (50 mL) and purified through silica gel column chromatography, to thereby yield pM-20.

To a reaction vessel, a compound represented by the aforementioned formula (S-5) (20 mmol), pM-20 (21 mmol), pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-20).

### [Synthesis Example 1-7] (Synthesis of Monomer (M-21))

Monomer (M-21) was synthesized through the following reaction scheme.

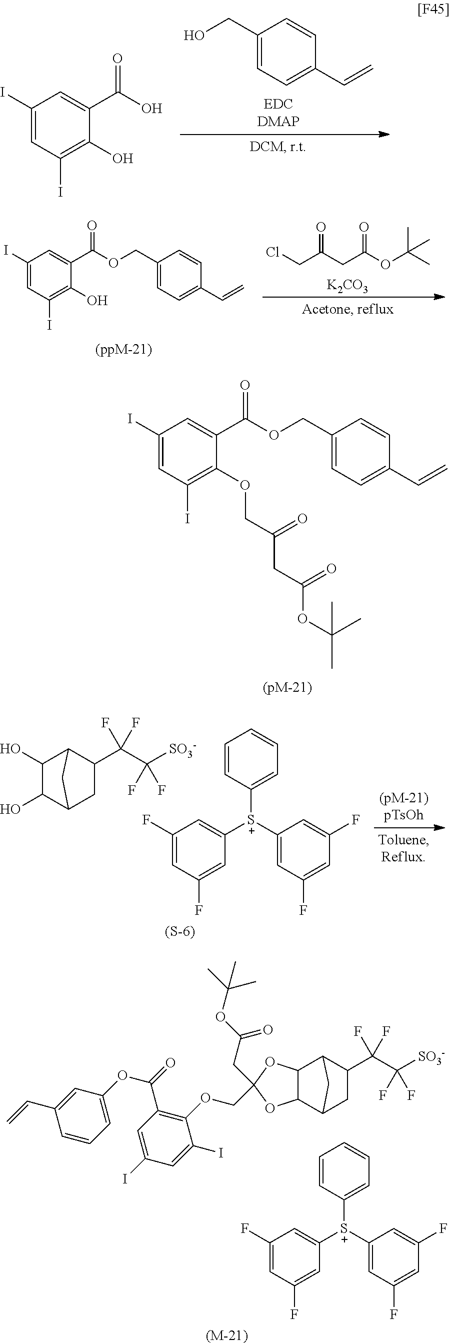

To a reaction vessel, 3,5-diiodosalicylic acid (30 mmol), 4-(hydroxymethyl) styrene (39 mmol), EDC (39 mmol), DMAP (6 mmol), and dichloromethane (50 mL) were added, and the mixture was stirred at room temperature for 2 hours. 2M hydrochloric acid (50 mL) was added thereto, and the organic layer was separated. The organic layer was washed twice with saturated aqueous sodium hydrogen carbonate (50 mL) and once with ultrapure water (50 mL), to thereby yield ppM-21.

To reaction vessel, ppM-21 (29.74 mmol), potassium carbonate (148.7 mmol), and acetone (100 mL) were added, and the mixture was stirred for 1 hour. Subsequently, tert-butyl 4-chloroacetoacetate (59.47 mmol) was added dropwise thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield pM-21.

To a reaction vessel, a compound represented by the aforementioned formula (S-6) (20 mmol), pM-21 (21 mmol), pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-21).

### [Synthesis Example 1-8] (Synthesis of Monomer (M-22))

Monomer (M-22) was synthesized through the following reaction scheme.

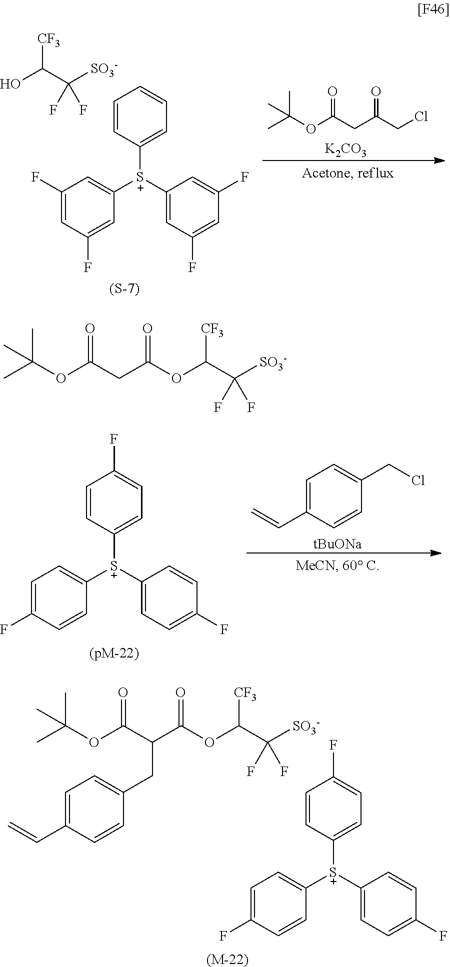

To a reaction vessel, a compound represented by the aforementioned formula (S-7) (29.74 mmol), potassium carbonate (148.7 mmol), and acetone (100 mL) were added, and the mixture was stirred for 1 hour. Subsequently, tert-butyl 4-chloroacetoacetate (59.47 mmol) was added dropwise thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield pM-22.

To a reaction vessel, pM-22 (20 mmol), sodium tert-butoxide (100 mmol), and acetonitrile (80 mL) were added, and the mixture was stirred for 1 hour. Subsequently, 4-(chloromethyl) styrene (40 mmol) was added dropwise to the mixture, and the resultant mixture was heated at 60° C. for 5 hours under stirring. The resultant mixture was cooled to room temperature, and the precipitated solid was removed through filtration. The filtrate was concentrated and purified through silica gel column chromatography, to thereby yield monomer (M-22).

### [Synthesis Examples 1-9 and 1-10] (Synthesis of Monomers (M-23) and (M-24)

The procedure of Synthesis Example 1-8 was repeated, except that appropriately selected substrates were used in Synthesis Example 1-8, to thereby synthesize monomers (M-23) and (M-24).

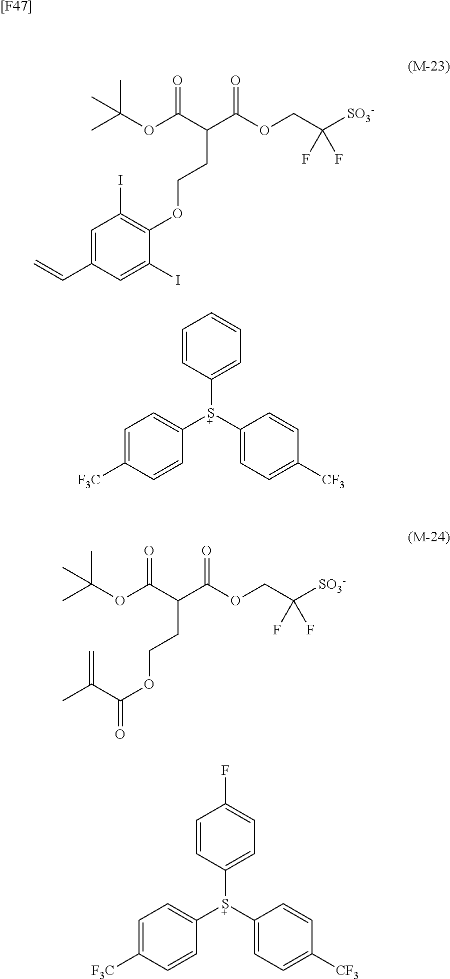

### [Synthesis Example 1-11] (Synthesis of Monomer (M-25))

Monomer (M-25) was synthesized through the following reaction scheme.

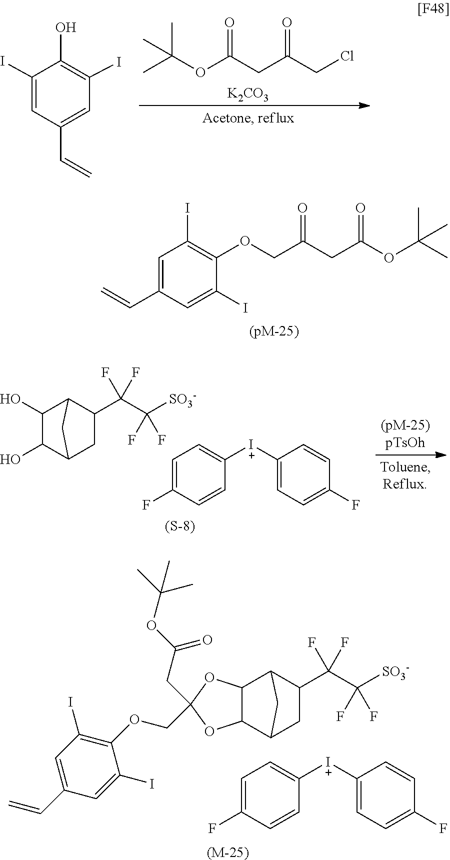

To a reaction vessel, 3, 5-diiodo-4-hydroxystyrene (29.74 mmol), potassium carbonate (148.7 mmol), and acetone (100 mL) were added, and the mixture was stirred for 1 hour. Subsequently, tert-butyl 4-chloroacetoacetate (59.47 mmol) was added dropwise thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield pM-25.

To a reaction vessel, a compound represented by the aforementioned formula (S-8) (20 mmol), pM-25 (21 mmol), pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-25).

### [Synthesis Example 1-12] (Synthesis of Monomer (M-26))

Monomer (M-26) was synthesized through the following

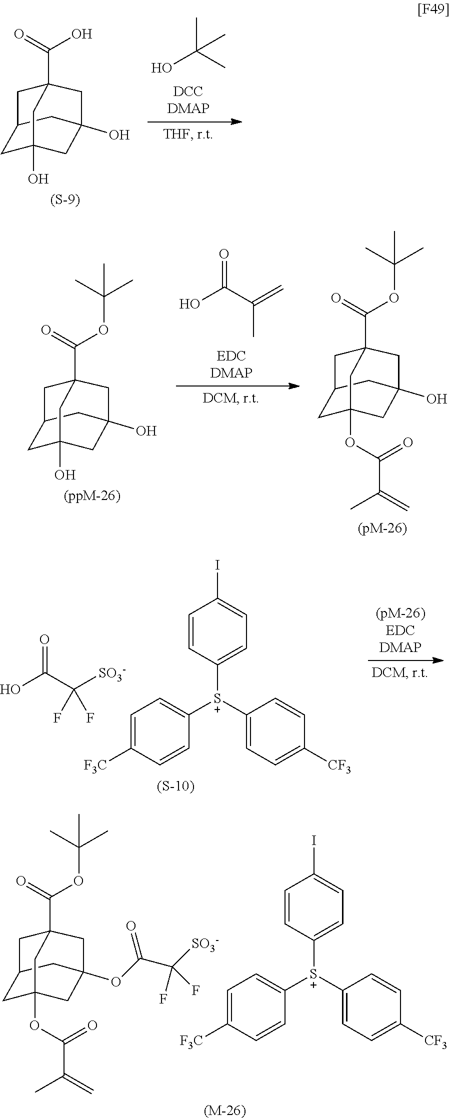

To a reaction vessel, a compound represented by the aforementioned formula (S-9) (40 mmcl), tert-butanol (200 mmci), and DMAP (8 mmcl) were added, and the mixture was stirred for 1 hour. DCC (44 mmol) was dissolved in tetrahydrofuran (40 mL), and the solution was added dropwise to the above mixture. The resultant mixture was stirred at room temperature for 3 hours. The solid was removed through filtration and purified through silica gel chromatography, to thereby yield ppM-26.

To a reaction vessel, ppM-26 (30 mmol), methacrylic acid (39 mmcl), EDC (39 mmol), DMAP (6 mmol), and dichloromethane (20 mL) were added, and the mixture was stirred at room temperature for 2 hours. 2M hydrochloric acid (20 mL) was added thereto, and the organic layer was separated. The organic layer was washed twice with saturated aqueous sodium hydrogen carbonate (20 mL) and once with ultrapure water (20 mL), to thereby yield pM-26.

To a reaction vessel, pM-26 (13 mmol), a compound represented by the aforementioned formula (5-10) (10 mmol), EDC (13 mmol), DMAP (2 mmol), and dichloromethane (20 mL) were added, and the mixture was stirred at room temperature for 2 hours. 2M hydrochloric acid (20 mL) was added thereto, and the organic layer was separated. The organic layer was washed twice with saturated aqueous sodium hydrogen carbonate (20 mL) and once with ultrapure water (20 mL), and purified through silica gel column chromatography, to thereby yield monomer (M-26).

### [Synthesis Example 1-13] (Synthesis of Monomer (M-27))

The procedure of Synthesis Example 1-12 was repeated, except that appropriately selected substrates were used in Synthesis Example 1-12, to thereby synthesize monomer (M-27).

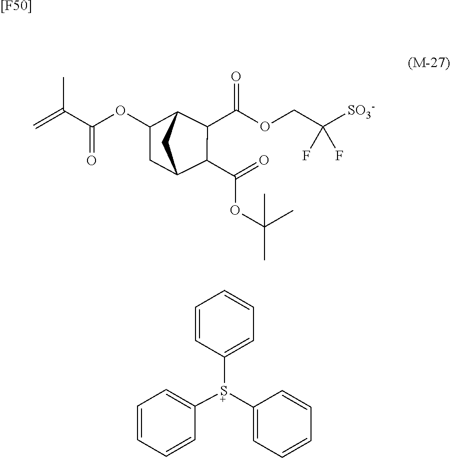

### [Synthesis Example 1-14] (Synthesis of Monomer (M-28))

Monomer (M-28) was synthesized through the following reaction scheme.

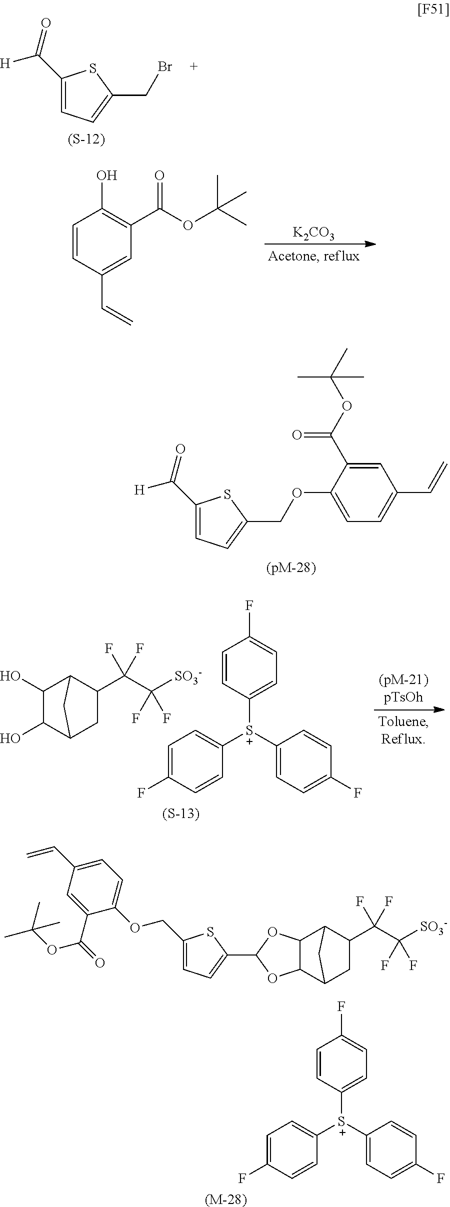

To a reaction vessel, tert-butyl 5-vinylsalicylate (29.74 mmol), potassium carbonate (148.7 mmcl), and acetone (100 mL) were added, and the mixture was stirred for 1 hour. Subsequently, a compound represented by the aforementioned formula (S-12) (59.47 mmol) was added thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield pM-28.

To a reaction vessel, a compound represented by the aforementioned formula (S-13) (20 mmol), pM-28 (21 mmcl), pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-28).

### [Synthesis Example 1-15] (Synthesis of Monomer (M-29))

The procedure of Synthesis Example 1-14 was repeated, except that appropriately selected substrates were used in Synthesis Example 1-14, to thereby synthesize monomer (M-29)

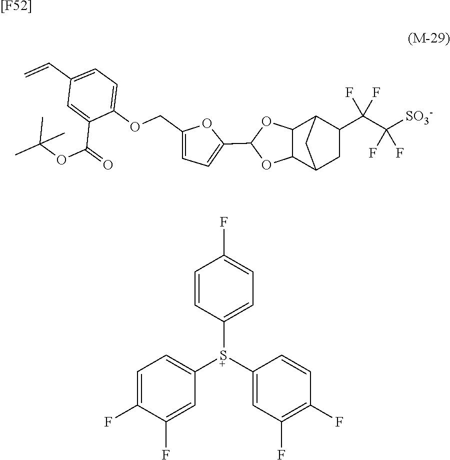

### [Synthesis Example 1-16] (Synthesis of Monomer (M-30))

Monomer (M-30) was synthesized through the following reaction scheme.

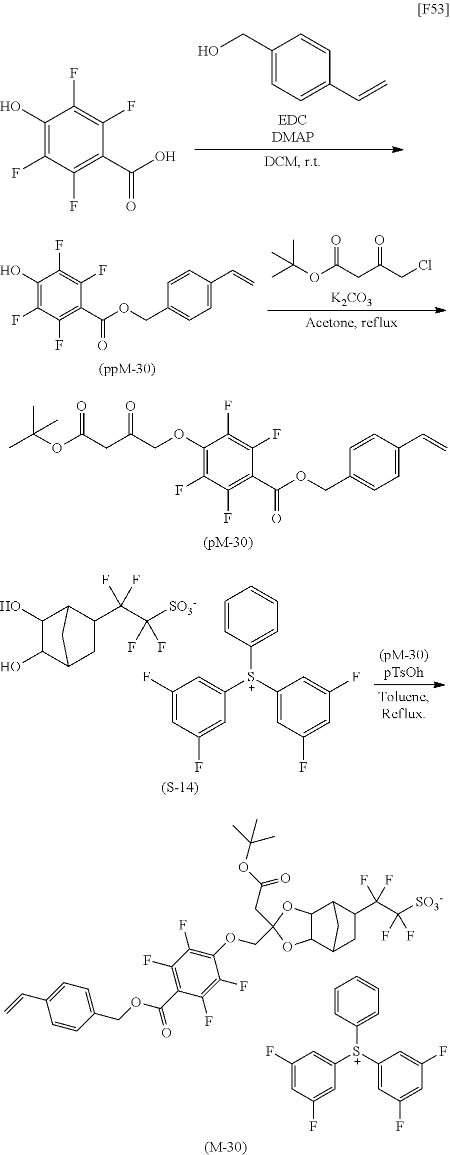

To a reaction vessel, 2,3,5,6-tetrafluoro-4-hydroxybenzoic acid (30 mmcl), 4-(hydroxymethyl)styrene (39 mmcl), EDC (39 mmcl), DMAP (6 mmcl), and dichloromethane (50 mL) were added, and the mixture was stirred for 2 hours. 2M hydrochloric acid (50 mL) was added thereto, and the organic layer was separated. The organic layer was washed twice with saturated aqueous sodium hydrogen carbonate (50 mL) and once with ultrapure water (50 mL), to thereby yield ppM-30.

To a reaction vessel, ppM-30 (29.74 mmcl), potassium carbonate (148.7 mmcl), and acetone (100 mL) were added, and the mixture was stirred for 1 hour. Subsequently, tert-butyl 4-chloroacetoacetate (59.47 mmcl) was added dropwise thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield pM-30.

To a reaction vessel, a compound represented by the aforementioned formula (S-14) (20 mmol), pM-30 (21 mmol), pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-30).

### [Synthesis Example 1-17] (Synthesis of Monomer (M-31))

Monomer (M-31) was synthesized through the following reaction scheme.

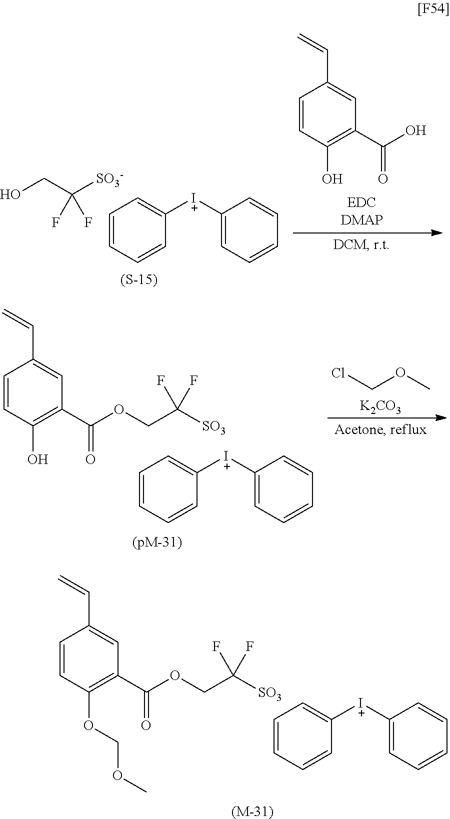

To a reaction vessel, 5-vinylsalicylic acid (30 mmol), a compound represented by the aforementioned formula (S-15) (39 mmol), EDC (39 mmol), DMAP (6 mmol), and dichloromethane (50 mL) were added, and the mixture was stirred for 2 hours. 2M hydrochloric acid (50 mL) was added thereto, and the organic layer was separated. The organic layer was washed twice with saturated aqueous sodium hydrogen carbonate (50 mL) and once with ultrapure water (50 mL), to thereby yield pM-31.

To a reaction vessel, pM-31 (29.74 mmol), potassium carbonate (148.7 mmol), and acetone (100 mL) were added, and the mixture was stirred for 1 hour. Subsequently, chloromethyl methyl ether (59.47 mmol) was added thereto, and the contents were heated under reflux and stirring conditions for 5 hours. The resultant mixture was cooled to room temperature, and the precipitated solid was removed through filtration. The filtrate was concentrated and purified through silica gel column chromatography, to thereby yield monomer (M-31).

### [Synthesis Example 1-18] (Synthesis of Monomer (M-32))

Monomer (M-32) was synthesized through the following reaction scheme.

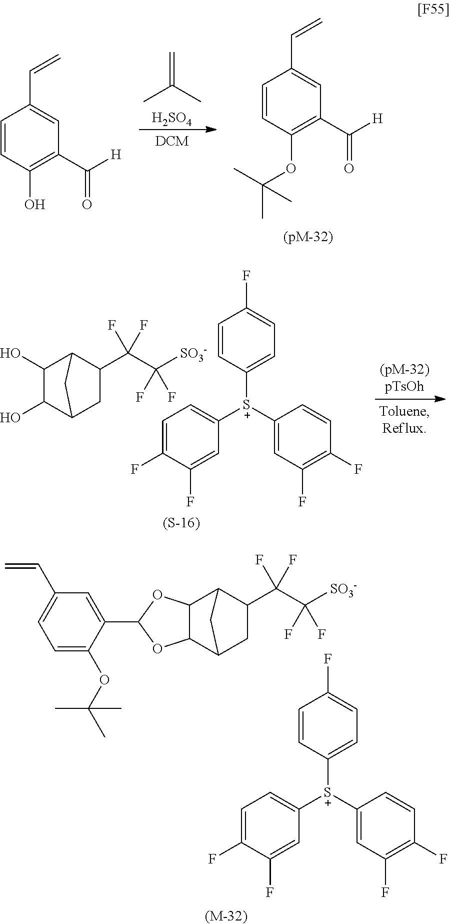

To a reaction vessel, 5-vinylsalicyladhyhde (30 mmol), dichloromethane (100 mL), and concentrated sulfuric acid (2 mL) were added. Isobutene gas (30 mL) was introduced into the mixture, and the resultant mixture was stirred at room temperature for 6 hours. Saturated aqueous sodium hydrogen carbonate (80 mL) was added thereto, to thereby isolate the organic layer. The organic layer was dried over sodium sulfate, and the solvent was removed. The product was purified through silica gel chromatography, to thereby yield monomer (pM-32).

To a reaction vessel, a compound represented by the aforementioned formula (S-16) (20 mmol), pM-32 (21 mmol) pTsOH (4 mmol), and toluene (100 mL) were added. A Dien-Stark tube was attached to the reaction vessel, and the contents of the reaction vessel were stirred for 4 hours under reflux and heating conditions. Saturated aqueous sodium hydrogen carbonate (50 mL) was added thereto, to thereby isolate the organic layer. The organic layer was dried over sodium sulfate, and the solvent was removed. The product was purified through silica gel chromatography, to thereby yield monomer (M-32).

### 2. Synthesis of Base Resins

### [Synthesis Examples 2-1 to 2-35] Synthesis of Base Resins

Monomers of interest were chosen at compositional proportions shown in the following Table 1 and copolymerized in tetrahydrofuran (THF) as a solvent. Each copolymerized product was crystallized in methanol and repeatedly washed with hexane, then isolated and dried, to thereby yield base resins A-1 to A-35. The Mw and the dispersity (Mw/Mn) of each resin were determined through gel permeation chromatography (GPC) with GPC columns (G2000HXL×2, G3000HXL×1, and G4000HXL×1) (products of Tosoh Corp.) under the following conditions.
Eluent: tetrahydrofuran (product of FUJIFILM Wako Pure Chemical Corporation)
Flow rate: 1.0 mL/min
Sample concentration: 1.0 mass %
Sample injection: 100 μL
Column temperature: 40° C.
Detector: differential refractometer
Standard: monodispersed polystyrene

TABLE 1

| | | Monomer for | | Monomer for | | Monomer for | | | |
|---|---|---|---|---|---|---|---|---|---|
| | | forming first | | forming second | | forming third | | | |
| | | structural unit | | structural unit | | structural unit | | | |
| | Polymer | | Amount | | Amount | | Amount | | |
| | Name | Type | (mol %) | Type | (mol %) | Type | (mol %) | Mw | Mw/Mn |
| Synthesis Example 2-1 | A-1 | M-15 | 15 | M-1 | 30 | M-5 | 55 | 6000 | 1.6 |
| Synthesis Example 2-2 | A-2 | M-15 | 15 | M-2 | 30 | M-5 | 55 | 6300 | 1.7 |
| Synthesis Example 2-3 | A-3 | M-15 | 15 | M-3 | 30 | M-5 | 55 | 6000 | 1.6 |
| Synthesis Example 2-4 | A-4 | M-15 | 15 | M-4 | 30 | M-5 | 55 | 5500 | 1.7 |
| Synthesis Example 2-5 | A-5 | M-15 | 15 | M-1 | 30 | M-6 | 55 | 6100 | 1.6 |
| Synthesis Example 2-6 | A-6 | M-15 | 15 | M-1 | 30 | M-7 | 55 | 6100 | 1.7 |
| Synthesis Example 2-7 | A-7 | M-15 | 15 | M-1 | 30 | M-8 | 55 | 6300 | 1.5 |
| Synthesis Example 2-8 | A-8 | M-15 | 15 | M-1 | 30 | M-9 | 55 | 6200 | 1.6 |
| Synthesis Example 2-9 | A-9 | M-15 | 15 | M-1 | 30 | M-10 | 55 | 5900 | 1.5 |
| Synthesis Example 2-10 | A-10 | M-15 | 15 | M-1 | 30 | M-11 | 55 | 5600 | 1.6 |
| Synthesis Example 2-11 | A-11 | M-15 | 15 | M-1 | 30 | M-12 | 55 | 6900 | 1.6 |
| Synthesis Example 2-12 | A-12 | M-15 | 15 | M-1 | 30 | M-13 | 55 | 6300 | 1.7 |
| Synthesis Example 2-13 | A-13 | M-15 | 15 | M-1 | 30 | M-14 | 55 | 5900 | 1.5 |
| Synthesis Example 2-14 | A-14 | M-16 | 15 | M-1 | 30 | M-5 | 55 | 6900 | 1.6 |
| Synthesis Example 2-15 | A-15 | M-17 | 15 | M-1 | 30 | M-5 | 55 | 6800 | 1.7 |
| Synthesis Example 2-16 | A-16 | M-18 | 15 | M-1 | 30 | M-5 | 55 | 6700 | 1.5 |
| Synthesis Example 2-17 | A-17 | M-19 | 15 | M-1 | 30 | M-5 | 55 | 5800 | 1.5 |
| Synthesis Example 2-18 | A-18 | M-20 | 15 | M-1 | 30 | M-5 | 55 | 5600 | 1.6 |
| Synthesis Example 2-19 | A-19 | M-21 | 15 | M-1 | 30 | M-5 | 55 | 5400 | 1.6 |
| Synthesis Example 2-20 | A-20 | M-22 | 15 | M-1 | 30 | M-5 | 55 | 5900 | 1.6 |
| Synthesis Example 2-21 | A-21 | M-23 | 15 | M-1 | 30 | M-5 | 55 | 5700 | 1.7 |
| Synthesis Example 2-22 | A-22 | M-24 | 15 | M-1 | 30 | M-5 | 55 | 6300 | 1.5 |
| Synthesis Example 2-23 | A-23 | M-25 | 15 | M-1 | 30 | M-5 | 55 | 6700 | 1.6 |
| Synthesis Example 2-24 | A-24 | M-26 | 15 | M-1 | 30 | M-5 | 55 | 6000 | 1.6 |
| Synthesis Example 2-25 | A-25 | M-27 | 15 | M-1 | 30 | M-5 | 55 | 5700 | 1.7 |
| Synthesis Example 2-26 | A-26 | M-28 | 15 | M-1 | 30 | M-5 | 55 | 6400 | 1.5 |
| Synthesis Example 2-27 | A-27 | M-29 | 15 | M-1 | 30 | M-5 | 55 | 5600 | 1.7 |
| Synthesis Example 2-28 | A-28 | M-30 | 15 | M-1 | 30 | M-5 | 55 | 6400 | 1.6 |
| Synthesis Example 2-29 | A-29 | M-31 | 15 | M-1 | 30 | M-5 | 55 | 5900 | 1.5 |
| Synthesis Example 2-30 | A-30 | M-32 | 15 | M-1 | 30 | M-5 | 55 | 6300 | 1.7 |
| Synthesis Example 2-31 | A-31 | M-15 | 10 | M-1 | 35 | M-5 | 55 | 6800 | 1.5 |
| Synthesis Example 2-32 | A-32 | M-15 | 5 | M-1 | 40 | M-5 | 55 | 6100 | 1.6 |
| Synthesis Example 2-33 | A-33 | M-15 | 20 | M-1 | 25 | M-5 | 55 | 6100 | 1.5 |
| Synthesis Example 2-34 | A-34 | M-33 | 15 | M-1 | 30 | M-5 | 55 | 5400 | 1.5 |
| Synthesis Example 2-35 | A-35 | M-34 | 15 | M-1 | 30 | M-5 | 55 | 5600 | 1.5 |

Monomers employed in polymerization are as follows.

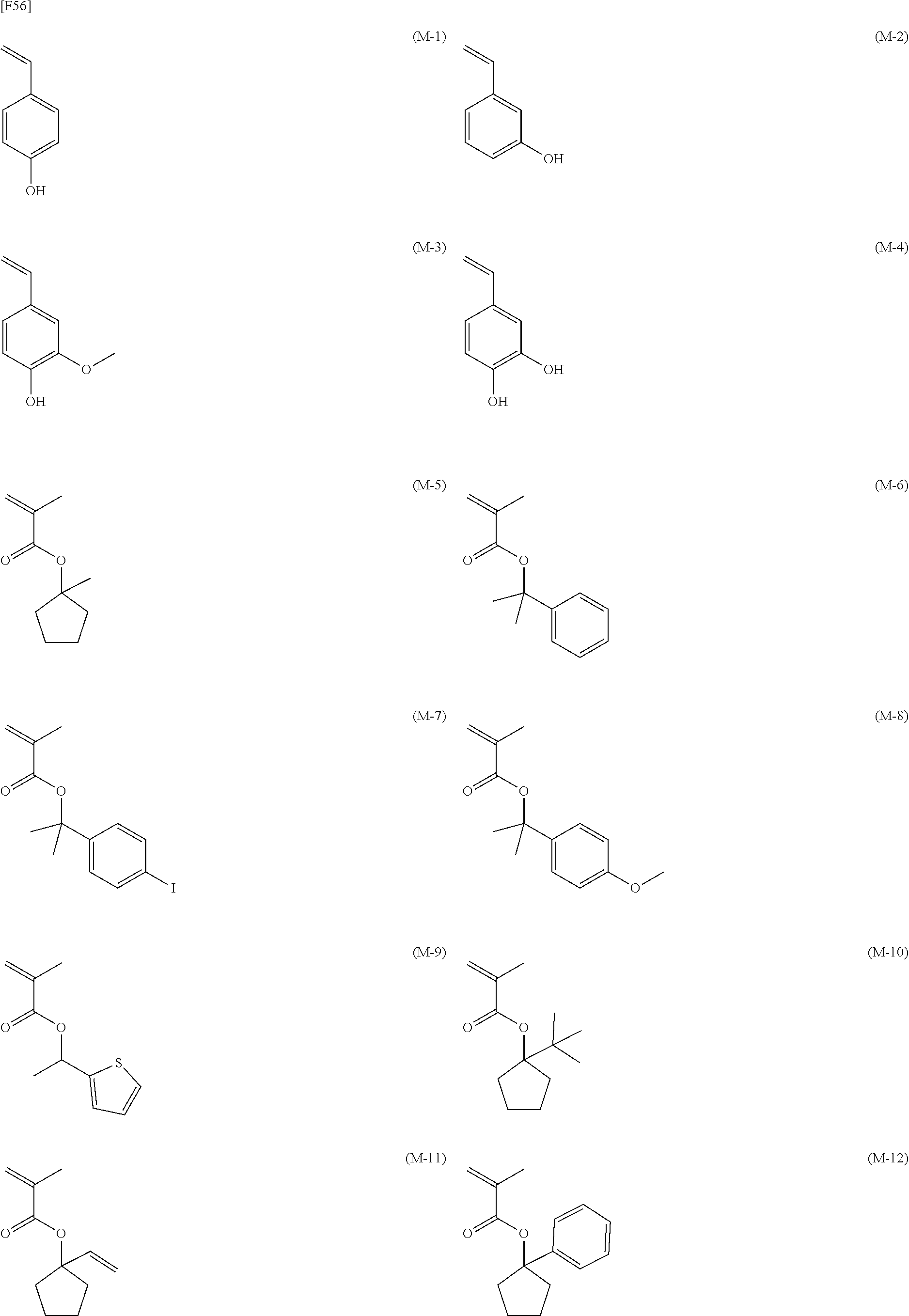

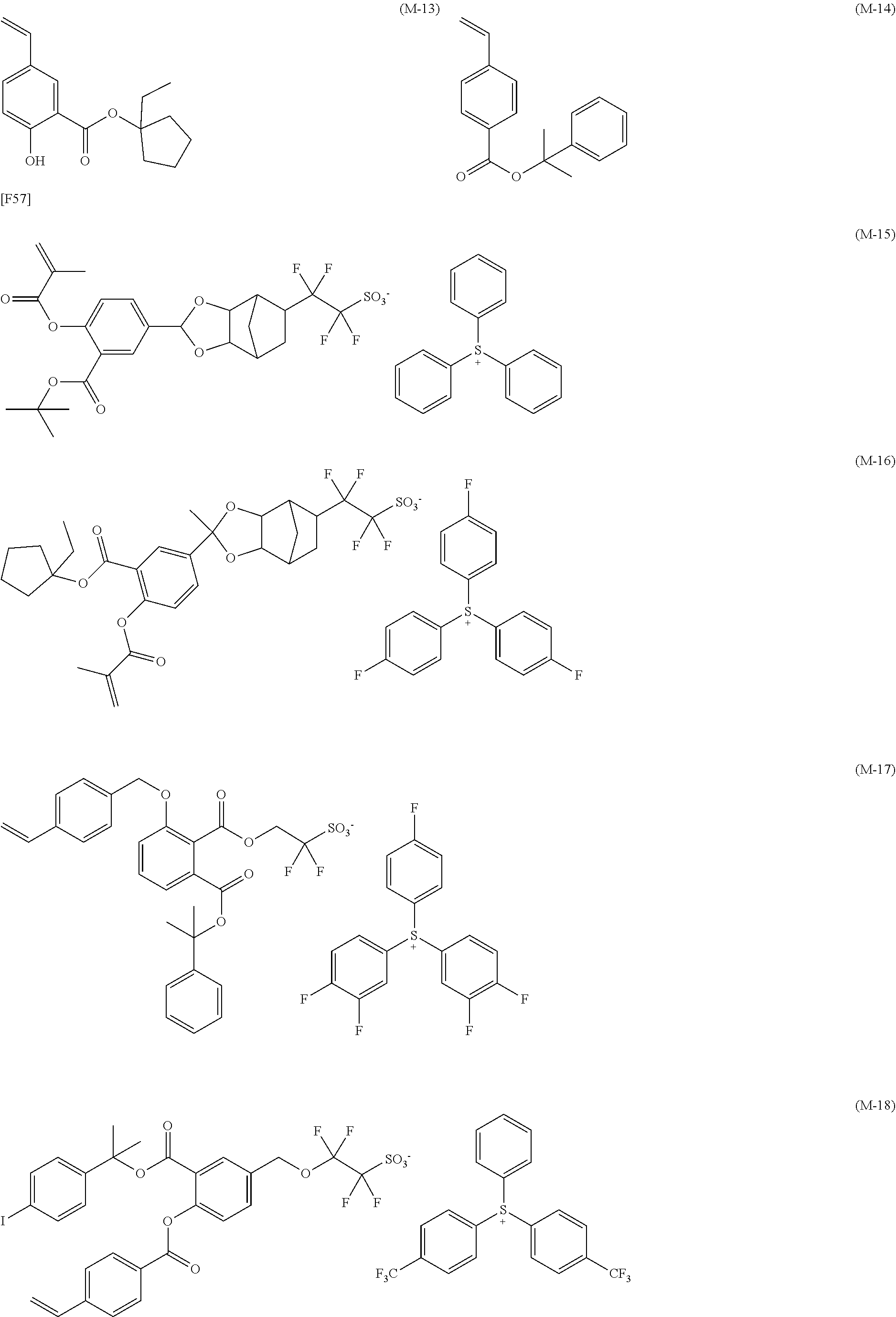

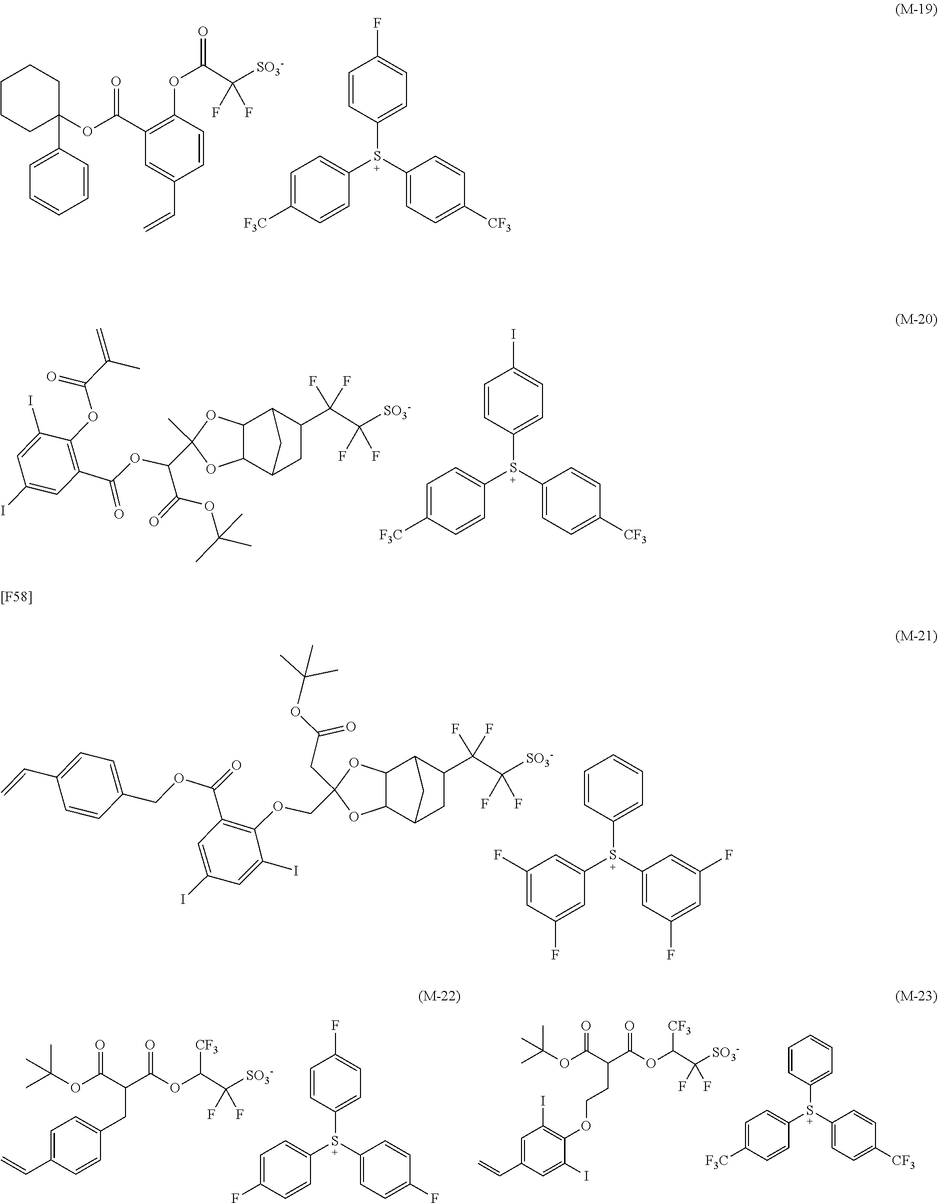

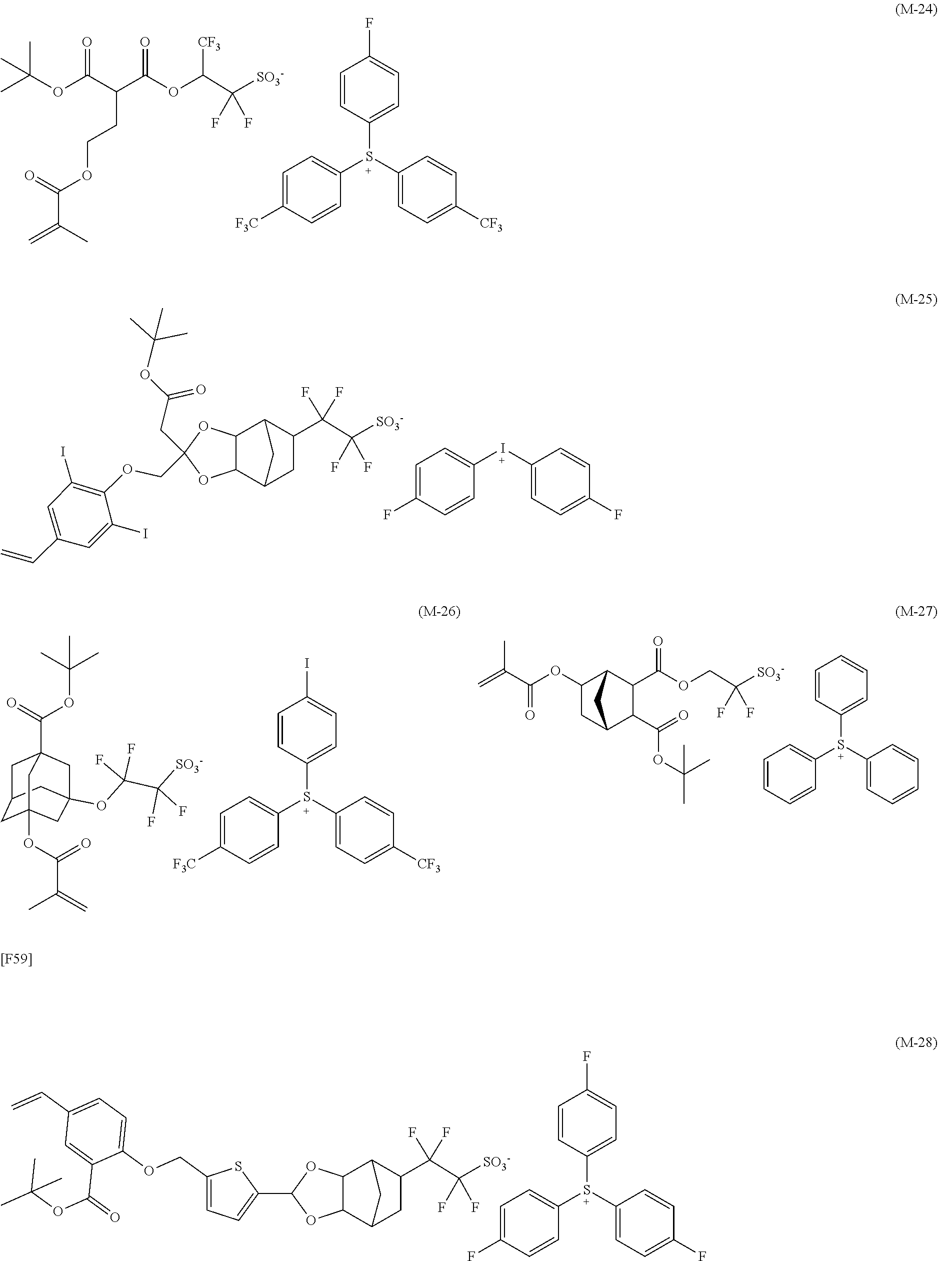

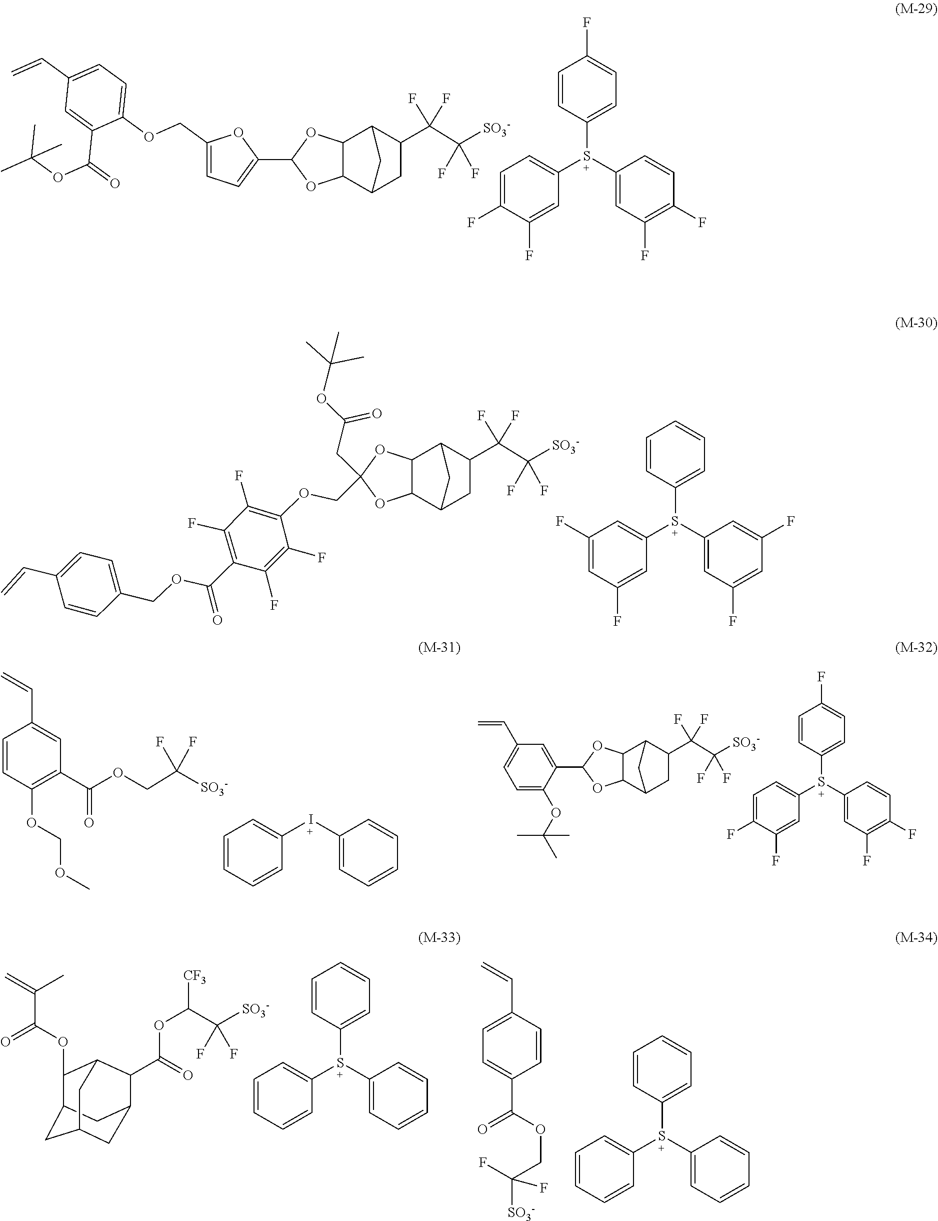

### 4. Preparation and Evaluation of Radiation-Sensitive Resin Compositions

Compounds employed in preparation of radiation-sensitive resin compositions are as follows.

### <[A] Base Resin>

A-1 to A-35: polymers obtained in Synthesis Examples 2-1 to 2-35.

### <[B] Radiation-Sensitive Acid-Generating Agent>

B-1 to B-4: compounds represented by the following formulas (B-1) to (B-4), respectively.

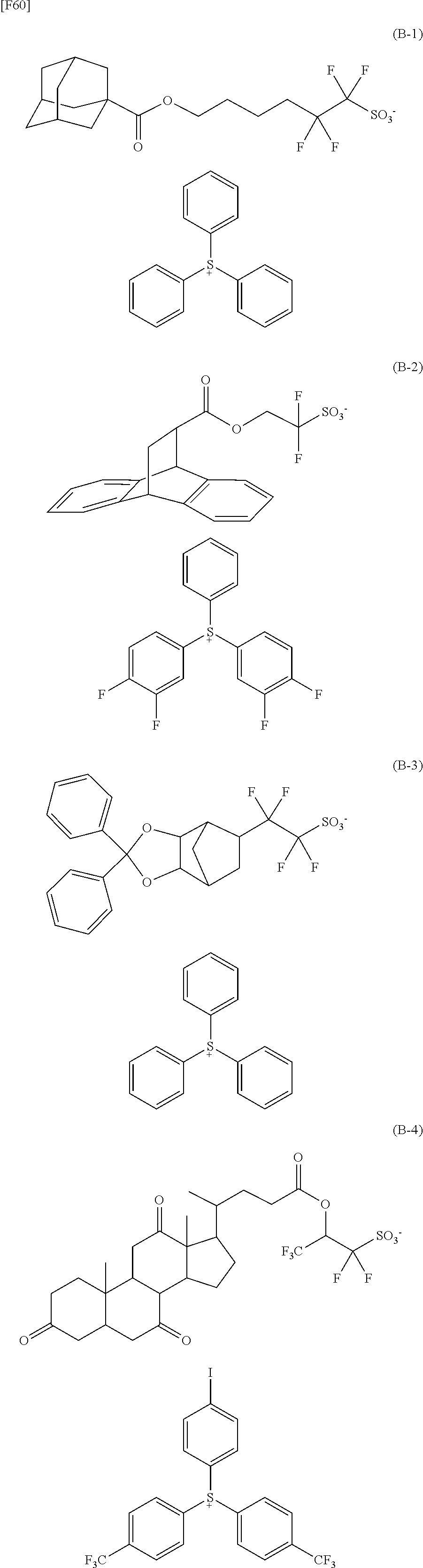

### <[Z] Acid Diffusion Control Agent>

Z-1 to Z-4: compounds represented by the following formulas (Z-1) to (Z-4), respectively.

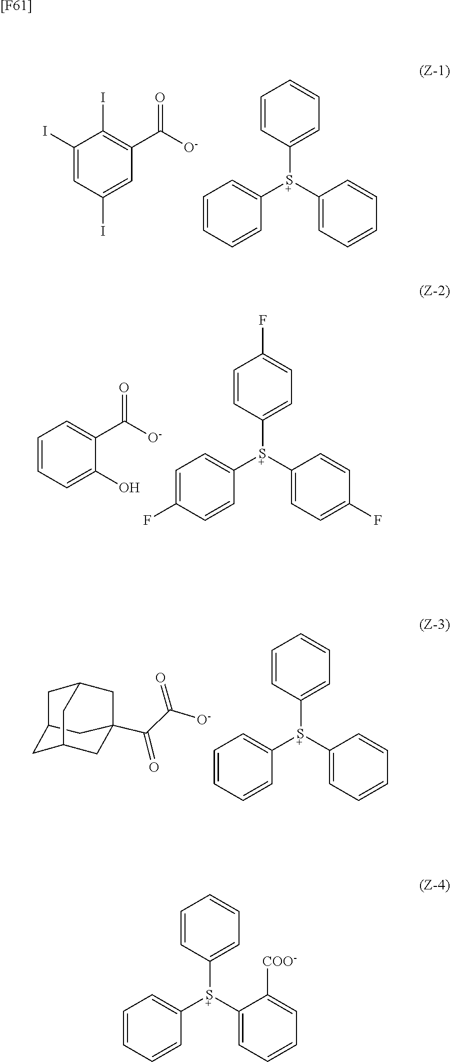

### [D] Solvent

D-1: propylene glycol monomethyl ether acetate
D-2: propylene glycol 1-monomethyl ether

### Preparation of Radiation-Sensitive Compositions

### Example 1

The polymer (A-1) serving the base resin [A](100 parts by mass), a compound (Z-1) serving as the acid diffusion control agent [Z] (20 mol %, with respect to the amount of the monomer for forming the first structural unit in 100 parts by mass of the polymer (A-1)), and the compound (D-1) serving as the solvent [D] (2,000 parts by mass), and a compound (D-2) (4,800 parts by mass) were blended together, to thereby prepare a radiation-sensitive composition (R-1).

### Examples 2 to 40 and Comparative Examples 1 and 2

The procedure of Example 1 was repeated, except that the components and proportions listed in the following Table 2 were used, to thereby prepare radiation-sensitive compositions (R-2) to (R-40) and (CR-1) and (CR-2) Notably, in Examples 34 to 37, the acid diffusion control agent [Z] was added in an amount of 20 mol %, with respect to the total amount of the monomer forming the first structural unit included in the base resin [A] in an amount of 100 parts by mass and the amount of the radiation-sensitive acid-generating agent [B]

TABLE 2

| | | | | | | Acid diffusion | | | |
|---|---|---|---|---|---|---|---|---|---|
| | | | | | | control agent | | | |
| | | | | | | | ratio (mol %) | | |
| | | | | Radiation-sensitive | | | with respect | | |
| | | Base resin | | acid-generating agent | | | to amount of | Solvent | |
| | Radiation- | | Part(s) | | Part(s) | | acig- | | Part(s) |
| | sensitive | | by | | by | | generating | | by |
| | composition | Type | mass | Type | mass | Type | agent | Type | mass |
| Example 1 | R-1 | A-1 | 100 | — | — | Z-1 | 20 | D-1/D-2 | 2000/4800 |
| Example 2 | R-2 | A-2 | 100 | — | — | Z-1 | 20 | D-1/D-2 | 2000/4800 |
| Example 3 | R-3 | A-3 | 100 | — | — | Z-1 | 20 | D-1/D-2 | 2000/4800 |
| Example 4 | R-4 | A-4 | 100 | — | — | Z-1 | 20 | D-1/D-2 | 2000/4800 |
| Example 5 | R-5 | A-5 | 100 | — | — | Z-1 | 20 | D-1/D-2 | 2000/4800 |
| Example 6 | R-6 | A-6 | 100 | — | — | Z-1 | 20 | D-1/D-2 | 2000/4800 |
| Example 7 | R-7 | A-7 | 100 | — | — | Z-1 | 20 | D-1/D-2 | 2000/4800 |
| Example 8 | R-8 | A-8 | 100 | — | — | Z-1 | 20 | D-1/D-2 | 2000/4800 |
| Example 9 | R-9 | A-9 | 100 | — | — | Z-1 | 20 | D-1/D-2 | 2000/4800 |
| Example 10 | R-10 | A-10 | 100 | — | — | Z-1 | 20 | D-1/D-2 | 2000/4800 |
| Example 11 | R-11 | A-11 | 100 | — | — | Z-1 | 20 | D-1/D-2 | 2000/4800 |
| Example 12 | R-12 | A-12 | 100 | — | — | Z-1 | 20 | D-1/D-2 | 2000/4800 |
| Example 13 | R-13 | A-13 | 100 | — | — | Z-1 | 20 | D-1/D-2 | 2000/4800 |
| Example 14 | R-14 | A-14 | 100 | — | — | Z-1 | 20 | D-1/D-2 | 2000/4800 |
| Example 15 | R-15 | A-15 | 100 | — | — | Z-1 | 20 | D-1/D-2 | 2000/4800 |
| Example 16 | R-16 | A-16 | 100 | — | — | Z-1 | 20 | D-1/D-2 | 2000/4800 |
| Example 17 | R-17 | A-17 | 100 | — | — | Z-1 | 20 | D-1/D-2 | 2000/4800 |
| Example 18 | R-18 | A-18 | 100 | — | — | Z-1 | 20 | D-1/D-2 | 2000/4800 |
| Example 19 | R-19 | A-19 | 100 | — | — | Z-1 | 20 | D-1/D-2 | 2000/4800 |
| Example 20 | R-20 | A-20 | 100 | — | — | Z-1 | 20 | D-1/D-2 | 2000/4800 |
| Example 21 | R-21 | A-21 | 100 | — | — | Z-1 | 20 | D-1/D-2 | 2000/4800 |
| Example 22 | R-22 | A-22 | 100 | — | — | Z-1 | 20 | D-1/D-2 | 2000/4800 |
| Example 23 | R-23 | A-23 | 100 | — | — | Z-1 | 20 | D-1/D-2 | 2000/4800 |
| Example 24 | R-24 | A-24 | 100 | — | — | Z-1 | 20 | D-1/D-2 | 2000/4800 |
| Example 25 | R-25 | A-25 | 100 | — | — | Z-1 | 20 | D-1/D-2 | 2000/4800 |
| Example 26 | R-26 | A-26 | 100 | — | — | Z-1 | 20 | D-1/D-2 | 2000/4800 |
| Example 27 | R-27 | A-27 | 100 | — | — | Z-1 | 20 | D-1/D-2 | 2000/4800 |
| Example 28 | R-28 | A-28 | 100 | — | — | Z-1 | 20 | D-1/D-2 | 2000/4800 |
| Example 29 | R-29 | A-29 | 100 | — | — | Z-1 | 20 | D-1/D-2 | 2000/4800 |
| Example 30 | R-30 | A-30 | 100 | — | — | Z-1 | 20 | D-1/D-2 | 2000/4800 |
| Example 31 | R-31 | A-31 | 100 | — | — | Z-1 | 20 | D-1/D-2 | 2000/4800 |
| Example 32 | R-32 | A-32 | 100 | — | — | Z-1 | 20 | D-1/D-2 | 2000/4800 |
| Example 33 | R-33 | A-33 | 100 | — | — | Z-1 | 20 | D-1/D-2 | 2000/4800 |
| Example 34 | R-34 | A-1 | 100 | B-1 | 10 | Z-1 | 20 | D-1/D-2 | 2000/4800 |
| Example 35 | R-35 | A-1 | 100 | B-2 | 10 | Z-1 | 20 | D-1/D-2 | 2000/4800 |
| Example 36 | R-36 | A-1 | 100 | B-3 | 10 | Z-1 | 20 | D-1/D-2 | 2000/4800 |
| Example 37 | R-37 | A-1 | 100 | B-4 | 10 | Z-1 | 20 | D-1/D-2 | 2000/4800 |
| Example 38 | R-38 | A-1 | 100 | — | — | Z-2 | 20 | D-1/D-2 | 2000/4800 |
| Example 39 | R-39 | A-1 | 100 | — | — | Z-3 | 20 | D-1/D-2 | 2000/4800 |
| Example 40 | R-40 | A-1 | 100 | — | — | Z-4 | 20 | D-1/D-2 | 2000/4800 |
| Comparative | CR-1 | A-34 | 100 | — | — | Z-1 | 20 | D-1/D-2 | 2000/4800 |
| Example 1 | | | | | | | | | |
| Comparative | CR-2 | A-35 | 100 | — | — | Z-1 | 20 | D-1/D-2 | 2000/4800 |
| Example 2 | | | | | | | | | |

### <Formation of Resist Pattern>

A 12-inch silicon wafer having an under-layer film having a thickness of 40 nm (AL-412, product of Brewer Science, Inc.) was used. Onto the surface of the wafer, each of the above-prepared radiation-sensitive compositions was applied by means of a spin coater (CLEAN TRACK ACT12, product of Tokyo Electron Ltd.). The wafer was subjected to soft baking at 130° C. for 60 seconds and then cooled at 23° C. for 30 seconds, to thereby form a resist film having a thickness of 50 nm. Subsequently, the resist film was irradiated with EUV light by means of an EUV exposure device (NXE3300, product of ASML, NA=0.33, lighting condition: Conventional s=0.89, and mask: imecDEFECT32FFR02). Thereafter, the resist film was subjected to PEB (post exposure baking) at 110° C. for 60 seconds. Then, development was performed by use of 2.38 mass % aqueous TMAH at 23° C. for 30 seconds, to thereby form a positive-type line-and-space pattern (32 nm).

### <Evaluation>

Each of the formed resist patterns was evaluated in terms of sensitivity, LWR performance, and process margin of the radiation-sensitive composition, through the below-mentioned measurement procedures. Notably, dimensions of the resist pattern were measured under a scanning electron microscope (CG-4100, product of Hitachi High Technology Co., Ltd.). Table 3 shows the results of evaluation.

### [Sensitivity]

In the aforementioned “Formation of resist pattern,” a dose which can form a line-and-space pattern having a width of 32 nm was employed as an optimum dose, and the sensitivity was evaluated by the optimum dose (unit: mJ/cm²). The lower the value of the dose, the higher the sensitivity, the smaller the dose for achieving formation of a resist pattern of interest, which is preferred.

### [LWR Performance]

Each of the above-formed resist patterns was observed from above under the aforementioned scanning electron microscope, and line width was measured at 50 points selected at random. From the measurements, variation in width (30) was determined to serve as LWR performance (unit: nm). The smaller the variation in LWR value, the smaller the variation in line width (i.e., the more excellent the LWR performance).

### [Process Margin (Min CD)]

in the above formation of the resist pattern, the line width was measured while the dose of exposure decreased. The minimum line width not causing breakage and collapse of a line was employed as Min CD (unit: nm). The smaller the value of Min CD, the wider the process margin for forming the line, which is preferred.

TABLE 3

| | Radiation- | | | |
|---|---|---|---|---|
| | sensitive | Sensitivity | LWR | Min CD |
| | composition | (m J/cm²) | (nm) | (nm) |
| Example 1 | R-1 | 28 | 3.3 | 13.2 |
| Example 2 | R-2 | 28 | 3.1 | 13.6 |
| Example 3 | R-3 | 27 | 3.2 | 13.1 |
| Example 4 | R-4 | 26 | 3.1 | 13.4 |
| Example 5 | R-5 | 28 | 3.2 | 13.4 |
| Example 6 | R-6 | 27 | 3.0 | 13.7 |
| Example 7 | R-7 | 26 | 3.1 | 13.7 |
| Example 8 | R-8 | 26 | 2.9 | 13.9 |
| Example 9 | R-9 | 27 | 3.2 | 13.7 |
| Example 10 | R-10 | 25 | 3.2 | 13.0 |
| Example 11 | R-11 | 25 | 3.0 | 13.5 |
| Example 12 | R-12 | 27 | 3.1 | 13.3 |
| Example 13 | R-13 | 26 | 3.2 | 13.5 |
| Example 14 | R-14 | 24 | 2.9 | 13.0 |
| Example 15 | R-15 | 26 | 3.0 | 12.9 |
| Example 16 | R-16 | 24 | 2.9 | 12.9 |
| Example 17 | R-17 | 23 | 2.8 | 12.8 |
| Example 18 | R-18 | 22 | 2.7 | 12.6 |
| Example 19 | R-19 | 23 | 2.8 | 12.7 |
| Example 20 | R-20 | 25 | 3.0 | 13.0 |
| Example 21 | R-21 | 23 | 2.8 | 12.8 |
| Example 22 | R-22 | 25 | 2.9 | 12.9 |
| Example 23 | R-23 | 22 | 2.8 | 12.7 |
| Example 24 | R-24 | 25 | 2.9 | 13.0 |
| Example 25 | R-25 | 28 | 3.1 | 13.3 |
| Example 26 | R-26 | 28 | 3.0 | 13.2 |
| Example 27 | R-27 | 26 | 2.9 | 13.0 |
| Example 28 | R-28 | 28 | 2.9 | 13.1 |
| Example 29 | R-29 | 25 | 2.8 | 13.0 |
| Example 30 | R-30 | 26 | 2.9 | 13.3 |
| Example 31 | R-31 | 28 | 3.3 | 13.6 |
| Example 32 | R-32 | 31 | 3.5 | 14.0 |
| Example 33 | R-33 | 23 | 2.9 | 13.3 |
| Example 34 | R-34 | 26 | 2.8 | 13.4 |
| Example 35 | R-35 | 23 | 2.8 | 13.8 |
| Example 36 | R-36 | 24 | 2.7 | 13.5 |
| Example 37 | R-37 | 23 | 2.9 | 13.2 |
| Example 38 | R-38 | 26 | 3.2 | 13.0 |
| Example 39 | R-39 | 25 | 2.9 | 13.6 |
| Example 40 | R-40 | 26 | 3.3 | 13.1 |
| Comparative | CR-1 | 33 | 3.6 | 14.7 |
| Example 1 | | | | |
| Comparative | CR-2 | 32 | 3.5 | 14.9 |
| Example 2 | | | | |

As is clear from Table 3, all the radiation-sensitive compositions of Examples 1 to 40 were found to exhibit more excellent sensitivity, LWR performance, and process margin, as compared with the radiation-sensitive compositions of Comparative Examples 1 and 2.

On the basis of the aforementioned test results, the radiation-sensitive composition of the present disclosure which contains a polymer including a structural unit represented by the aforementioned formula (1) exhibits suitable sensitivity to exposure light, a wide process margin, and excellent LWR performance. Thus, the radiation-sensitive composition of the present disclosure, and the resist pattern formation method of the present disclosure employing the composition are suitable for processing of semiconductor devices and the like, which conceivably require further process shrinkage.

1: A radiation-sensitive composition comprising a polymer comprising a structural unit represented by formula (1): 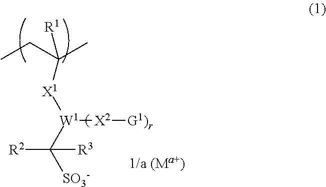
wherein R¹ represents a hydrogen atom, a fluorine atom, a methyl group, or a trifluoromethyl group; X¹ represents a single bond, *¹—COO—, *¹—CONH—, or a divalent aromatic cyclic group; *¹ represents a chemical bond with the carbon atom to which R^{1T} bonds; W¹ represents a (r+2)-valent organic group; X² represents *³—COO— or —O—; *³ represents a chemical bond with W¹; G¹ represents an acid-releasable group; r is 1 or 2; when r is 2, two G¹s in the formula are identical to or different from each other; each of R² and R³ independently represents a fluorine atom or a fluoroalkyl group; M^{a+} represents an a-valent cation; and a is 1 or 2.

2: The radiation-sensitive composition according to claim 1, further comprising a compound (z) which generates an acid having an acidity lower than an acidity of the acid generated by the polymer upon exposure to light.

3: The radiation-sensitive composition according to claim 2, wherein the compound (z) is an onium salt comprising: a cation; and an organic anion comprising an iodine atom.

4: The radiation-sensitive composition according to claim 3, wherein the organic anion comprises an aromatic ring and the iodine atom in the organic anion is bound to the aromatic ring.

5: The radiation-sensitive composition according to claim 2, wherein the compound (z) is an onium salt comprising: an organic anion; and a cation comprising a fluorine atom.

6: The radiation-sensitive composition according to claim 5, wherein
the cation in the compound (z) comprises: a sulfonium cation or an iodonium cation; an aromatic ring R^{A} bound to the sulfonium cation or the iodonium cation; and at least one group selected from the group consisting of a fluoroalkyl group and a fluoro group, which is other than a fluoro group in the fluoroalkyl group, the at least one group being bound to the aromatic ring R^{A}.

7: The radiation-sensitive composition according to claim 1, wherein W¹ in the formula (1) comprises at least one ring selected from the group consisting of an aromatic hydrocarbon ring, an aliphatic hydrocarbon ring, or an aliphatic heterocycle, and the group represented by —X²-G¹ is bound to the at least one ring.

8: The radiation-sensitive composition according to claim 1, wherein W¹ in the formula (1) comprises a chain structure, and the group represented by —X²-G¹ is bound to the chain structure.

9: The radiation-sensitive composition according to claim 1, wherein the polymer comprises a structural unit which comprises: an aromatic ring; and a hydroxy group bound to the aromatic ring.

10: The radiation-sensitive composition according to claim 1, wherein the polymer comprises a structural unit comprising: an acid-releasable group; and no onium salt structure.

11: A resist pattern formation method, comprising
forming a resist film on a substrate by applying a radiation-sensitive composition according to claim 1,
exposing the resist film to light, and
developing the light-exposed resist film.

12: A polymer comprising a structural unit represented by formula (1): 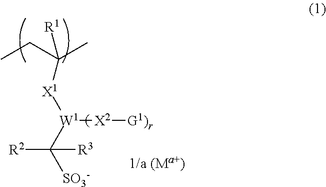
wherein R¹ represents a hydrogen atom, a fluorine atom, a methyl group, or a trifluoromethyl group; X¹ represents a single bond, *¹—COO—, *¹—CONH—, or a divalent aromatic cyclic group; *¹ represents a chemical bond with the carbon atom to which R¹ bonds; W¹ represents a (r+2)-valent organic group; X² represents *³—COO— or —O—; *³ represents a chemical bond with W¹; G¹ represents an acid-releasable group; r is 1 or 2; when r is 2, two G¹s in the formula are identical to or different from each other; each of R² and R³ independently represents a fluorine atom or a fluoroalkyl group; M^{a+} represents an a-valent cation; and a is 1 or 2.

13: A compound represented by formula (2): 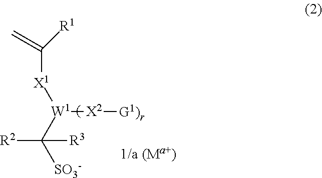
wherein R¹ represents a hydrogen atom, a fluorine atom, a methyl group, or a trifluoromethyl group; X¹ represents a single bond, *¹—COO—, *¹—CONH—, or a divalent aromatic cyclic group; *¹ represents a chemical bond with the carbon atom to which R¹ bonds; W¹ represents a (r+2)-valent organic group; X² represents *³—COO— or —O—; *³ represents a chemical bond with W¹; G¹ represents an acid-releasable group; r is 1 or 2; when r is 2, two G¹s in the formula are identical to or different from each other; each of R² and R³ independently represents a fluorine atom or a fluoroalkyl group; M^{a+} represents an a-valent cation; and a is 1 or 2.
